(19) United States
(12) Patent Application Publication
(10) Pub. No.: US 2014/0323476 A1
(43) Pub. Date: Oct. 30, 2014

(54) PEPTIDE DEFORMYLASE INHIBITORS

(71) Applicant: GlaxoSmithKline Intellectual Preperty (No. 2) Limited, Brentford (GB)

(72) Inventors: Kelly Marshall Aubart, King of Prussia, PA (US); Jason Michael Gillian, King of Prussia, PA (US); Donghui Qin, King of Prussia, PA (US); Robert Rahn McKeown, King of Prussia, PA (US); Glenn R. Williams, King of Prussia, PA (US)

(21) Appl. No.: 14/362,209

(22) PCT Filed: Nov. 30, 2012
(86) PCT No.: PCT/US12/67235

§ 371 Date: Jun. 2, 2014

Related U.S. Application Data
(60) Provisional application No. 61/566,263, filed on Dec. 2, 2011.
(60) Provisional application No. 61/566,398, filed on Dec. 2, 2011.

(51) Int. Cl. C07D 498/04; C07C 309/19; C07C 309/66

(52) U.S. Cl. CPC C07D 498/04; C07C 309/66; C07C 309/19
USPC 514/230.5; 544/105

(57) ABSTRACT

The present invention relates to {2-(alkyl)-3-[2-(5-fluoro-4-pyrimidinyl)hydrazino]-3-oxopropyl}hydroxyformamide compounds of Formula (I):

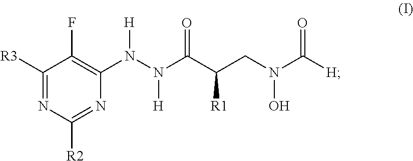

or pharmaceutically acceptable salts thereof, corresponding pharmaceutical compositions, processes for making and use of such compounds in the inhibition of bacterial peptide deformylase (PDF) activity and in treatment methods for bacterial infections.

### FIELD OF THE INVENTION

In general, the present invention relates to {2-(alkyl)-3-[2-(5-fluoro-4-pyrimidinyl)hydrazino]-3-oxopropyl}hydroxyformamide compounds of Formula (I) or pharmaceutically acceptable salts thereof, corresponding pharmaceutical compositions, processes for making and use of such compounds in the inhibition of bacterial peptide deformylase (PDF) activity and in treatment methods for bacterial infections.

### BACKGROUND OF THE INVENTION

Bacterial protein synthesis starts with N-formyl-methionyl-tRNA (f-Met-tRNAᵢ) and, as a consequence, all newly synthesized polypeptides contain an N-formyl-methionine terminus (f-Met-pp) (Scheme I). Peptide deformylase (PDF) is a metalloenzyme that removes the N-formyl group of the polypeptides as they emerge from the ribosome during the elongation process [Adams, J. M. (1968) J. Mol. Biol. 33, 571-589; Livingston, D. M. and Leder, P. (1969) Biochemistry 8, 435-443; Ball, L. A. and Kaesberg, P. (1973) J. Mol. Biol. 79, 531-537]. Depending on the nature of their second amino acid, polypeptides are further processed by methionine amino peptidase (MAP) to yield the mature protein. Deformylation plays an indispensable role in protein maturation as MAP, an essential enzyme for bacterial growth, cannot hydrolyze N-blocked peptides.

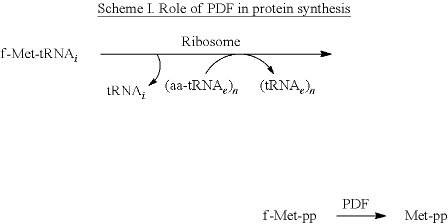

PDF is ubiquitous in bacteria, with at least one pdf gene present in all bacterial genomes sequenced to date.

PDF does not play a role in eukaryotic cytoplasmic protein synthesis which does not involve N-formylation, but nuclear-encoded PDF proteins, containing a chloroplast/mitochondria localization signal, have been identified in parasites, plants and mammals, including humans. PDF is essential in plant and parasite organelles since their genomes encode for a number of proteins which require deformylation for activity, but there is evidence to suggest that this is not the case in animals. In fact, characterization of human mitochondrial PDF has shown that it is much less active than its bacterial counterpart. Furthermore, PDF inhibitors which are active against the human PDF enzyme in vitro have no effect on the growth of normal human cell lines [Nguyen, K. T., Hu, X., Colton, C., Chakrabarti, R., Zhu, M. X. and Pei, D. (2003) Biochemistry 42, 9952-9958].

Thus, PDF inhibitors represent a promising new class of antibacterial agents with a novel mode of action covering a broad-spectrum of pathogens.

PDF inhibitors have been described in the art. Patent applications have been filed on hydrazine-3-oxopropyl hydroxyformamide derivatives of the following formula, see WO 03/101442 and WO2006/055663

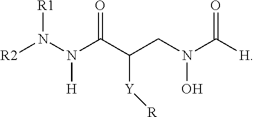

Also WO09/061,879 (i.e., which corresponds to U.S. Pat. No. 7,893,056 to Qin et al., Issued: Feb. 22, 2011) discloses [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide, which is the compound structure as shown below:

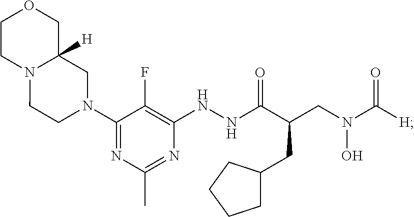

or
a pharmaceutically acceptable salt thereof.

Thus, attempts have been made to prepare compounds that inhibit PDF activity and a number of such compounds have been disclosed in the art. However, there remains a continuing need for inhibitors of PDF which can be used in the treatment of bacterial infections.

In light of the foregoing, a pharmaceutically acceptable salt, and/or novel crystalline form of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide with greater aqueous solubility, chemical stability, etc. would offer many potential benefits for provision of medicinal products, especially for inhibition of bacterial peptide deformylase (PDF) activity and in treatment methods for bacterial infections.

Surprisingly, it has now been shown that novel pharmaceutically acceptable salts of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide can be isolated as pure, crystalline solids, which exhibit much higher aqueous solubility than the corresponding free base. Such novel crystalline forms also improve aqueous solubility and stability in solution of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide or pharmaceutically acceptable salts thereof, in respective salt forms or in pharmaceutical compositions or formulations.

There also exists a need to develop methods for inhibition of bacterial peptide deformylase (PDF) activity and for bacterial infections, which comprises administration of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide or pharmaceutically acceptable salts thereof or corresponding pharmaceutical compositions or formulations.

The present invention is directed to overcoming these and other problems encountered in the art.

### SUMMARY OF THE INVENTION

In general, the present invention relates to pharmaceutically acceptable salts of {2-(alkyl)-3-[2-(5-fluoro-4-pyrimidinyl)hydrazino]-3-oxopropyl}hydroxyformamide compounds of Formula (I), corresponding pharmaceutical compositions, processes for making and use of such compounds in the inhibition of bacterial peptide deformylase (PDF) activity and in treatment methods for bacterial infections.

In particular, the present invention relates to novel salts of compounds of Formula (I), which may include, but are not limited to
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate Forms 1 and 2;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate); and
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate, and
   corresponding pharmaceutical compositions or formulations.

The present invention also relates to processes for making pharmaceutically acceptable salts of compounds of Formula (I).

The present invention also relates to methods for treating bacterial infections, which comprises administering to a subject in need thereof an effective amount of a salt of a compound of Formula (I) or a corresponding pharmaceutical composition.

### BRIEF DESCRIPTION OF THE DRAWINGS

FIG. 1 relates to a ¹H NMR Spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 in DMSO-d₆.

FIG. 2 relates to a ¹³C NMR Spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 in DMSO-d₆.

FIG. 3 relates to an X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1.

FIG. 4 relates to an ATR-IR Spectrum of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1.

FIG. 5 relates to a Differential Scanning calorimetry of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1.

FIG. 6 relates to a Thermogravimetric Analysis of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1.

FIG. 7 relates to an X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate.

FIG. 8 relates to a Differential Scanning calorimetry (DSC) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate.

FIG. 9 relates to a Thermo-Gravimetric Analysis (TGA) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate.

FIG. 10 relates to an X-Ray Powder Diffraction Pattern of 1:1 [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

FIG. 11: Differential Scanning calorimetry of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide camphorsulfonate.

FIG. 12: ¹³C Solid State NMR (¹³C SSNMR) spectrum of crystalline anhydrate of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 1.

FIG. 13: ¹⁹F Solid State NMR (¹⁹F SSNMR) spectrum of crystalline anhydrate of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 1.

FIG. 14: ATR-IR Spectrum of [(2R)-2-(cyclopentylmethyl)-3-(2-{5-fluoro-6-[(9aS)-hexahydro pyrazino[2,1-c][1,4]oxazin-8(1h)-yl]-2-methyl-4-pyrimidinyl}hydrazino)-3-oxopropyl]hydroxyl formamide methanesulphonate polymorphic Form 2.

FIG. 15: X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2.

FIG. 16: ¹H Nuclear Magnetic Resonance Solution State Spectrum (¹H NMR) of [(2R)-2-(Cyclopentyl methyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2 in DMSO-d₆ at 25° C.

FIG. 17: ¹³C Solid State NMR (SSNMR) spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic crystalline anhydrate Form 2.

FIG. 18: ¹⁹F Solid State NMR (SSNMR) spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic crystalline anhydrate Form 2.

FIG. 19: ¹H Nuclear Magnetic Resonance Solution State Spectrum (¹H NMR) for [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide camphorsulfonate at 25° C. in DMSO-d₆

FIG. 20: X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2.

### DETAILED DESCRIPTION OF THE INVENTION

In general, the present invention relates to pharmaceutically acceptable salts of {2-(alkyl)-3-[2-(5-fluoro-4-pyrimidinyl)hydrazino]-3-oxopropyl}hydroxyformamide compounds of Formula (I), corresponding pharmaceutical compositions, processes for making and use of such compounds in the inhibition of bacterial peptide deformylase (PDF) activity and in treatment methods for bacterial infections.

In particular, the present invention relates to novel salts of compounds of Formulas (I), which may include, but are not limited to
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate Forms 1 and 2;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate); and
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate; and
   corresponding pharmaceutical compositions or formulations.

The present invention also relates to processes for making salts of compounds of Formula (I).

The present invention also relates to methods for treating bacterial infections, which comprises administering to a subject in need thereof an effective amount of a salt of a compound of Formula (I) or a corresponding pharmaceutical composition.

### Compounds

In general, the present invention relates to {2-(alkyl)-3-[2-(5-fluoro-4-pyrimidinyl)hydrazino]-3-oxopropyl}hydroxyformamide compounds of Formula (I) or pharmaceutically acceptable salts thereof.

WO09/061,879 (corresponds to U.S. Pat. No. 7,893,056 to Qin et al., Issued: Feb. 22, 2011) discloses [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide, which is the compound structure as shown below:

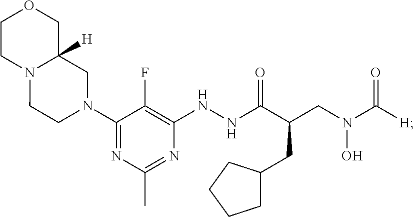

or
a pharmaceutically acceptable salt thereof.

In light of the above, in one aspect, the present invention relates to a compound of Formula (I):

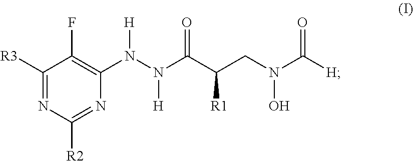

where:

R1 is selected from the group consisting of C2-C7 alkyl and —(CH₂)ₙ—C3-C6 cycloalkyl;

R2 is selected from the group consisting of C1-C3 alkyl; cyclopropyl; C1-C3 alkoxy; C1-C3 haloalkyl; C1-C3 sulfanyl; 5-membered heteroaryl; 5-membered heterocycloalkyl; halo; hydroxymethyl; and —NRaRb;

R3 is selected from the group consisting of —NR4R5; halo; phenyl, optionally substituted by one to three R6 groups; and heteroaryl, optionally substituted by one to three R6 groups;

R4 is selected from the group consisting of H; C1-C6 alkyl, optionally substituted with one or two R7 groups; C1-C6 alkoxy; C3-C6 cycloalkyl, optionally substituted with one to three R6 groups; heterocycloalkyl, optionally substituted by one to three R6 groups; heteroaryl, optionally substituted by one to three R6 groups; and phenyl, optionally substituted by one to three R6 groups;

R5 is selected from H; C1-C6 alkyl, optionally substituted with one or two R7 groups; C1-C6 alkoxy; C3-C6 cycloalkyl, optionally substituted with one to three R6 groups; heterocycloalkyl, optionally substituted by one to three R6 groups; heteroaryl, optionally substituted by one to three R6 groups; and phenyl, optionally substituted by one to three R6 groups; or

R4 and R5 are joined together with the N-atom to which they are attached, forming a heterocycloalkyl group optionally substituted with one to three R6 groups;

where:
each R6 is independently selected from the group consisting of C1-C6 alkyl, optionally substituted with one to three R7 groups; hydroxy; C1-C3 alkoxy; —C(O)NRaRb; —C(O)Rc; —C(O)ORc; heterocycloalkyl; C3-C6 cycloalkyl optionally substituted with one —NRaRb or pyrrolidinyl; oxo; cyano; —NRaRb; phenyl; heteroaryl; and halo;
   each R7 is independently selected from the group consisting of hydroxy; C1-C3 alkoxy; halo; phenyl; cyano; —NRaRb; —C(O)NRaRb; —C(O)Rc; C3-C6 cycloalkyl, optionally substituted with one hydroxy, heterocycloalkyl or —NRaRb group; heterocycloalkyl; and heteroaryl optionally substituted with one methyl, —NRaRb or hydroxy;
each Ra as defined above is independently selected from the group consisting of H and C1-C3 alkyl optionally substituted with one hydroxy, methoxy, or dimethylamine;
each Rb as defined above is independently selected from the group consisting of H and C1-C3 alkyl;
each Rc as defined above is independently selected from the group consisting of C1-C3 alkyl optionally substituted with one methoxy group; phenyl; heterocycloalkyl; and heteroaryl; and
   n is an integer from 0 to 2; or
   a pharmaceutically acceptable salt thereof.

In one aspect of the present invention R1 is —(CH₂)ₙ—C3-C6 cycloalkyl. Suitably, R1 is —(CH₂)ₙ—C3-C6 cycloalkyl wherein n is 1. Suitably R1 is —CH₂-cyclopentyl.

In another embodiment of the present invention R2 is C1-C3 alkyl; C1-C3 alkoxy; C1-C3 haloalkyl; C1-C3 sulfanyl; or halo. Suitably R2 is methyl; ethyl; thiomethyl; thioethyl; fluoromethyl; difluoromethyl; 1-fluoromethyl; chloro; cyclopropyl; or methoxy. Suitably R2 is methyl; ethyl; thiomethyl; or chloro.

In another aspect of the present invention R3 is —NR4R5; C1-C6 alkoxy; or heteroaryl, optionally substituted by one to three R6 groups. Suitably R3 is —NR4R5 wherein R4 is C1-C6 alkyl, optionally substituted with one or two R7 groups; or C3-C6 cycloalkyl, optionally substituted by one to three R6 groups; and R5 is H, C1-C6 alkyl, or C1-C6 alkoxy.

In another aspect of the present invention R4 is cyclopropyl; cyclobutyl; cyclopentyl; tetrahydro-2H-pyranyl; 2-oxohexahydro-1H-azepinyl; 2-oxo-2,3,4,7-tetrahydro-1H-azepinyl; 5-fluoro-pyridinyl; or C1-C6 alkyl optionally substituted with one of the following R7 groups selected from the group consisting of hydroxyl; methoxy; cyano; —C(O)NRaRb; —C(O)Rc; morpholinyl; pyridinyl; 1,3-thiazolyl; 2-amino-1,3-thiazoyl; thienyl; furanyl; phenyl; and 1-hydroxy-1H-imidazolyl; and R5 is H; C1-C3 alkyl; cyclopropyl; or piperazinyl optionally substituted with one R6 group.

In another aspect R4 is methyl; ethyl optionally substituted with one substituent selected from the group consisting of: hydroxyl, methoxy and —NRaRb; propyl; isopropyl; cyclopropyl; cyclobutyl; and cyclopentyl.

In another aspect of the present invention R5 is selected from the group consisting of H; C1-C6 alkyl; C1-C6 alkoxy; and C3-C6 cycloalkyl. Suitabley R5 is H; methyl; or methoxy. Suitably R5 is H; C1-C3 alkyl; cyclopropyl; or piperazinyl optionally substituted with one R6 group.

In another aspect of the present invention R3 is —NR4R5 wherein R4 and R5 are joined together with the N-atom to which they are attached to form a heterocycloalkyl group optionally substituted with one to three R6 groups, where heterocycloalkyl groups may include, but are not limited to monocyclic ring systems or are fused, spiro, or bridged bicyclic ring systems.

Suitably R3 is —NR4R5 wherein R4 and R5 are joined together with the N-atom to which they are attached forming azetidinyl; pyrrolidinyl; piperazinyl; morpholinyl; 2,5-dihydro-1H-pyrrolyl; hexahydropyrazino[2,1-c][1,4]oxazin-(1H)-yl; isoxazolidinyl; hexahydropyrrolo[1,2-a]pyrazin-(1H)-yl; or 2,5-diazabicyclo[2.2.1]heptyl each of which may be optionally substituted with one to three R6 groups.

In another aspect of the present invention R3 is —NR4R5 wherein R4 and R5 are joined together with the N-atom to which they are attached forming 1-piperidinyl; 4-thiomorpholinyl; 1-pyrazolidinyl; tetrahydro-5H-[1,3]dioxolo[4,5-c]pyrrolyl; tetrahydro-1H-furo[3,4-c]pyrrol-(3H)-yl; hexahydropyrrolo[3,4-c]pyrrol-(1H)-yl; hexahydropyrrolo[1,2-a]pyrazin-(1H)-yl; hexahydropyrazino[2,1-c][1,4]oxazin-(1H)-yl; hexahydrofuro[3,4-b]pyrazin-(2H)-yl; octahydro-2H-pyrido[1,2-a]pyrazinyl; octahydropyrazino[1,2-a]azepin-(1H)-yl; octahydropyrazino[2,1-c][1,4]oxazinyl; octahydro-1H-cyclopenta[b]pyrazinyl; octahydro-1(2H)-quinoxalinyl; octahydro-6H-pyrrolo[3,4-b]pyridinyl; 3-azabicyclo[3.1.0]hexyl; 2,5-diazabicyclo[2.2.1]heptyl; 4,7-diazaspiro[2.5]octyl; 5-azaspiro[2.4]heptyl; or 10-oxa-4-azatricyclo[5.2.1.02,6]decyl.

Suitably R3 is —NR4R5 wherein R4 and R5 are joined together with the N-atom to which they are attached forming azetidinyl optionally substituted with one or two R6 groups each independently selected from the group consisting of methyl; ethyl; fluoro; methoxy; hydroxyl; hydroxymethyl; cyclopropyl; dimethylamino; ethylmethylamino; —CH₂-dimethylamino; morpholinyl; pyrrolidinyl; —CH₂-pyrrolidinyl; and pyridinyl.

Suitably R3 is —NR4R5 wherein R4 and R5 are joined together with the N-atom to which they are attached forming pyrrolidinyl optionally substituted with one to three R6 groups each independently selected from the group consisting of methyl; methoxy; —CH₂-methoxy; hydroxyl; hydroxymethyl; hydroxyethyl; dimethylamino; ethylmethylamino; —CH₂-dimethylamino; —CH₂-pyrrolidinyl; —CH₂-morpholinyl; pyridinyl; 2-(dimethylamino)-1,1-dimethylethyl; fluoromethyl; —CH₂-2-hydroxyethylmethylamino; —CH₂-2-methoxyethylamino; cyano; —C(O)N(CH₃)₂; 1-(dimethyamino)cyclopropyl; —CH₂-ethylemethylamino; —CH₂-diethylamino; —C(O)N(CH₂CH₃)₂; —CH₂-piperidinyl; —CH₂-isopropylmethylamino; —CH₂-propylmethylamino; —NHCOOCH₃; —CH₂-3H-[1,2,3]triazolo[4,5-d]pyrimidin-3-yl; and (cis)-10-oxa-4-azatricyclo[5.2.1.0^{2,6}]dec-4-yl.

Suitably R3 is —NR4R5 wherein R4 and R5 are joined together with the N-atom to which they are attached forming piperazinyl optionally substituted with one to three R6 groups each independently selected from the group consisting of methyl; ethyl; isopropyl; hydroxymethyl; hydroxyethyl; —CH₂—O—CH₃; and —COOCH₃.

Suitably R3 is —NR4R5 wherein R4 and R5 are joined together with the N-atom to which they are attached forming (9aS)-octahydropyrazino[2,1-c][1,4]oxazinyl.

In another aspect of the present invention R6 is C1-C3 alkyl, optionally substituted with one to three R7 groups; hydroxy; C1-C3 alkoxy; —C(O)NRaRb; or —NRaRb. Suitably R6 is methyl; ethyl; isopropyl; methoxy; hydroxyl; diethylamino; or N,N-dimethylacetamido.

In another aspect of the present invention R6 is heteroaryl. Suitably R6 is a 6-membered heteroaryl. Suitably R6 is pyridinyl.

In another aspect of the present invention R7 is C1-C3 alkoxy; hydroxyl; or —NRaRb. Suitably R7 is methoxy.

In another aspect of the present invention R7 is heterocycloalkyl. Suitably R7 is a 6-membered heterocycloalkyl. Suitably R7 is morpholinyl.

In another aspect of the present invention R7 is heteroaryl. Suitably R7 is pyridinyl; 1,3-thiazolyl; thienyl; furanyl; imidazolyl; 1H-benzamidazolyl; 3H-[1,2,3]triazolo[4,5-d]pyrimidin-3-yl; or 3H-[1,2,3]triazolo[4,5-b]pyridin-3-yl.

In another aspect of the present invention Ra and Rb are both methyl.

In another aspect of the present invention Rc is heterocycloalkyl. Suitabley Rc is pyrrolidinyl.

In another aspect of the present invention is a compound according to Formula (I):

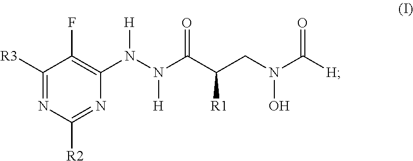

where:

R1 is selected from the group consisting of C2-C7 alkyl and —(CH₂)ₙ—C3-C6 cycloalkyl;

R2 is selected from the group consisting of C1-C3 alkyl; cyclopropyl; C1-C3 alkoxy; C1-C3 haloalkyl; C1-C3 sulfanyl; 5-membered heteroaryl; 5-membered heterocycloalkyl; halo; hydroxymethyl; and —NRaRb;

R3 is selected from the group consisting of —NR4R5; halo; phenyl, optionally substituted by one to three R6 groups; and heteroaryl, optionally substituted by one to three R6 groups;

R4 is selected from the group consisting of H; C1-C6 alkyl, optionally substituted with one or two R7 groups; C1-C6 alkoxy; C3-C6 cycloalkyl, optionally substituted with one to three R6 groups; heterocycloalkyl, optionally substituted by one to three R6 groups; heteroaryl, optionally substituted by one to three R6 groups; and phenyl, optionally substituted by one to three R6 groups;

R5 is selected from the group consisting of H; C1-C6 alkyl, optionally substituted with one or two R7 groups; C1-C6 alkoxy; C3-C6 cycloalkyl, optionally substituted with one to three R6 groups; heterocycloalkyl, optionally substituted by one to three R6 groups; heteroaryl, optionally substituted by one to three R6 groups; and phenyl, optionally substituted by one to three R6 groups; or

R4 and R5 are joined together with the N-atom to which they are attached, forming a heterocycloalkyl group optionally substituted with one to three R6 groups;

where:
each R6 is independently selected from the group consisting of C1-C6 alkyl, optionally substituted with one to three R7 groups; hydroxy; C1-C3 alkoxy; —C(O)NRaRb; —C(O)Rc; heterocycloalkyl; C3-C6 cycloalkyl; oxo; cyano; —NRaRb; phenyl; heteroaryl; and halo;
   each R7 is independently selected from the group consisting of hydroxy; C1-C3 alkoxy; halo; phenyl; cyano; —NRaRb; —C(O)NRaRb; —C(O)Rc; C3-C6 cycloalkyl, optionally substituted with one hydroxy, heterocycloalkyl or —NRaRb group; heterocycloalkyl; and heteroaryl;

each Ra as defined above is independently selected from the group consisting of H and C1-C3 alkyl;

each Rb as defined above is independently selected from the group consisting of H and C1-C3 alkyl;

each Rc as defined above is independently selected from the group consisting of C1-C3 alkyl; phenyl; heterocycloalkyl; and heteroaryl; and

n is an integer from 0 to 2; or
a pharmaceutically acceptable salt thereof.

In another aspect of the present invention is a compound according to Formula (I):

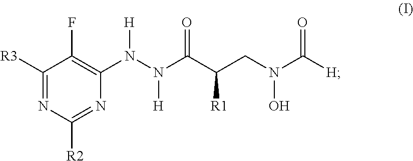

where:

R1 is —CH₂-cyclopentyl;

R2 is selected from the group consisting of methyl; ethyl; thiomethyl; thioethyl; fluoromethyl; difluoromethyl; 1-fluoromethyl; chloro; cyclopropyl; or methoxy;

R3 is —NR4R5;

R4 is selected from the group consisting of H; C1-C3 alkyl; cyclopropyl; and piperazinyl optionally substituted with one R6 group;

R5 is selected from the group consisting of H; C1-C6 alkyl, optionally substituted with one or two R7 groups; C1-C6 alkoxy; C3-C6 cycloalkyl, optionally substituted with one to three R6 groups; heterocycloalkyl, optionally substituted by one to three R6 groups; heteroaryl, optionally substituted by one to three R6 groups; and phenyl, optionally substituted by one to three R6 groups; or

R4 and R5 are joined together with the N-atom to which they are attached, forming a heterocycloalkyl group optionally substituted with one to three R6 groups;

where:
each R6 is independently selected from the group consisting of C1-C6 alkyl, optionally substituted with one to three R7 groups; hydroxy; C1-C3 alkoxy; —C(O)NRaRb; —C(O)Rc; C(O)ORc; heterocycloalkyl; C3-C6 cycloalkyl optionally substituted with one —NRaRb or pyrrolidinyl; oxo; cyano; —NRaRb; phenyl; heteroaryl; and halo;
   each R7 is independently selected from the group consisting of hydroxy; C1-C3 alkoxy; halo; phenyl; cyano; —NRaRb; —C(O)NRaRb; —C(O)Rc; C3-C6 cycloalkyl, optionally substituted with one hydroxy, heterocycloalkyl or —NRaRb group; heterocycloalkyl; and heteroaryl optionally substituted with one methyl, —NRaRb or hydroxy;

each Ra is each independently selected from H and C1-C3 alkyl optionally substituted with one hydroxy, methoxy group or dimethylamine;

each Rb is independently selected from H and C1-C3 alkyl; and

each Rc is independently selected from C1-C3 alkyl optionally substituted with one methoxy group; phenyl; heterocycloalkyl; and heteroaryl; or

a pharmaceutically acceptable salt thereof.

In one aspect, the present invention relates to [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide or a pharmaceutically acceptable salt thereof.

In one aspect, the present invention relates to [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate.

In one aspect, the present invention relates to [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate).

In one aspect, the present invention relates to [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

### TERMS AND DEFINITIONS

“Alkyl” refers to a monovalent saturated hydrocarbon chain having the specified number of member carbon atoms. For example, C1-C7 alkyl refers to an alkyl group having from 1 to 7 member carbon atoms. Alkyl groups may be optionally substituted with one or more substituents as defined herein. Alkyl groups may be straight or branched. Representative branched alkyl groups have one, two, or three branches. Alkyl includes methyl, ethyl, propyl (n-propyl and isopropyl), butyl (n-butyl, isobutyl, and t-butyl), pentyl (n-pentyl, isopentyl, and neopentyl), and hexyl.

“Alkenyl” refers to an unsaturated hydrocarbon chain having the specified number of member carbon atoms and having one or more carbon-carbon double bonds within the chain. For example, C2-C6 alkenyl refers to an alkenyl group having from 2 to 6 member carbon atoms. In certain embodiments, alkenyl groups have one carbon-carbon double bond within the chain. In other embodiments, alkenyl groups have more than one carbon-carbon double bond within the chain. Alkenyl groups may be optionally substituted with one or more substituents as defined herein. Alkenyl groups may be straight or branched. Representative branched alkenyl groups have one, two, or three branches. Alkenyl includes ethylenyl, propenyl, butenyl, pentenyl, and hexenyl.

“Alkoxy” refers to an alkyl moiety attached through an oxygen bridge (i.e. a —O—C1-C6 alkyl group wherein C1-C6 is defined herein). Examples of such groups include methoxy, ethoxy, propoxy, butoxy, pentoxy and hexoxy.

“Alkynyl” refers to an unsaturated hydrocarbon chain having the specified number of member carbon atoms and having one or more carbon-carbon triple bonds within the chain. For example, C2-C6 alkynyl refers to an alkynyl group having from 2 to 6 member atoms. In certain embodiments alkynyl groups have one carbon-carbon triple bond within the chain. In other embodiments, alkynyl groups have more than one carbon-carbon triple bond within the chain. For the sake of clarity, unsaturated hydrocarbon chains having one or more carbon-carbon triple bond within the chain and one or more carbon-carbon double bond within the chain are referred to as alkynyl groups. Alkynyl groups may be optionally substituted with one or more substituents as defined herein. Representative branched alkynyl groups have one, two, or three branches. Alkynyl includes ethynyl, propynyl, butynyl, pentynyl, and hexynyl.

“Aryl” refers to an aromatic hydrocarbon ring system. Aryl groups are monocyclic ring systems or bicyclic ring systems. Monocyclic aryl ring refers to phenyl. Bicyclic aryl rings refer to napthyl and to rings wherein phenyl is fused to a cycloalkyl or cycloalkenyl ring having 5, 6, or 7 member carbon atoms. Aryl groups may be optionally substituted with one or more substituents as defined herein.

“Cycloalkyl” refers to a saturated hydrocarbon ring having the specified number of member carbon atoms. Cycloalkyl groups are monocyclic ring systems. For example, C3-C6 cycloalkyl refers to a cycloalkyl group having from 3 to 6 member atoms. Cycloalkyl groups may be optionally substituted with one or more substituents as defined herein. Cycloalkyl includes cyclopropyl, cyclobutyl, cyclopentyl, and cyclohexyl.

“Cycloalkenyl” refers to an unsaturated hydrocarbon ring having the specified number of member carbon atoms and having a carbon-carbon double bond within the ring. For example, C3-C6 cycloalkenyl refers to a cycloalkenyl group having from 3 to 6 member carbon atoms. In certain embodiments, cycloalkenyl groups have one carbon-carbon double bond within the ring. In other embodiments, cycloalkenyl groups have more than one carbon-carbon double bonds within the ring. Cycloalkenyl rings are not aromatic. Cycloalkenyl groups are monocyclic ring systems. Cycloalkenyl groups may be optionally substituted with one or more substituents as defined herein. Cycloalkenyl includes cyclopropenyl, cyclobutenyl, cyclopentenyl, cyclohexenyl, and cyclohexadienyl.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically enriched” refers to products whose enantiomeric excess is greater than zero. For example, enantiomerically enriched refers to products whose enantiomeric excess is greater than 50% ee, greater than 75% ee, or greater than 90% ee.

“Enantiomerically pure” refers to products whose enantiomeric excess is 99% ee or greater.

“Halo” refers to the halogen radicals fluoro, chloro, bromo, and iodo.

“Haloalkyl” refers to an alkyl group wherein at least one hydrogen atom attached to a member atom within the alkyl group is replaced with halo. The number of halo substituents include but are not limited to 1, 2, 3, 4, 5, or 6 substituents. Haloalkyl includes monofluoromethyl, difluoroethyl, and trifluoromethyl.

“Heteroaryl” refers to an aromatic ring containing from 1 to 5, suitably 1 to 4, more suitably 1 or 2 heteroatoms as member atoms in the ring. Heteroaryl groups containing more than one heteroatom may contain different heteroatoms. Heteroaryl groups may be optionally substituted with one or more substituents as defined herein. Heteroaryl groups are monocyclic ring systems, or are fused bicyclic ring systems. Monocyclic heteroaryl rings have from 5 to 6 member atoms. Bicyclic heteroaryl rings have from 8 to 10 member atoms. Bicyclic heteroaryl rings include those rings wherein the primary heteroaryl and the secondary monocyclic cycloalkyl, cycloalkenyl, heterocycloalkyl, aryl or heteroaryl ring are attached, forming a fused bicyclic ring system. Heteroaryl includes, among others, pyrrolyl, pyrazolyl, imidazolyl, oxazolyl, isoxazolyl, oxadiazolyl, thiazolyl, isothiazolyl, thiadiazolyl, furanyl, furazanyl, thienyl, triazolyl, pyridinyl, pyrimidinyl, pyridazinyl, pyrazinyl, triazinyl, tetrazinyl, tetrazolyl, indolyl, isoindolyl, indolizinyl, indazolyl, purinyl, quinolinyl, isoquinolinyl, quinoxalinyl, quinazolinyl, pteridinyl, cinnolinyl, benzimidazolyl, benzopyranyl, benzoxazolyl, benzisoxazolyl, benzofuranyl, isobenzofuranyl, benzothiazolyl, benzisothiazolyl, benzothienyl, furopyridinyl, napthyridinyl, pyrazolopyridyl, pyrazolopyrimidinyl, 3H-[1,2,3]triazolo[4,5-d]pyrimidinyl, and 3H-[1,2,3]triazolo[4,5-b]pyridinyl.

“Heteroatom” refers to a nitrogen, sulfur, or oxygen atom.

“Heterocycloalkyl” refers to a saturated or unsaturated ring containing from 1 to 4 heteroatoms as member atoms in the ring. Heterocycloalkyl rings are not aromatic. Heterocycloalkyl groups containing more than one heteroatom may contain different heteroatoms. Heterocycloalkyl groups may be optionally substituted with one or more substituents as defined herein. Heterocycloalkyl groups are monocyclic ring systems or are fused, spiro, or bridged bicyclic ring systems. Monocyclic heterocycloalkyl rings have from 4 to 7 member atoms. Bicyclic heterocycloalkyl rings have from 7 to 11 member atoms. In certain embodiments, heterocycloalkyl is saturated. In other embodiments, heterocycloalkyl is unsaturated, but not aromatic. Heterocycloalkyl includes, among others, pyrrolidinyl, tetrahydrofuranyl, dihydrofuranyl, pyranyl, tetrahydropyranyl, dihydropyranyl, tetrahydrothienyl, pyrazolidinyl, oxazolidinyl, thiazolidinyl, piperidinyl, homopiperidinyl, piperazinyl, morpholinyl, thiamorpholinyl, 1-pyrazolidinyl, azepinyl, 1,3-dioxanyl, 1,4-dioxanyl, 1,3-oxathiolanyl, 1,3-dithianyl, azetidinyl, isoxazolidinyl, 3-azabicyclo[3.1.0]hexyl, azabicylo[3.2.1]octyl, azabicylo[3.3.1]nonyl, azabicylo[4.3.0]nonyl, 2,5-diazabicyclo[2.2.1]heptanyl, octahydropyrrolo[1,2-a]pyrazinyl, octahydropyrazino[2,1-c][1,4]oxazinyl, oxabicylo[2.2.1]heptyl, hexahydro-1H-azepinyl,2,3,4,7-tetrahydro-1H-azepinyl, tetrahydro-5H-[1,3]dioxolo[4,5-c]pyrrolyl, tetrahydro-1H-furo[3,4-c]pyrrol-(3H)-yl, hexahydropyrrolo[1,2-a]pyrazin-(1H)-yl, octahydropyrazino[1,2-a]azepin-(1H)-yl, hexahydropyrazino[2,1-c][1,4]oxazin-(1H)-yl, hexahydropyrrolo[3,4-c]pyrrol-(1H)-yl, 10-oxa-4-azatricyclo[5.2.1.02,6]decyl, octahydro-1(2H)-quinoxalinyl; octahydro-1H-cyclopenta[b]pyrazinyl, hexahydrofuro[3,4-b]pyrazin-(2H)-yl, octahydro-6H-pyrrolo[3,4-b]pyridinyl, 4,7-diazaspiro[2.5]octyl, and 5-azaspiro[2.4]heptyl.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Optionally substituted” indicates that a group, such as alkyl, alkenyl, alkynyl, aryl, cycloalkyl, cycloalkenyl, heterocycloalkyl, or heteroaryl, may be unsubstituted, or the group may be substituted with one or more substituents as defined herein.

“Pharmaceutically acceptable” refers to those compounds, materials, compositions, and dosage forms which are, within the scope of sound medical judgment, suitable for use in contact with the tissues of human beings or animals without excessive toxicity, irritation, or other problem or complication, commensurate with a reasonable benefit/risk ratio.

“Substituted” in reference to a group indicates that one or more hydrogen atoms attached to a member atom within the group is replaced with a substituent selected from the group of defined substituents. It should be understood that the term “substituted” includes the implicit provision that such substitution be in accordance with the permitted valence of the substituted atom and the substituent, and that the substitution results in a stable compound (i.e. one that does not spontaneously undergo transformation such as by hydrolysis, rearrangement, cyclization, or elimination, and that is sufficiently robust to survive isolation from a reaction mixture). When it is stated that a group may contain one or more substituents, one or more (as appropriate) member atom within the group may be substituted. In addition, a single member atom within the group may be substituted with more than one substituent as long as such substitution is in accordance with the permitted valence of the atom. Suitable substituents are defined herein for each substituted or optionally substituted group.

“Sulfanyl” refers to an alkyl moiety attached through a sulphur bridge (i.e —S—C1-C6 alkyl group wherein C1-C6 alkyl is as defined herein). Examples of sulfanyl groups include thiomethyl and thioethyl.

For the avoidance of doubt, the following terms generally are used interchangeably, synonymously herein or any variation thereof: Polymorphic Form 1 or 2 of a compound salt of the present invention, Compound Salt Polymorphic Form 1 or 2, Compound Salt Form 1 or 2 and the like. For example, the following terms to describe specific compounds of the present invention are used interchangeably, synonymously or any variation thereof:

with regard to:
Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1;
Polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate; or
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Polymorphic Form 1; and the like; and/or
   with regard to:
Form 2 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2;
Polymorphic Form 2 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate; or
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Polymorphic Form 2; and the like.

### Enantiomers, Diastereomers and Polymorphs

The compounds according to Formula (I) or a pharmaceutically acceptable salt thereof, may contain one or more asymmetric centers (also referred to as a chiral center) and may, therefore, exist as individual enantiomers, diastereomers, or other stereoisomeric forms, or as mixtures thereof.

Chiral centers, such as chiral carbon atoms, may also be present in a substituent such as an alkyl group. Where the stereochemistry of a chiral center present in Formula I, or in any chemical structure illustrated herein, is not specified, the structure is intended to encompass all individual stereoisomers and all mixtures thereof.

Thus, compounds according to Formula (I) or pharmaceutically acceptable salts thereof, containing one or more chiral centers may be used as racemic mixtures, diastereomeric mixtures, enantiomerically enriched mixtures, diastereomerically enriched mixtures, or as enantiomerically and diastereomerically pure individual stereoisomers.

Individual stereoisomers of a compound according to Formula (I) or a pharmaceutically acceptable salt thereof which contain one or more asymmetric centers may be resolved by methods known to those skilled in the art. For example, such resolution may be carried out (1) by formation of diastereoisomeric salts, complexes or other derivatives; (2) by selective reaction with a stereoisomer-specific reagent, for example by enzymatic oxidation or reduction; or (3) by gas-liquid or liquid chromatography in a chiral environment, for example, on a chiral support such as silica with a bound chiral ligand or in the presence of a chiral solvent. The skilled artisan will appreciate that where the desired stereoisomer is converted into a diastereomeric salt, complex or derivative, a further step is required to liberate the desired form. Alternatively, specific stereoisomers may be synthesized by asymmetric synthesis using optically active reagents, substrates, catalysts or solvents, or by converting one enantiomer to the other by asymmetric transformation.

When a disclosed compound or its salt is named or depicted by structure, it is to be understood that the compound or salt, including solvates (particularly, hydrates) thereof, may exist in crystalline forms, non-crystalline forms or a mixture thereof. The compound or salt, or solvates (particularly, hydrates) thereof, may also exhibit polymorphism (i.e. the capacity to occur in different crystalline forms). These different crystalline forms are typically known as “polymorphs.”

In light of this, salt forms of the present invention (i.e., which may include different polymorphs, anhydrous forms, solvates, or hydrates thereof) may exhibit characteristic polymorphism. As conventionally understood in the art, polymorphism is defined as an ability of a compound to crystallize as more than one distinct crystalline or “polymorphic” species. A polymorph is defined as a solid crystalline phase of a compound with at least two different arrangements or polymorphic forms of that compound molecule in the solid state.

Polymorphic forms of any given compound, including those of the present invention, are defined by the same chemical formula or composition and are as distinct in chemical structure as crystalline structures of two different chemical compounds. Such compounds may differ in packing, geometrical arrangement of respective crystalline lattices, etc.

It is to be understood that when named or depicted by structure, the disclosed compound, or solvates (particularly, hydrates) thereof, also include all polymorphs thereof. Polymorphs have the same chemical composition but differ in packing, geometrical arrangement, and other descriptive properties of the crystalline solid state.

In light of the foregoing, chemical and/or physical properties or characteristics vary with each distinct polymorphic form, which may include variations in solubility, melting point, density, hardness, crystal shape, optical and electrical properties, vapor pressure, stability, etc.

Solvates and/or hydrates of crystalline salt forms of the present invention also may be formed when solvent molecules are incorporated into the crystalline lattice structure of the compound molecule during the crystallization process. For example, solvate forms of the present invention may incorporate nonaqueous solvents such as methanol and the like as described herein below. Hydrate forms are solvate forms, which incorporate water as a solvent into a crystalline lattice.

Anhydrous with respect to solid state polymorphism refers to a crystalline structure that does not contain a repeating, crystalline solvent in the lattice. However, crystalline materials can be porous and may exhibit reversible surface adsorption of water.

For example:
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 is an anhydrous, crystalline polymorph of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate.

The aforementioned [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Polymorphic Form 1 can adsorb up to 0.4% (wt/wt) of labile water reversibly up to 70% relative humidity, at which point it deliquesces; and
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 is an anhydrous, crystalline polymorph of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate.

The aforementioned [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 can adsorb up to 2% (wt/wt) labile water reversibly up to 95% relative humidity.

In light of that, novel salt compounds of the present invention may exist as crystalline anhydrous forms or crystalline anhydrates, hydrated forms (i.e., hydrate forms are solvate forms, which incorporate water as a solvent into a crystalline lattice) or mixtures thereof.

In one aspect, the compound (Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 may exist as a crystalline anhydrate or crystalline anhydrous form, a hydrate, or a mixture thereof.

In another aspect, the compound (Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 may exist as a crystalline anhydrate or crystalline anhydrous form, a hydrate, or a mixture thereof.

Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. One of ordinary skill in the art will appreciate that different polymorphs may be produced, for example, by changing or adjusting the conditions used in crystallizing/recrystallizing the compound.

The compounds according to Formula (I) or a pharmaceutically acceptable salt thereof may also contain double bonds or other centers of geometric asymmetry. Where the stereochemistry of a center of geometric asymmetry present in Formula I, or in any chemical structure illustrated herein, is not specified, the structure is intended to encompass the trans (E) geometric isomer, the cis (Z) geometric isomer, and all mixtures thereof. Likewise, all tautomeric forms are also included in Formula I whether such tautomers exist in equilibrium or predominately in one form.

The skilled artisan will further appreciate that certain compounds of the invention that exist in crystalline form, including the various solvates thereof, may exhibit polymorphism (i.e. the capacity to occur in different crystalline structures). These different crystalline forms are typically known as “polymorphs.” The invention includes all such polymorphs.

Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties.

According to the instant invention, the various forms (i.e., which may include, but are not limited to polymorphic, salt, solvate, anhydrate, hydrate, crystalline, forms etc.) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (i.e. which include salts and/or solvates thereof) are distinguished from each other using different characterization or identification techniques. Such techniques, include solid state ¹³C Nuclear Magnetic Resonance (NMR), ³¹P Nuclear Magnetic Resonance (NMR), Infrared (IR), Raman, X-ray powder diffraction, etc. and/or other techniques, such as Differential Scanning calorimetry (DSC) (i.e., which measures the amount of energy (heat) absorbed or released by a sample as it is heated, cooled or held at constant temperature). For example, Polymorphs typically exhibit different melting points, IR spectra, and X-ray powder diffraction patterns, which may be used for identification.

The skilled artisan will appreciate that different polymorphs may be produced, for example, by changing or adjusting the reaction conditions or reagents used in making the compound, or by using different isolation or purification procedures. For example, changes in temperature, pressure, or solvent may result in polymorphs. In addition, one polymorph may spontaneously convert to another polymorph under certain conditions.

As is known, the crystalline state of a compound can be described by several crystallographic parameters: unit cell dimensions, space groups, and atomic position of the atoms in the compound relative to the origin of its unit cell. These parameters are experimentally determined by crystal X-ray analysis. It is possible for a compound to form more than one type of crystal. These different crystalline forms are called polymorphs.

Characteristic powder X-ray diffraction pattern peak positions are reported for polymorphs in terms of the angular positions (two theta) with an allowable variability, generally of about 0.1+/−°2-theta or 0.1+/−°3-theta. The entire pattern, or most of the pattern peaks may also shift by about 0.1+/−° due to difference in calibration, setting, and other variations from instrument to instrument and from operator to operator.

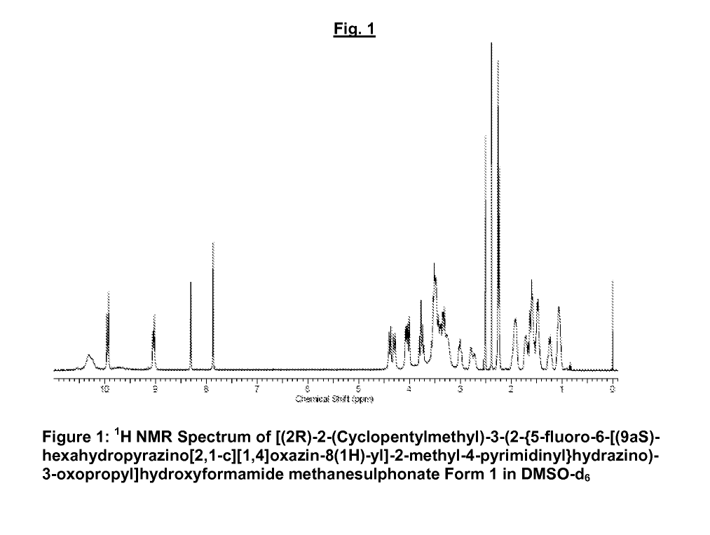

Specifically, the following pharmaceutically acceptable forms of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide or pharmaceutically acceptable salts thereof are substantially shown by the data described in FIGS. 1 to 20:
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate Forms 1 and 2;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate); and
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

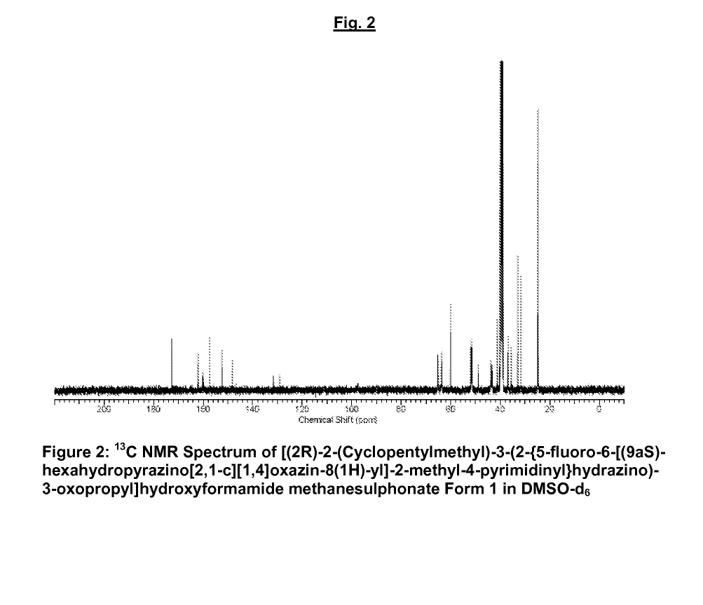

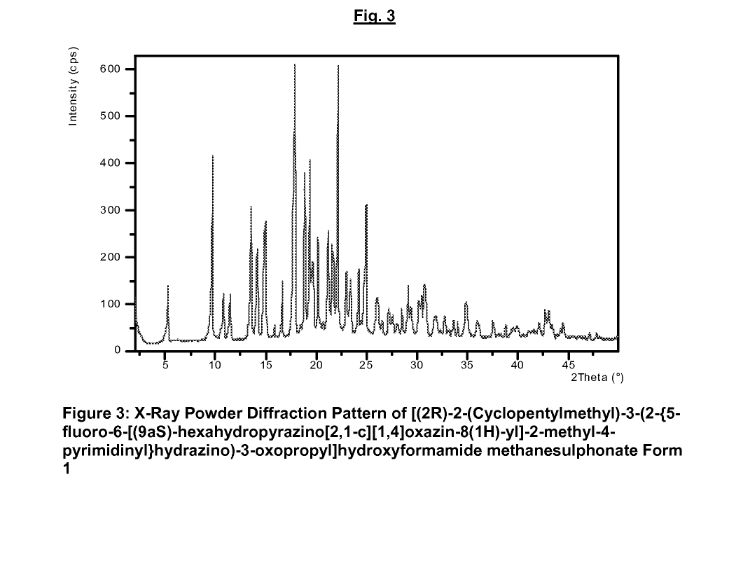

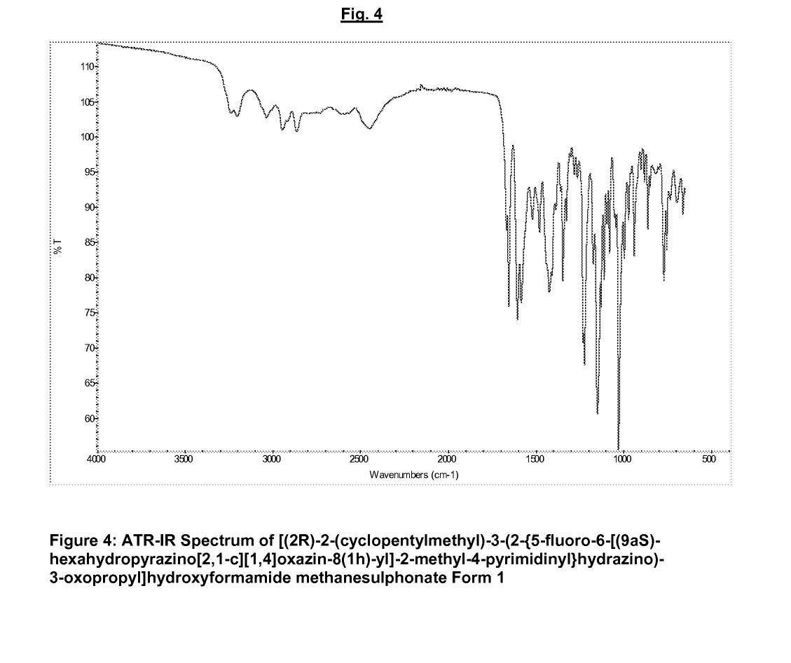

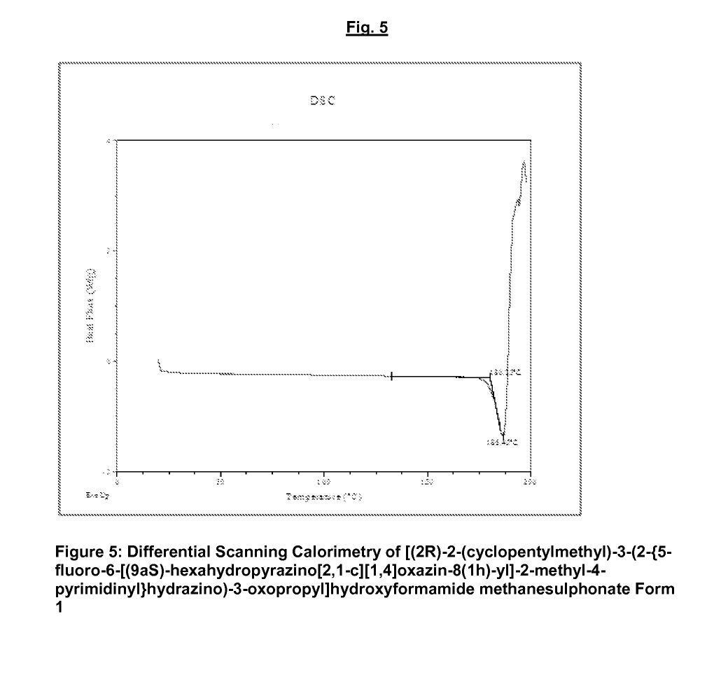

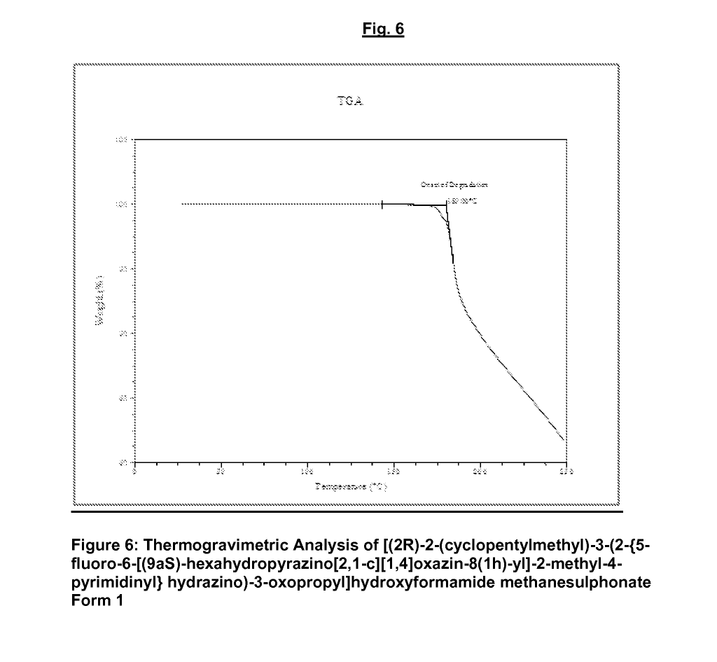

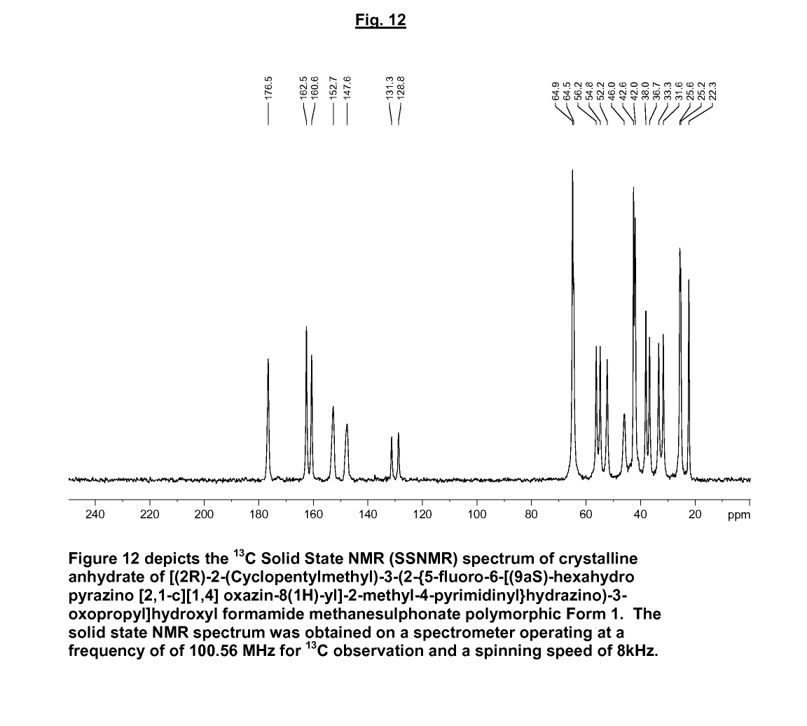

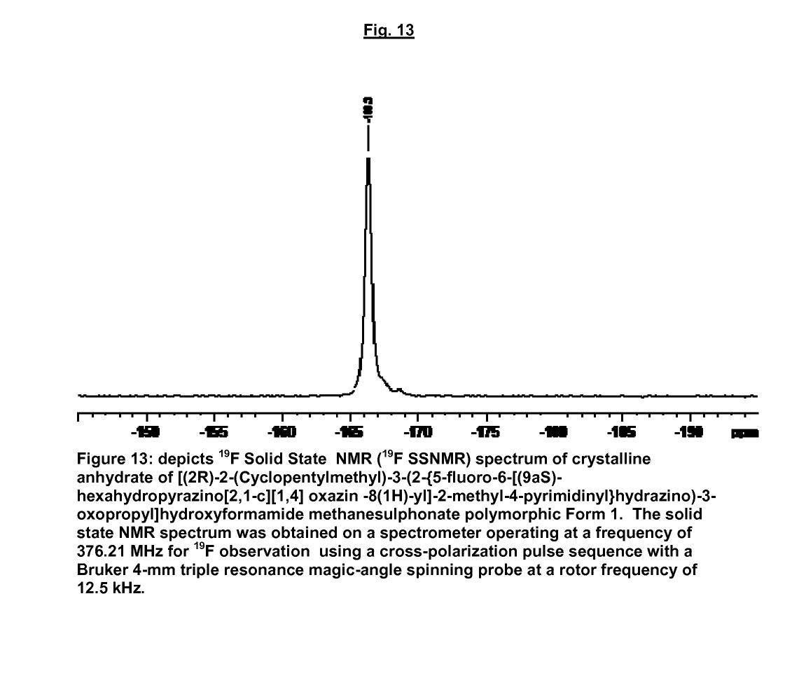

For example, crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 is identified by:
a ¹H NMR and ¹³C NMR Spectrum, respectively, of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 in DMSO-d₆ (see FIGS. 1 and 2, respectively, and Tables 1 and 2, respectively,);
an x-Ray Powder Diffraction Pattern of Form 1 [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 (see Table 3 and FIG. 3);
an X-ray diffraction pattern as shown substantially in FIG. 3, which depicts characteristic peaks from 0° degrees 2-theta (2θ) to 55° degrees 2-theta (2θ) at about 5.3±0.3 (2θ), 9.7±0.3 (2θ), 10.8±0.3 (2θ), 11.4±0.3 (2θ), 13.5±0.3 (2θ), 14.9±0.3 (2θ), 17.8±0.3 (2θ). 18.9±0.3 (2θ). 21.2±0.3 (2θ) and 22.1±0.3 (2θ) (see, Example 6: Form 1);
an ATR-IR Spectrum of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 (see FIG. 4 and Table 4);
a Differential Scanning calorimetry of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 (see FIG. 5);
a Thermogravimetric Analysis of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 (see FIG. 6); and
a ¹³C NMR and ¹⁹F Solid State Nuclear Magnetic Resonance Spectra of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 (see FIGS. 12 and 13).

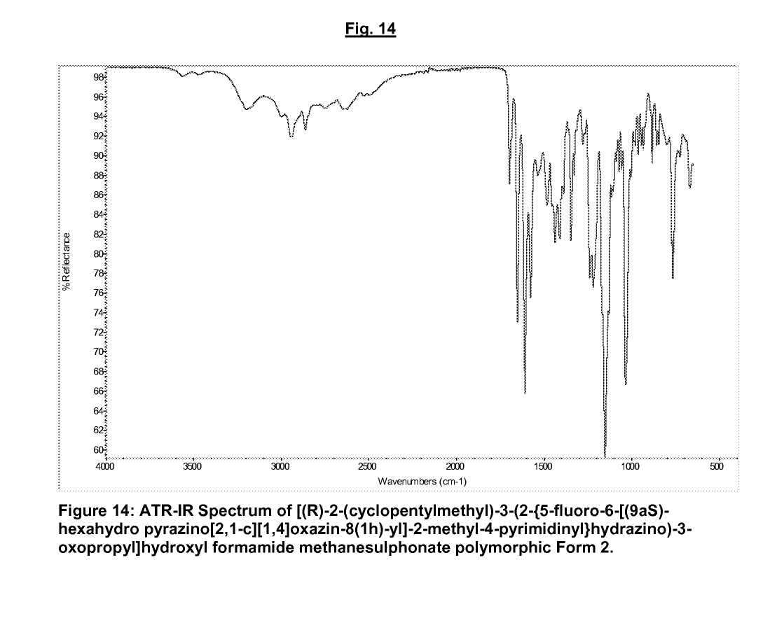

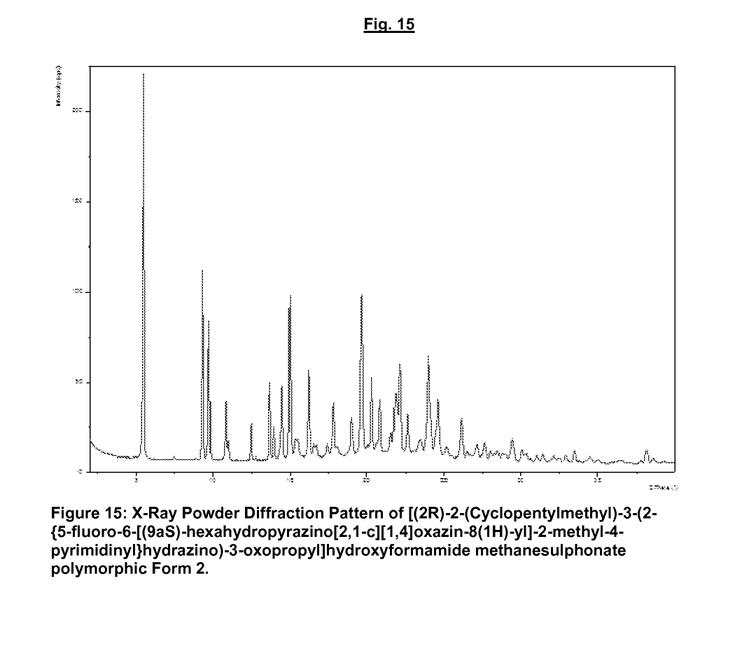

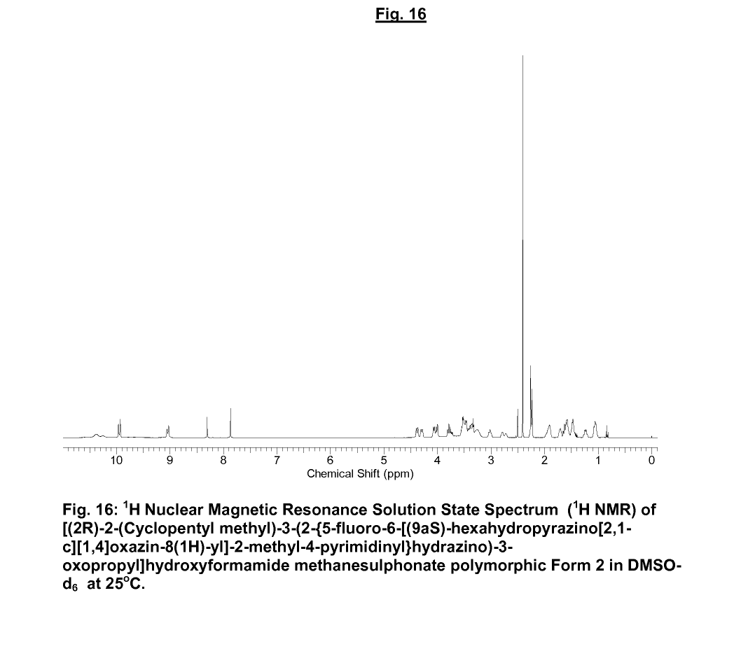

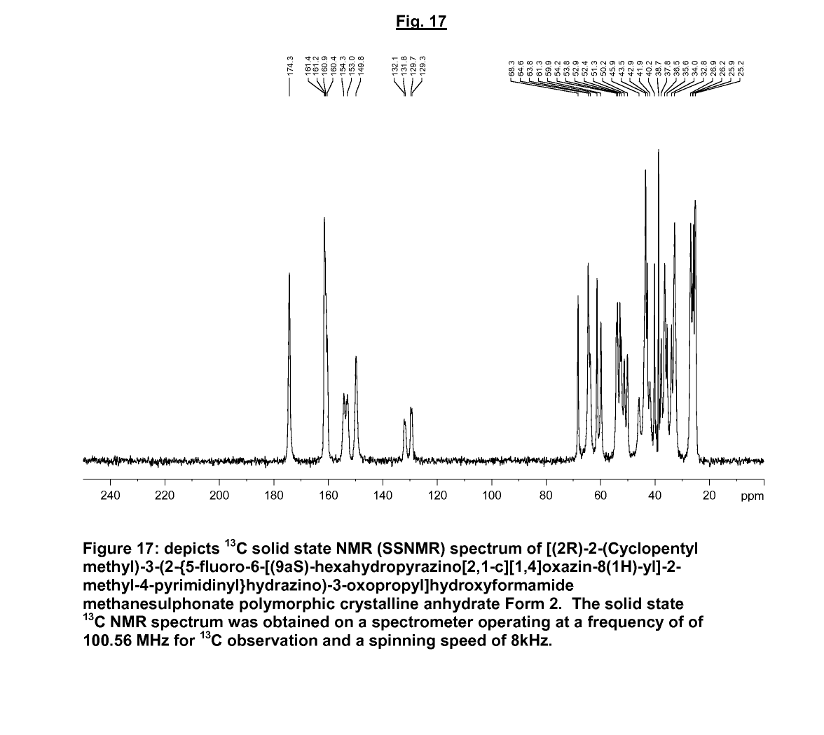

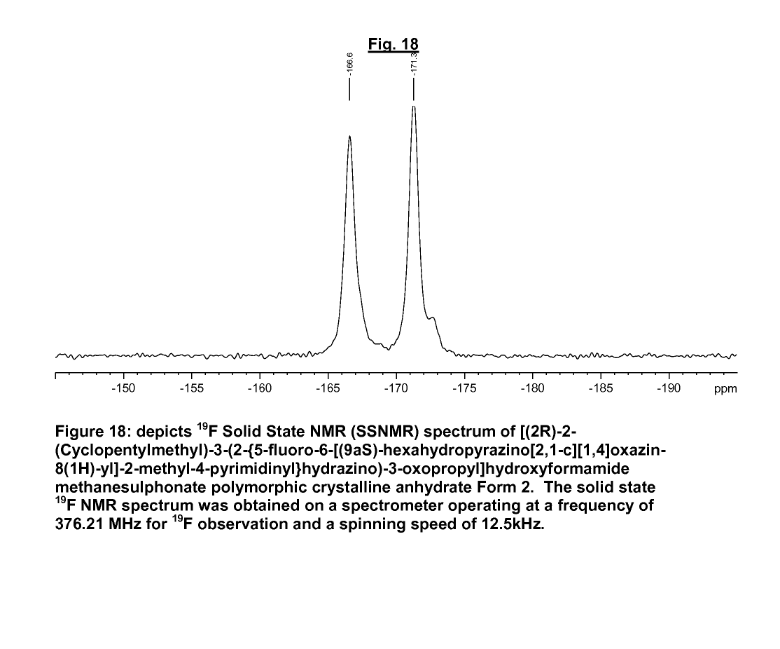

For example, crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 is identified by:
an ATR-IR Spectrum of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 2 (see FIG. 14 and Table 7);
an X-Ray Powder Diffraction Pattern [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 (see FIG. 15 and Table 8);
an X-ray diffraction pattern as shown substantially in FIG. 15, which depicts characteristic peaks from 0° degrees 2-theta (2θ) to 55° degrees 2-theta (2θ) at about 5.5±0.3 (2θ), 9.3±0.3 (2θ), 9.7±0.3 (2θ), 10.8±0.3 (2θ), 13.6±0.3 (2θ), 14.5±0.3 (2θ), 15.0±0.3 (2θ). 16.2±0.3 (2θ), 17.8±0.3 (2θ) and 19.6±0.3 (2θ) (see, Example 11: Form 2)
a ¹H NMR Spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 (see FIG. 16);
a ¹³C NMR and ¹⁹F Solid State Spectrum, respectively, of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 (see, FIGS. 17 and 18); and

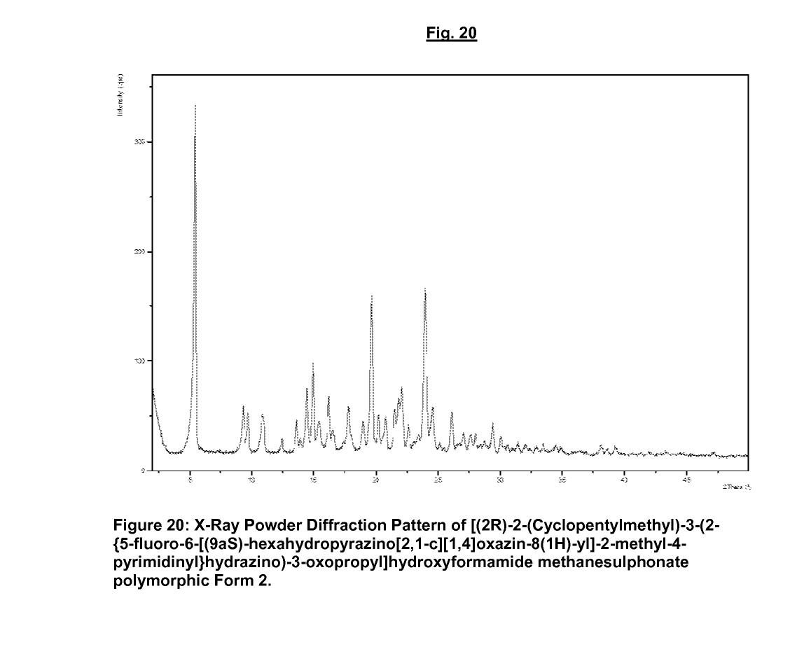

FIG. 20: X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2.

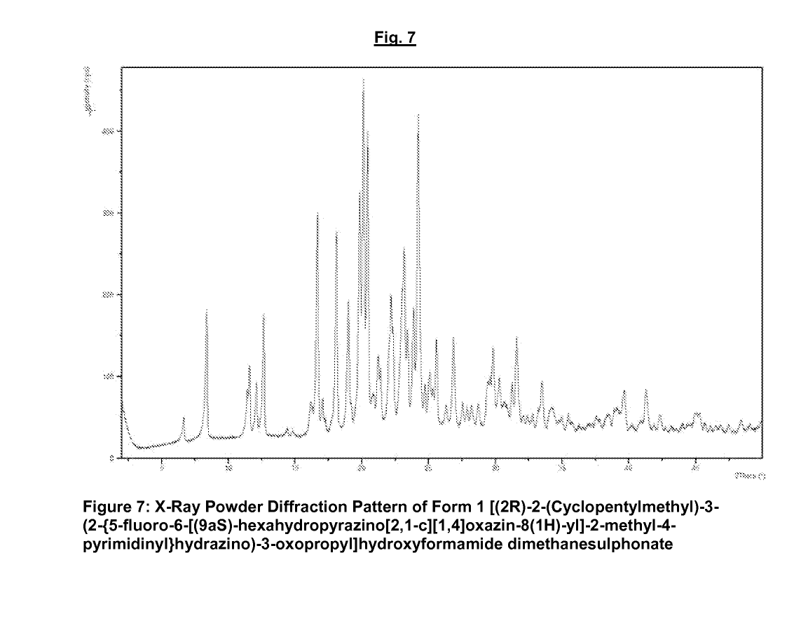

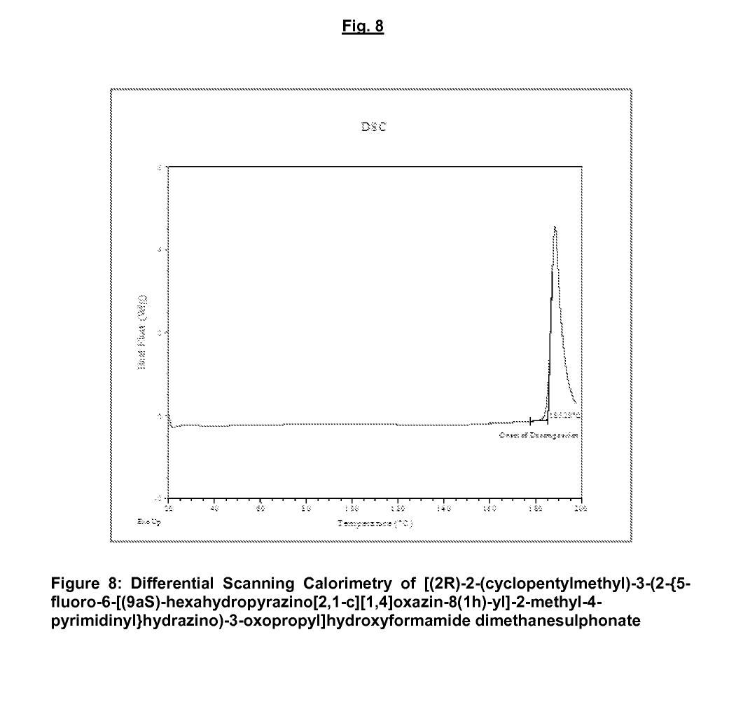

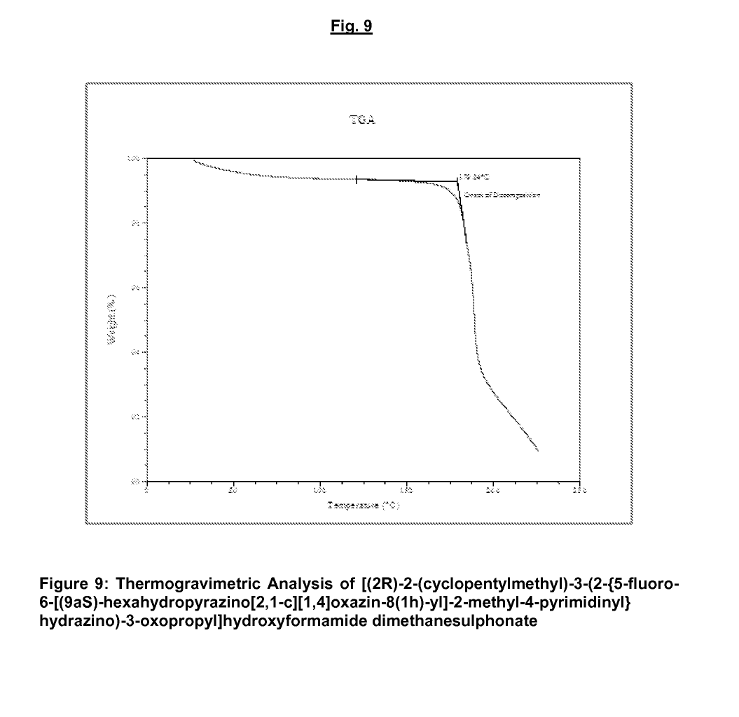

For example, crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate is identified by:
an X-Ray Powder Diffraction Pattern of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate (see, FIG. 7);
a Differential Scanning Calorimetry (DSC) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate (see, FIG. 8); and
a Thermo-Gravimetric Analysis (TGA) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate (see FIG. 9).

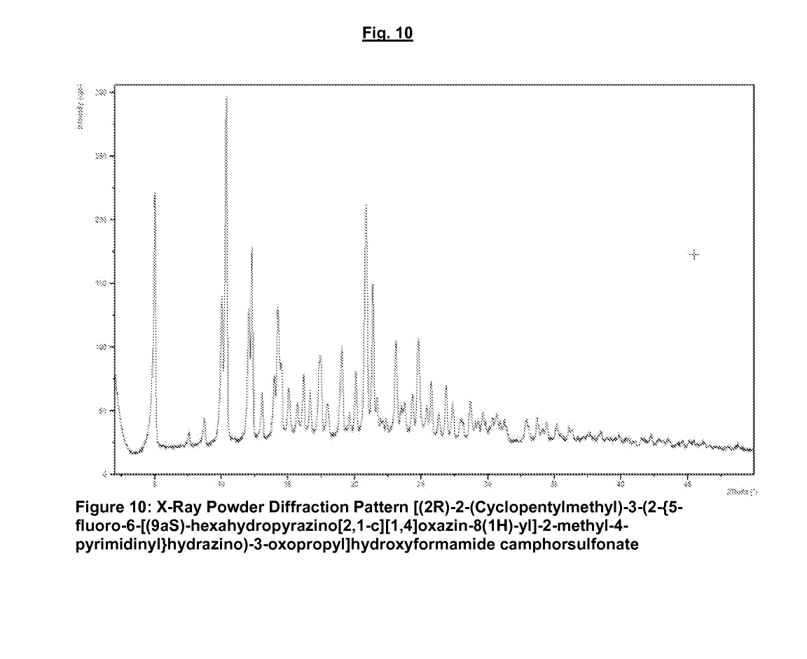

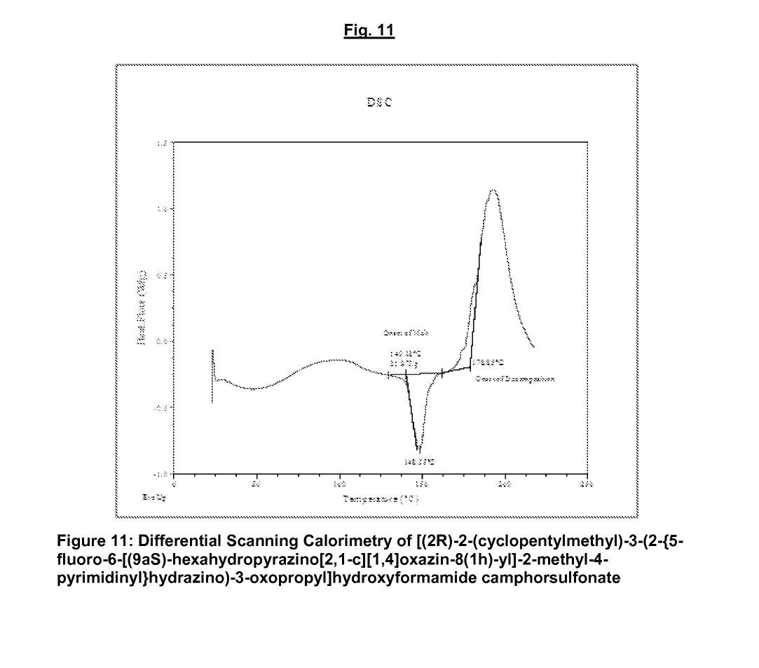

For example, crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulphonate is identified by:
an X-Ray Powder Diffraction Pattern of 1:1 [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate (see FIG. 10); and
a Differential Scanning calorimetry of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide camphorsulfonate (see FIG. 11).

### Salts

In certain embodiments, compounds according to Formula I or a pharmaceutically acceptable salt thereof may contain an acidic functional group. In certain other embodiments, compounds according to Formula I may contain a basic functional group. Thus, the skilled artisan will appreciate that salts of the compounds according to Formula I may be prepared. Indeed, in certain embodiments of the invention, salts of the compounds according to Formula I may be preferred over the respective free base or free acid because, for example, such salts may impart greater stability or solubility to the molecule thereby facilitating formulation into a dosage form.

Because of their potential use in medicine, the salts of the compounds of Formulas (I) are suitably pharmaceutically acceptable salts. Suitable pharmaceutically acceptable salts include those described by Berge, Bighley and Monkhouse J. Pharm. Sci (1977) 66, pp 1-19.

When a compound of the invention is a base (contain a basic moiety), a desired salt form may be prepared by any suitable method known in the art, including treatment of the free base with an inorganic acid, such as hydrochloric acid, hydrobromic acid, sulfuric acid, nitric acid, phosphoric acid, and the like, or with an organic acid, such as acetic acid, trifluoroacetic acid, maleic acid, succinic acid, mandelic acid, fumaric acid, malonic acid, pyruvic acid, oxalic acid, glycolic acid, salicylic acid, pyranosidyl acid, such as glucuronic acid or galacturonic acid, alpha-hydroxy acid, such as citric acid or tartaric acid, amino acid, such as aspartic acid or glutamic acid, aromatic acid, such as benzoic acid or cinnamic acid, sulfonic acid, such as p-toluenesulfonic acid, methanesulfonic acid, ethanesulfonic acid or the like. Examples of pharmaceutically acceptable salts include sulfates, pyrosulfates, bisulfates, sulfites, bisulfites, phosphates, chlorides, bromides, iodides, acetates, propionates, decanoates, caprylates, acrylates, formates, isobutyrates, caproates, heptanoates, propiolates, oxalates, malonates succinates, suberates, sebacates, fumarates, maleates, butyne-1,4-dioates, hexyne-1,6-dioates, benzoates, chlorobenzoates, methyl benzoates, di nitrobenzoates, hydroxybenzoates, methoxybenzoates, phthalates, phenylacetates, phenylpropionates, phenylbutrates, citrates, lactates, γ-hydroxybutyrates, glycollates, tartrates mandelates, and sulfonates, such as xylenesulfonates, methanesulfonates, propanesulfonates, naphthalene-1-sulfonates and naphthalene-2-sulfonates.

Base Salts:

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

If an inventive basic compound is isolated as a salt, the corresponding free base form of that compound may be prepared by any suitable method known to the art, including treatment of the salt with an inorganic or organic base, suitably an inorganic or organic base having a higher pKₐ than the free base form of the compound.

Acid Salts:

Suitable addition salts are formed from acids which form non-toxic salts and examples are hydrochloride, hydrobromide, hydroiodide, sulphate, bisulphate, nitrate, phosphate, hydrogen phosphate, acetate, maleate, malate, fumarate, malonate, lactate, tartrate, citrate, formate, gluconate, succinate, piruvate, oxalate, oxaloacetate, trifluoroacetate, saccharate, benzoate, methanesulphonate, ethanesulphonate, benzenesulphonate, p-toluenesulphonate, methanesulphonic, ethanesulphonic, p-toluenesulphonic, and isethionate.

Certain of the compounds of this invention may form salts with one or more equivalents of an acid (if the compound contains a basic moiety) or a base (if the compound contains an acidic moiety). The present invention includes within its scope all possible stoichiometric and non-stoichiometric salt forms.

When a compound of the invention is an acid (contains an acidic moiety), a desired salt may be prepared by any suitable method known to the art, including treatment of the free acid with an inorganic or organic base, such as an amine (primary, secondary, or tertiary), an alkali metal or alkaline earth metal hydroxide, or the like. Illustrative examples of suitable salts include organic salts derived from amino acids such as glycine and arginine, ammonia, primary, secondary, and tertiary amines, and cyclic amines, such as ethylene diamine, dicyclohexylamine, ethanolamine, piperidine, morpholine, and piperazine, as well as inorganic salts derived from sodium, calcium, potassium, magnesium, manganese, iron, copper, zinc, aluminum, and lithium.

Because the compounds of this invention may contain both acid and base moieties, pharmaceutically acceptable salts may be prepared by treating these compounds with an alkaline reagent or an acid reagent, respectively. Accordingly, this invention also provides for the conversion of one pharmaceutically acceptable salt of a compound of this invention, e.g., a hydrochloride salt, into another pharmaceutically acceptable salt of a compound of this invention, e.g., a sodium salt.

As used herein, the term “pharmaceutically acceptable salts” refers to salts that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, the term “compounds of the invention” means both the compounds according to Formula I and salts thereof, including pharmaceutically acceptable salts. The term “a compound of the invention” also appears herein and refers to both a compound according to Formula I and its salts, including pharmaceutically acceptable salts.

The compounds of the invention may exist in solid or liquid form. In the solid state, the compounds of the invention may exist in crystalline or noncrystalline form, or as a mixture thereof. For compounds of the invention that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

### Solvates

For solvates of the compounds of the invention, or salts thereof, that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water. The invention includes all such solvates.

### Deuterated Compounds

The invention also includes various deuterated forms of the compounds of Formulas (I) or pharmaceutically acceptable salts thereof. Each available hydrogen atom attached to a carbon atom may be independently replaced with a deuterium atom. A person of ordinary skill in the art will know how to synthesize deuterated forms of the compounds of Formulas (I) to (II) of the present invention. For example, deuterated materials, such as alkyl groups may be prepared by conventional techniques (see for example: methyl-d₃-amine available from Aldrich Chemical Co., Milwaukee, Wis., Cat. No. 489, 689-2).

### Isotopes

The subject invention also includes isotopically-labeled compounds which are identical to those recited in Formulas (I) and (II) but for the fact that one or more atoms are replaced by an atom having an atomic mass or mass number different from the atomic mass or mass number most commonly found in nature. Examples of isotopes that can be incorporated into compounds of the invention include isotopes of hydrogen, carbon, nitrogen, oxygen, fluorine, iodine and chlorine such as ³H, ¹¹C, ¹⁴C, ¹⁸F, ¹²³I or ¹²⁵I.

Compounds of the present invention and pharmaceutically acceptable salts of said compounds that contain the aforementioned isotopes and/or other isotopes of other atoms are within the scope of the present invention. Isotopically labeled compounds of the present invention, for example those into which radioactive isotopes such as ³H or ¹⁴C have been incorporated, are useful in drug and/or substrate tissue distribution assays. Tritiated, ie. ³H, and carbon-14, ie. ¹⁴C, isotopes are particularly preferred for their ease of preparation and detectability. ¹¹C and ¹⁸F isotopes are particularly useful in PET (positron emission tomography).

### Purity

Because the compounds of the present invention are intended for use in pharmaceutical compositions it will readily be understood that they are each preferably provided in substantially pure form, for example at least 60% pure, more suitably at least 75% pure and preferably at least 85%, especially at least 98% pure (% are on a weight for weight basis). Impure preparations of the compounds may be used for preparing the more pure forms used in the pharmaceutical compositions.

### Abbreviations and Symbols

In describing the invention, chemical elements are identified in accordance with the Periodic Table of the Elements. Abbreviations and symbols utilized herein are in accordance with the common usage of such abbreviations and symbols by those skilled in the chemical and biological arts. Specifically, the following abbreviations may be used in the examples and throughout the specification:
g (grams); mg (milligrams);
kg (kilograms); μg (micrograms);
L (liters); mL (milliliters);
μL (microliters); psi (pounds per square inch);
M (molar); mM (millimolar);
μM (micromolar); nM (nanomolar);
pM (picomolar); nm (nanometers);
mm (millimeters); wt (weight);
N (Normal); CFU (colony forming units);
I. V. (intravenous); Hz (Hertz);
MHz (megahertz); mol (moles);
mmol (millimoles); RT (room temperature);
min (minutes); h (hours);
b.p. (boiling point); TLC (thin layer chromatography);
Tᵣ (retention time); RP (reverse phase);
MeOH (methanol); i-PrOH (isopropanol);
TEA (triethylamine); TFA (trifluoroacetic acid);
TFAA (trifluoroacetic anhydride); THF (tetrahydrofuran);
DMSO (dimethylsulfoxide); EtOAc (ethyl acetate);
DME (1,2-dimethoxyethane); DCM (dichloromethane);
DCE (dichloroethane); DMF (N,N-dimethylformamide);
DMPU (N,N′-dimethylpropyleneurea); CDl (1,1-carbonyldiimidazole);
IBCF (isobutyl chloroformate); AcOH (acetic acid);
HOAt (1-hydroxy-7-azabenzotriazole);
THP (tetrahydropyran); NMM (N-methylmorpholine);
Pd/C (Palladium on Carbon); MTBE (tert-butyl methyl ether);
HOBT (1-hydroxybenzotriazole); mCPBA (meta-chloroperbenzoic acid;
EDC (1-[3-dimethylamino) propyl]-3-ethylcarbodiimide hydrochloride);
Boc (tert-butyloxycarbonyl); FMOC (9-fluorenylmethoxycarbonyl);
DCC (dicyclohexylcarbodiimide); CBZ (benzyloxycarbonyl);
Ac (acetyl); atm (atmosphere);
TMSE (2-(trimethylsilyl)ethyl); TMS (trimethylsilyl);
TIPS (triisopropylsilyl); TBS (t-butyldimethylsilyl);
DMAP (4-dimethylaminopyridine); BSA (bovine serum albumin)
NAD (nicotinamide adenine dinucleotide);
HPLC (high pressure liquid chromatography);
LC/MS (liquid chromatography/mass spectrometry);
BOP (bis(2-oxo-3-oxazolidinyl)phosphinic chloride);
TBAF (tetra-n-butylammonium fluoride);
HBTU (O-Benzotriazole-1-yl-N,N,N′,N′-tetramethyluroniumhexafluoro phosphate).
HEPES (4-(2-hydroxyethyl)-1-piperazine ethane sulfonic acid);
DPPA (diphenylphosphoryl azide); LAH (Lithium aluminum hydride);
fHNO₃ (fuming HNO₃); NaOMe (sodium methoxide);
EDTA (ethylenediaminetetraacetic acid);
TMEDA (N,N,N′,N′-tetramethyl-1,2-ethanediamine);
NBS (N-bromosuccinimide); DIPEA (diisopropylethylamine);
dppf (1,1′-bis(diphenylphosphino)ferrocene); and
NIS (N-iodsuccinimide).

All references to ether are to diethyl ether and brine refers to a saturated aqueous solution of NaCl.

### Synthetic Schemes and General Methods of Preparation

The present invention also relates to processes for making compounds of Formula (I), or pharmaceutically acceptable salt thereof.

The compounds of Formula (I) or pharmaceutically acceptable salts thereof, may be obtained by using synthetic procedures illustrated in the Schemes below or by drawing on the knowledge of a skilled organic chemist.

The synthesis provided in these Schemes are applicable for producing compounds of the invention having a variety of different functional groups employing appropriate precursors, which are suitably protected if needed, to achieve compatibility with the reactions outlined herein. Subsequent deprotection, where needed, affords compounds of the nature generally disclosed. While the Schemes are shown with compounds, they are illustrative of processes that may be used to make the compounds of the invention.

Intermediates (compounds used in the preparation of the compounds of the invention) may also be present as salts. Thus, in reference to intermediates, the phrase “compound(s) of formula (number)” means a compound having that structural formula or a pharmaceutically acceptable salt thereof.

The present invention also relates to processes for making compounds of Formulas (I) or pharmaceutically acceptable salts thereof. The compounds according to Formulas (I) to (II), respectively, or pharmaceutically acceptable salts thereof, are prepared using conventional organic syntheses.

The compounds of the present invention may be obtained by using synthetic procedures illustrated in Schemes below or by drawing on the knowledge of a skilled organic chemist.

Suitable synthetic routes are depicted below in the following general reaction schemes.

### Compound Preparation

The compounds according to Formula I:

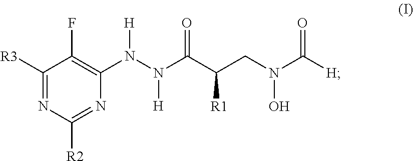

or
pharmaceutically acceptable salts thereof are prepared using conventional organic syntheses. Suitable synthetic routes are depicted below in the following general reaction schemes.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

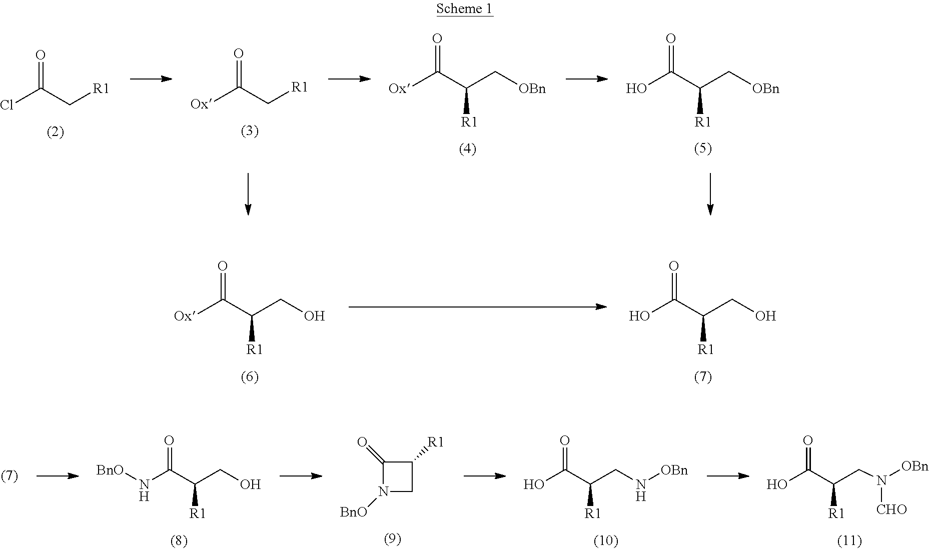

As shown in Scheme 1, (11) can be prepared by reacting an appropriate acid chloride (2) with a chiral agent, such as (S)-(−)-4-benzyl-2-oxazolidinone (Evans' chiral oxazolidinone), in the presence of a base, such as n-butyl lithium, to afford the chiral intermediate (3). Treatment of the compound (3) with a base, such as diisopropylethylamine, in the presence of a chelating agent, such as titanium tetrachloride, in a solvent, such as tetrahydrofuran, followed by addition of an electrophile, such as benzyloxymethylchloride, provides compound (4). Conversion of compound (4) to the corresponding hydroxyacid (7) can be achieved by a sequence comprising oxidative cleavage of the chiral oxazolidinone, using, for example H₂O₂ and lithium hydroxide, to the respective intermediate (5), followed by hydrogenolysis, to afford intermediate (7). Compound (3) can also be converted to intermediate (7) in an alternative two-step procedure. For this transformation, (3) can be treated with a base, such as diisopropylethylamine, in the presence of a chelating agent, such as titanium tetrachloride, in a solvent, such as tetrahydrofuran, followed by addition of trioxane or a suitable alternative formaldehyde equivalent to provide compound (6), which is then submitted to oxidative cleavage of the chiral oxazolidinone, using, for example H₂O₂ and lithium hydroxide, to the respective acid (7).

Coupling of acid (7) with benzyloxyamine in the presence of coupling agents, such as EDC and DMAP, yields the amide (8). This can be cyclized to azetidin-2-one (9) using Mitsunobu conditions. Hydrolysis of the azetidin-2-one (9), using for example lithium hydroxide in an appropriate solvent, gives the corresponding acid (10). Conversion of compound (10) to product (11) can be achieved using an appropriate formylating agent, such as formic acid/acetic anhydride or methyl formate, in neat reagents or in an appropriate solvent, such as dichloromethane.

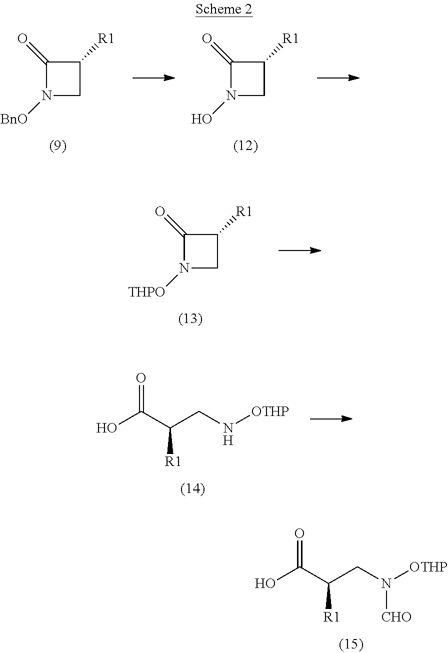

As shown in Scheme 2, THP-protected intermediate (15) can be prepared by hydrogenation of azetidin-2-one (9) using a catalyst, such as 10% Pd/C, in an appropriate solvent, such as ethanol to provide (12). Treatment of (12) with dihydropyran under acid catalysis, such as pyridinium p-toluenesulfonate, in an appropriate solvent, such as methylene chloride, provides THP-protected azetidin-2-one (13). Hydrolysis of azetidin-2-one (13), using for example lithium hydroxide in an appropriate solvent, gives the corresponding acid (14). Conversion of compound (14) to the product (15) can be achieved using an appropriate formylating agent, such as formic acid/acetic anhydride or methyl formate, in neat reagents or in an appropriate solvent, such as dichloromethane. Conversion of compound (14) to product (15) can also be accomplished using 5-methyl-2-thioxo-[1,3,4]thiadiazole-3-carbaldehyde (Yazawa, Hisatoyo; Goto, Shunsuke; Tetrahedron Lett. 26; 31; 1985; 3703-3706) as a formylating agent in an appropriate solvent, such as acetone.

Intermediate (15) can also be prepared according to literature procedures [Bracken, Bushell, Dean, Francavilla, Jain, Lee, Seepersaud, Shu, Sundram, Yuan; PCT Int. Appl. (2006), WO 2006127576 A2].

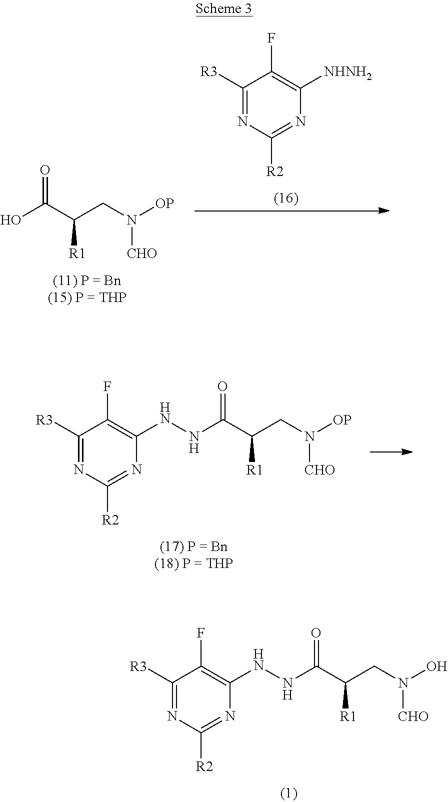

As shown in Scheme 3, coupling of the chiral acid (11 or 15) with the pyrimidinyl hydrazine (16, R2=alkyl, halo, H), using conditions such as EDC-HOAt-NMM, provides the hydrazide (17 or 18). Final deprotection (hydrogenolysis using a catalyst, such as 10% Pd/C, in an appropriate solvent, such as ethanol, in the case that P is Bn; treatment with 80% acetic acid-water at room temperature or 40° C. in the case that P is THP) gives the final desired compounds (1), where R2=alkyl, halo, H.

Hydrazines of general structure (16) may be prepared according to literature methods by those skilled in the art. The following examples of specific structures of hydrazines (16) and the synthetic methods used to generate them are merely illustrative and are not to be construed as a limitation of the scope of the present invention.

Hydrazines (24) where R2 is alkyl and R3 is an amino group (R4R5N) may be prepared from the appropriate precursors as shown in Scheme 4.

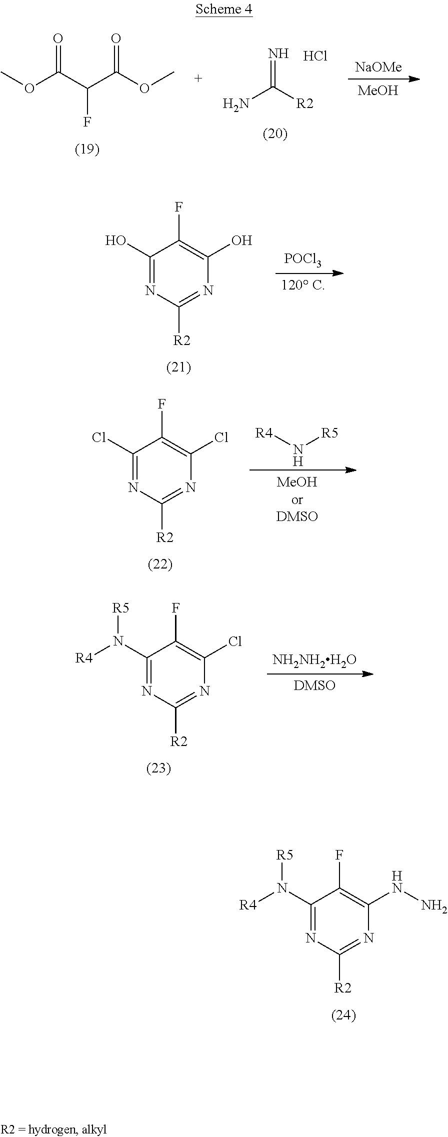

As shown in Scheme 4, hydrazine (24) when R2 is hydrogen or alkyl can be prepared from the condensation of commercially-available fluoromalonate (19) and the appropriate amidine (20) under basic conditions to provide pyrimidinone (21). Amidines (20) are commercially available or may be prepared according to literature methods by those skilled in the art. Treatment of pyrimidinone (21) with POCl₃ provides dichloropyrimidine (22). Treatment of dichloropyrimidine (22) with the desired amine R4R5NH at room temperature in an appropriate solvent, such as methanol or DMSO, followed by further treatment with hydrazine monohydrate, in an appropriate solvent, such as DMSO, usually with heating, then provides the desired product (24) where R2 is hydrogen or alkyl.

Hydrazines of formula (30) [(16) in which R2=chloro] may be prepared as shown in either Schemes 5 or 6.

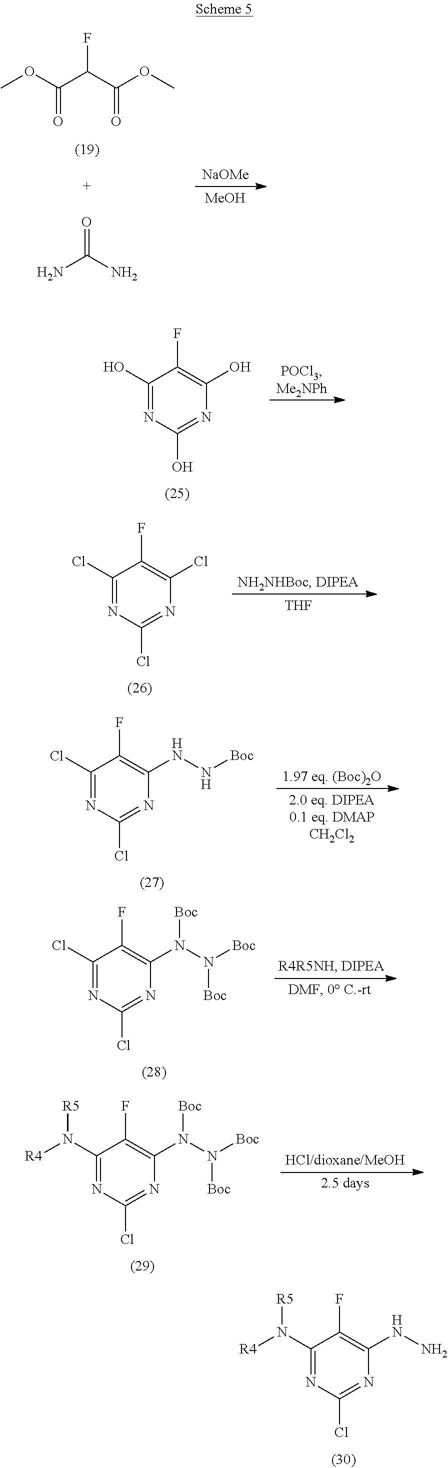

Condensation of commercially-available fluoromalonate (19) and urea under basic conditions provides pyrimidinone (25). Treatment of pyrimidinone (25) with POCl₃ provides trichloropyrimidine (26). Treatment of trichloropyrimidine (26) with Boc-protected hydrazine and diisopropylethylamine at room temperature in an appropriate solvent, such as THF, provides intermediate (27). Further treatment with di-t-butyldicarbonate in the presence of diisopropylethylamine and DMAP, in an appropriate solvent, such as methylene chloride, then provides the desired tri-Boc-protected product (28). Treatment of (28) with amine R4R5NH, in an appropriate solvent such as DMF, provides pyrimidine (29), and deprotection of (29) under acidic conditions, followed by a basic workup, provides the desired hydrazines (30).

Alternatively, hydrazines of formula (30) may be prepared as shown in Scheme 6.

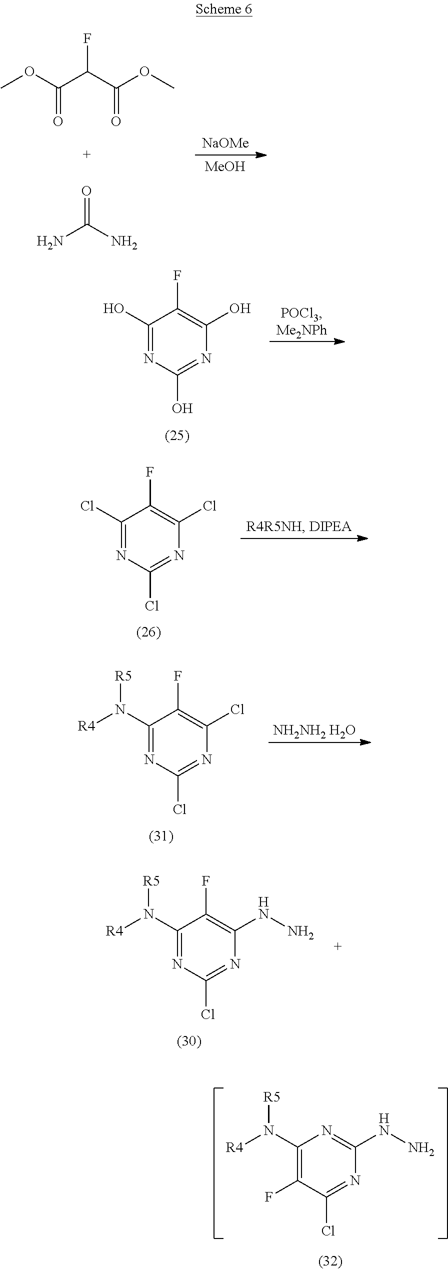

Treatment of trichloropyrimidine (26) with the desired amine R4R5NH at room temperature in an appropriate solvent, such as DMSO, followed by further treatment with hydrazine monohydrate and heating, provides the desired product (30), as well as the regioisomeric product (32). The two regioisomers can usually be separated chromatographically, such as by HPLC.

Final compounds (1) where R2 is thiomethyl or methoxy can be prepared as shown in Scheme 7.

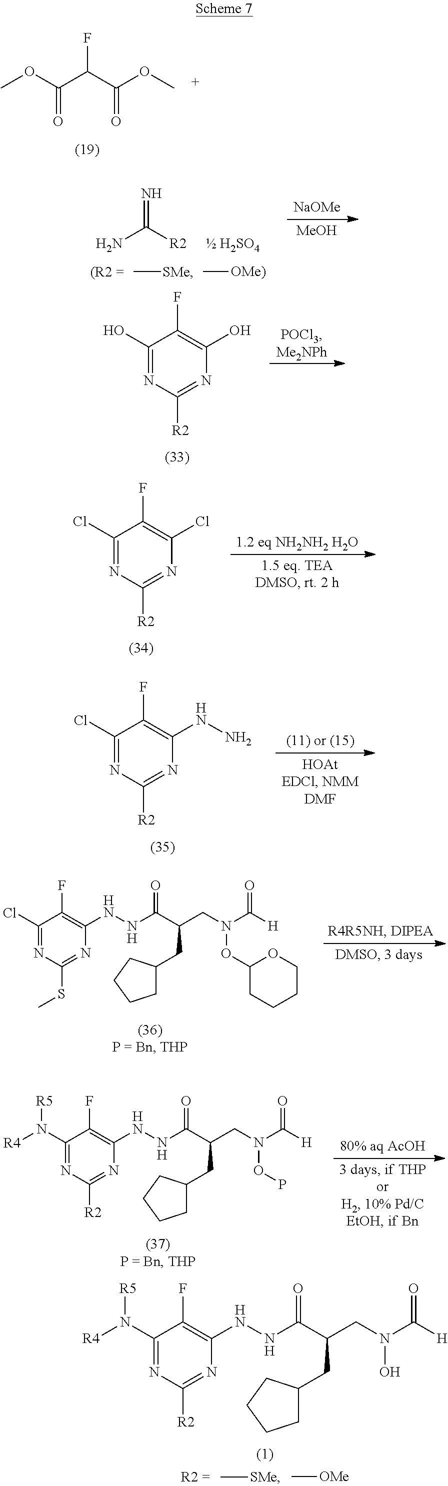

Condensation of commercially-available fluoromalonate (19) and either O-methylisourea hemisulfate or S-methylisothiourea hemisulfate under basic conditions provides pyrimidinone (33), in which R2 is methoxy or thiomethyl, respectively. Treatment of pyrimidinone (33) with POCl₃ provides dichloropyrimidine (34). Treatment of dichloropyrimidine (34) with hydrazine monohydrate, in an appropriate solvent, such as methanol, provides pyrimidinylhydrazine (35), which is then coupled to acid (11) or (15) using conditions such as EDC-HOAt-NMM to provide intermediate (36). Addition of R4R5NH to intermediate (36) provides either the O-Bn-protected or O-THP-protected product (37). Final deprotection by either hydrogenolysis using a catalyst such as 10% Pd/C, in an appropriate solvent, such as ethanol, in the case that P is Bn; or treatment with 80% acetic acid-water at room temperature or 40° C. in the case that P is THP, gives the final desired compounds (1) when R2 is methoxy or thiomethyl, respectively.

Amines R4R5NH may be purchased from available commercial sources, prepared according to literature methods by those skilled in the art, or prepared as disclosed in the examples herein.

### Pharmaceutical Compositions, Dosage Forms, and Regimens

The present invention relates to pharmaceutical compositions comprised of novel compounds of Formula (I) or pharmaceutically acceptable salts thereof and at least one pharmaceutically acceptable excipient(s).

Suitable compounds for use in pharmaceutical compositions of the present invention, may include, but are not limited to:
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate Forms 1 and 2;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate); or
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

The compounds of the invention will normally, but not necessarily, be formulated into pharmaceutical compositions prior to administration to a patient.

Accordingly, in another aspect, the invention is directed to pharmaceutical compositions comprising a compound of Formula (I) or pharmaceutically acceptable salts thereof of the present invention and one or more pharmaceutically acceptable excipient(s). In particular, the present invention also may relate to a pharmaceutical composition or formulation, which comprises a compound as defined by Formula (I) or a pharmaceutically acceptable salt thereof, and at least one pharmaceutically acceptable excipient(s), and optionally one or more other therapeutic ingredients.

The pharmaceutical compositions of the invention may be prepared and packaged in bulk form wherein a safe and effective amount of a compound of the invention can be extracted and then given to the patient such as with powders or syrups. Alternatively, the pharmaceutical compositions of the invention may be prepared and packaged in unit dosage form wherein each physically discrete unit contains a safe and effective amount of a compound of the invention. When prepared in unit dosage form, the pharmaceutical compositions of the invention typically contain from 25 mg to 1.5 g, suitably 100 to 500 mg, of compound of the invention.

The pharmaceutical compositions of the invention typically contain one compound of the invention. However, in certain embodiments, the pharmaceutical compositions of the invention may contain more than one compound of the invention. For example, in certain embodiments the pharmaceutical compositions of the invention may contain two compounds of the invention. In addition, the pharmaceutical compositions of the invention may optionally further comprise one or more additional pharmaceutically active compounds.

As used herein, “pharmaceutically acceptable excipient” means a pharmaceutically acceptable material, composition or vehicle that, for example, are involved in giving form or consistency to the pharmaceutical composition. Each excipient must be compatible with the other ingredients of the pharmaceutical composition when commingled such that interactions which would substantially reduce the efficacy of the compound of the invention when administered to a patient and interactions which would result in pharmaceutical compositions that are not pharmaceutically acceptable are avoided. In addition, each excipient must of course be of sufficiently high purity to render it pharmaceutically-acceptable.

The compound of the invention and the pharmaceutically acceptable excipient or excipients will typically be formulated into a dosage form adapted for administration to the patient by the desired route of administration. For example, dosage forms include those adapted for (1) oral administration such as tablets, capsules, caplets, pills, troches, powders, syrups, elixers, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as dry powders, aerosols, suspensions, and solutions; and (6) topical administration such as creams, ointments, lotions, solutions, pastes, sprays, foams, and gels.

Suitable pharmaceutically-acceptable excipients will vary depending upon the particular dosage form chosen. In addition, suitable pharmaceutically acceptable excipients may be chosen for a particular function that they may serve in the composition.

For example, certain pharmaceutically-acceptable excipients may be chosen for their ability to facilitate the production of uniform dosage forms. Certain pharmaceutically acceptable excipients may be chosen for their ability to facilitate the production of stable dosage forms. Certain pharmaceutically-acceptable excipients may be chosen for their ability to facilitate the carrying or transporting of the compound or compounds of the invention once administered to the patient from one organ, or portion of the body, to another organ, or another portion of the body. Certain pharmaceutically acceptable excipients may be chosen for their ability to enhance patient compliance. Moreover, pharmaceutical compositions, formulations, dosage forms, and the like, etc. may conveniently be presented in unit dosage form and may be prepared by any of the methods well known in the art of pharmacy.

All methods include the step of bringing the active ingredient into association with the carrier which constitutes one or more accessory ingredients. In general the formulations are prepared by uniformly and intimately bringing into association the active ingredient with liquid carriers or finely divided solid carriers or both and then, if necessary, shaping the product into the desired formulation.

Suitable pharmaceutically acceptable excipients include the following types of excipients: Diluents, fillers, binders, disintegrants, lubricants, glidants, granulating agents, coating agents, wetting agents, solvents, co-solvents, suspending agents, emulsifiers, sweetners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically acceptable excipients may serve more than one function and may serve alternative functions depending on how much of the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

The compounds of the invention and the pharmaceutically-acceptable excipient or excipients will typically be formulated into a dosage form adapted for administration to the patient by the desired route of administration.

With regard to the present invention, conventional dosage forms include those adapted for (1) oral administration such as tablets, capsules, caplets, pills, troches, powders, syrups, elixirs, suspensions, solutions, emulsions, sachets, and cachets; (2) parenteral administration such as sterile solutions, suspensions, and powders for reconstitution; (3) transdermal administration such as transdermal patches; (4) rectal administration such as suppositories; (5) inhalation such as aerosols and solutions; (6) topical administration such as creams, ointments, lotions, solutions, pastes, sprays, foams, and gels.

In general, pharmaceutical compositions of the present invention are prepared using conventional materials and techniques, such as mixing, blending and the like.

The term “active agent” is defined for purposes of the present invention as any chemical substance or composition of the present invention, which can be delivered from the device into an environment of use to obtain a desired result.

The percentage of the compound in compositions can, of course, be varied as the amount of active in such therapeutically useful compositions is such that a suitable dosage will be obtained.

In one aspect, the present invention is directed to a pharmaceutical composition comprising a compound of Formula (I) or a pharmaceutically acceptable salt thereof and one or more pharmaceutically acceptable excipients.

In another aspect, the present invention is directed to a pharmaceutical composition comprising a compound of Formula (II) or a pharmaceutically acceptable salt thereof and one or more pharmaceutically acceptable excipients.

In another aspect, the present invention is directed to a pharmaceutical composition comprising a compound or compound species or a pharmaceutically acceptable salt thereof of the present invention as defined herein and one or more pharmaceutically acceptable excipients.

It will be appreciated that the actual preferred dosages of the compounds being used in the compositions of this invention will vary according to the particular composition formulated, the mode of administration, the particular site of administration and the host being treated.

The active compounds of the present invention may be orally administered, for example, with an inert diluent, or with an assimilable edible carrier, or they can be enclosed in hard or soft shell capsules, or they can be compressed into tablets, or they can be incorporated directly with the food of the diet, etc.

In one aspect, the invention is directed to a solid oral dosage form such as a tablet or capsule comprising a safe and effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmelose, alginic acid, and sodium carboxymethyl cellulose. The oral solid dosage form may further comprise a lubricant. Suitable lubricants include stearic acid, magnesuim stearate, calcium stearate, and talc.

Where appropriate, dosage unit formulations for oral administration can be microencapsulated. The composition can also be prepared to prolong or sustain the release as for example by coating or embedding particulate material in polymers, wax or the like.

The compounds of the invention may also be coupled with soluble polymers as targetable drug carriers. Such polymers can include polyvinylpyrrolidone, pyran copolymer, polyhydroxypropylmethacrylamide-phenol, polyhydroxyethylaspartamidephenol, or polyethyleneoxidepolylysine substituted with palmitoyl residues. Furthermore, the compounds of the invention may be coupled to a class of biodegradable polymers useful in achieving controlled release of a drug, for example, polylactic acid, polepsilon caprolactone, polyhydroxy butyric acid, polyorthoesters, polyacetals, polydihydropyrans, polycyanoacrylates and cross-linked or amphipathic block copolymers of hydrogels.

In another aspect, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to parenteral administration, that is by intravenous, intramuscular, subcutaneous intranasal, intrarectal, intravaginal, intraperitoneal administration, intrasternal injection or infusion techniques. The subcutaneous and intramuscular forms of parenteral administration are generally preferred. In one aspect, the compositions are administered parenterally, most suitably intravenously. Appropriate dosage forms for such administration may be prepared by conventional techniques.

Pharmaceutical compositions of the invention can be formulated so as to allow a compound of the present invention to be bioavailable upon administration of the composition to a subject.

Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules, vials, pouches and the like and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

The pharmaceutical composition can be in the form of a liquid, e.g., an elixir, syrup, solution, emulsion or suspension. The liquid can be useful for oral administration or for delivery by injection. When intended for a composition for administration by injection, one or more of a surfactant, preservative, wetting agent, dispersing agent, suspending agent, buffer, stabilizer and isotonic agent can also be included.

The liquid compositions of the invention, whether they are solutions, suspensions or other like form, can also include one or more of the following: sterile diluents such as water for injection, saline solution, preferably physiological saline, Ringer's solution, isotonic sodium chloride, fixed oils such as synthetic mono or digylcerides which can serve as the solvent or suspending medium, polyethylene glycols, glycerin, cyclodextrin, propylene glycol or other solvents; antibacterial agents such as benzyl alcohol or methyl paraben; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. A parenteral composition can be enclosed in ampoule, a disposable syringe or a multiple-dose vial made of glass, plastic or other material. Physiological saline is a preferred adjuvant. An injectable composition is preferably sterile.

The amount of a compound of the present invention that is effective in the treatment of a particular disorder or condition will depend on the nature of the disorder or condition, and can be determined by standard clinical techniques. In addition, in vitro or in vivo assays can optionally be employed to help identify optimal dosage ranges. The precise dose to be employed in the compositions will also depend on the route of administration, and the seriousness of the disease or disorder, and should be decided according to the judgment of the practitioner and each patient's circumstances.

In another aspect, the present invention is directed to a pharmaceutical composition which comprises [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate.

In another aspect, the present invention is directed to a pharmaceutical composition which comprises [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 or Form 2, respectively, which is a crystalline anhydrate or crystalline anhydrous form, a hydrate, or a mixture thereof.

In another aspect, the present invention is directed to a pharmaceutical composition which comprises [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 which is a crystalline anhydrate or crystalline anhydrous form.

In another aspect, the present invention is directed to a pharmaceutical composition which comprises [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 which is a crystalline anhydrate or crystalline anhydrous form.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide, or a pharmaceutically acceptable salt thereof, and at least one pharmaceutically acceptable excipient.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate and at least one pharmaceutically acceptable excipient.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 and at least one pharmaceutically acceptable excipient.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 and at least one pharmaceutically acceptable excipient.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate and at least one pharmaceutically acceptable excipient.

In another aspect, the present invention is directed to a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulphonate and at least one pharmaceutically acceptable excipient.

### Administration

Treatment regimen for the administration of compounds of Formula (I), or pharmaceutically acceptable salts thereof or corresponding pharmaceutical compositions of the present inay vention also may be determined readily by those with ordinary skill in art.

In one aspect, the present invention relates to administration of compounds of Formula (I) or pharmaceutically acceptable salts thereof or corresponding pharmaceutical compositions thereof.

Suitable compounds for administration, alone, or in a pharmaceutical composition of the present invention, include, but are not limited to:
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesuphonate Forms 1 and 2, respectively;
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., also identified as [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate); or
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate and the like.

The quantity of the compound, pharmaceutical composition, or dosage form of the present invention administered may vary over a wide range to provide in a unit dosage in an effective amount based upon the body weight of the patient per day to achieve the desired effect and as based upon the mode of administration.

The scope of the present invention includes all compounds, pharmaceutical compositions, or controlled-release formulations or dosage forms, which is contained in an amount effective to achieve its intended purpose. While individual needs vary, determination of optimal ranges of effective amounts of each component is within the skill of the art.

Compounds of Formula (I) or pharmaceutically acceptable salts thereof or corresponding pharmaceutical compositions of the present invention may be administered by any suitable route of administration, including both systemic administration and topical administration. Systemic administration includes oral administration, parenteral administration, transdermal administration, rectal administration, and administration by inhalation.

Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. In one aspect, pharmaceutical compositions, formulations, dosages, dosage forms or dosing regimens of the present invention are adapted for administration by inhalation.

Topical administration includes application to the skin as well as intraocular, intravaginal, and intranasal administration.

Compounds of Formula (I) or pharmaceutically acceptable salts thereof or corresponding pharmaceutical compositions of the present invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect.

Suitable dosing regimens for compounds of Formula (I) or pharmaceutically acceptable salts thereof or corresponding pharmaceutical compositions of the present invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient being treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

In another aspect, the invention is directed to a liquid oral dosage form. Oral liquids such as solution, syrups and elixirs can be prepared in dosage unit form so that a given quantity contains a predetermined amount of a compound of the invention. Syrups can be prepared by dissolving the compound of the invention in a suitably flavored aqueous solution, while elixirs are prepared through the use of a non-toxic alcoholic vehicle. Suspensions can be formulated by dispersing the compound of the invention in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

In another aspect, the invention is directed to parenteral administration. Pharmaceutical compositions adapted for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

Typical daily dosages may vary depending upon the particular route of administration chosen. Typical daily dosages for oral administration, to a human weighing approximately 70 kg, would range from 7 mg to 7 g, suitably 3.5 mg to 3.5 g of a compound of the invention a day.

Compounds of Formula (I) or pharmaceutically acceptable salts thereof or corresponding pharmaceutical compositions of the present invention may be administered parenterally or orally as an injecting agent, capsules, tablets, and granules, and preferably, administered as an injecting agent. Amounts to be administered may usually be, per 1 kg of body weight of a patient or animal, about 0.1 to 100 mg/day, preferably, about 0.5 to 50 mg/day, if desired, divided into 2-4 times per day. Carriers when used as an injecting agent is for example, distilled water, saline and the like, and base and the like may be used for pH adjustment. When used as capsules, granules or tablets, carriers may be known excipients (e.g., starch, lactose, sucrose, calcium carbonate, calcium phosphate and the like), binders (e.g., starch, acacia gum, carboxymethyl cellulose, hydroxypropyl cellulose, crystalline cellulose, and the like), lubricants (e.g., magnesium stearate, talc and the like), and the like.

For all methods of use disclosed herein for the compounds of Formulas (I) to (II), the daily oral dosage regimen will preferably be from about 0.05 to about 80 mg/kg of total body weight, preferably from about 0.1 to 30 mg/kg, more preferably from about 0.5 mg to 15 mg/kg, administered in one or more daily doses. For example, the daily parenteral dosage regimen about 0.1 to about 80 mg/kg of total body weight, preferably from about 0.2 to about 30 mg/kg, and more preferably from about 0.5 mg to 15 mg/kg, administered in one or more daily doses. The daily topical dosage regimen will preferably be from 0.01 mg to 150 mg, administered one to four times daily. The daily inhalation dosage regimen will preferably be from about 0.05 microgram/kg to about 5 mg/kg per day, or from about 0.2 microgram/kg to about 20 microgram/kg, administered in one or more daily doses.

It will also be recognized by one of skill in the art that the optimal quantity and spacing of individual dosages of a compound of Formulas (I) to (II), respectively, or a pharmaceutically acceptable salt thereof will be determined by the nature and extent of the condition being treated, the form, route and site of administration, and the particular patient being treated, and that such optimums can be determined by conventional techniques. It will also be appreciated by one of skill in the art that the optimal course of treatment, i.e., the number of doses of a compound of Formulas (I) to (II), respectively, or a pharmaceutically acceptable salt thereof given per day for a defined number of days, can be ascertained by those skilled in the art using conventional course of treatment determination tests.

The amount of a compounds of Formula (I) or pharmaceutically acceptable salts thereof or corresponding pharmaceutical compositions of the present invention which is required to achieve a therapeutic effect will, of course, vary with the particular compound, the route of administration, the subject under treatment, and the particular disorder or disease being treated.

Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient to be treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Additionally, the compounds of the present invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention also provides a compound of the invention for use in medical therapy, particularly in bacterial infections. Thus, in a further aspect, the invention is directed to the use of a compound according to Formula I or a pharmaceutically-acceptable salt thereof in the preparation of a medicament for the treatment of bacterial infections.

In another aspect, the present invention is directed to a [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide, or a pharmaceutically acceptable salt thereof, may be formulated for oral administration, suitably in a liquid or tablet form, or for patenteral administration.

In another aspect, the present invention is directed to a pharmaceutical composition or formulation as defined above, where each is formulated for intravenous (iv) administration.

### Methods of Use

The present invention also relates to methods for treating bacterial infections, which comprises administering to a subject in need thereof an effective amount of a compound of Formula (I), or a pharmaceutically acceptable salt thereof.

The compounds of the invention are inhibitors of microbial peptide deformylase (PDF) and are, therefore, capable of preventing bacterial growth. These compounds are potentially useful in the treatment of infectious diseases wherein the underlying pathology is (at least in part) attributable to (i.e. caused by) a variety of prokaryotic organisms.

Examples include, but are not limited to, Gram positive and Gram negative aerobic and anaerobic bacteria from the genera Streptococcus, e.g. S. pneumoniae and S. pyogenes, Staphylococcus, e.g. S. aureus, S. epidermidis, and S. saprophyticus, Moraxella, e.g. M. catarrhalis, Haemophilus, e.g. H. influenzae, Neisseria, Mycoplasma, e.g. M. pneumoniae, Legionella, e.g. L. pneumophila, Chlamydia, e.g. C. pneumoniae, Bacteroides, Clostridium, Fusobacterium, Propionibacterium, and Peptostreptococcus.

Suitably the compounds of the present invention may be useful in the treatment of bacterial infections caused by Streptococcus, more suitably S. pneumoniae or S. pyogenes.

Suitably the compounds of the present invention may be useful in the treatment of bacterial infections caused by Staphylococcus, more suitably S. aureus, S. epidermidis, or S. saprophyticus.

Suitably the compounds of the present invention may be useful in the treatment of bacterial infections caused by Moraxella, more suitably M. catarrhalis.

Suitably the compounds of the present invention may be useful in the treatment of bacterial infections caused by Haemophilus, more suitably H. influenzae.

Suitably the compounds of the present invention may be useful in the treatment of bacterial infections caused by Neisseria.

Suitably the compounds of the present invention may be useful in the treatment of bacterial infections caused by Mycoplasma, more suitably M. pneumoniae.

Suitably the compounds of the present invention may be useful in the treatment of bacterial infections caused by Legionella, more suitably L. pneumophila.

Suitably the compounds of the present invention may be useful in the treatment of bacterial infections caused by Chlamydia, more suitably C. pneumoniae.

Suitably the compounds of the present invention may be useful in the treatment of bacterial infections caused by Bacteroides.

Suitably the compounds of the present invention may be useful in the treatment of bacterial infections caused by Clostridium.

Suitably the compounds of the present invention may be useful in the treatment of bacterial infections caused by Fusobacterium.

Suitably the compounds of the present invention may be useful in the treatment of bacterial infections caused by Propionibacterium.

Suitably the compounds of the present invention may be useful in the treatment of bacterial infections caused by Peptostreptococcus.

The compounds of the invention may also be useful in the treatment of bacterial infections caused by bacteria that are resistant to β-lactam, quinolone, macrolides, ketolides, glycopeptide, and oxazolidinone classes of antibiotics. Such drug resistant bacterial infections include, but are not limited to, penicillin, macrolide or levofloxacin resistant S. pneumoniae; methicillin or macrolide resistant, and vancomycin intermediate S. aureus; methicillin resistant S. epidermidis; and oxazolidinone resistant S. aureus.

The compounds of the invention may be used to treat a bacterial infection in mammals, specifically humans. Such infections include, but are not limited to, ear infections, sinusitis, upper and lower respiratory tract infections, genital infections, skin and soft tissue infections, and bacterial endocarditis. The compounds of the invention may also be used to prevent a bacterial infection in mammals, specifically humans, such as a bacterial infection that may result from medical or dental procedures.

Suitably the compounds of the invention may be used to treat ear infections.

Suitably the compounds of the invention may be used to treat sinusitis.

Suitably the compounds of the invention may be used to treat upper and lower respiratory tract infections.

Suitably the compounds of the invention may be used to treat genital infections.

Suitably the compounds of the invention may be used to treat skin and soft tissue infections.

Suitably the compounds of the invention may be used to treat bacterial endocarditis.

As used herein, “infectious disease” refers to any disease characterized by the presence of a microbial infection, such as a bacterial infection.

As used herein, “treat” in reference to a condition means: (1) to ameliorate or prevent the condition or one or more of the biological manifestations of the condition, (2) to interfere with (a) one or more points in the biological cascade that leads to or is responsible for the condition or (b) one or more of the biological manifestations of the condition, (3) to alleviate one or more of the symptoms or effects associated with the condition, or (4) to slow the progression of the condition or one or more of the biological manifestations of the condition.

As indicated above “treatment” of a condition includes prevention of the condition. The skilled artisan will appreciate that “prevention” is not an absolute term. In medicine, “prevention” is understood to refer to the prophylactic administration of a drug to substantially diminish the likelihood or severity of a condition or biological manifestation thereof, or to delay the onset of such condition or biological manifestation thereof.

As used herein, “effective amount” in reference to a compound of the invention means an amount of the compound sufficient to treat the patient's condition, but low enough to avoid serious side effects (at a reasonable benefit/risk ratio) within the scope of sound medical judgment. An effective amount of a compound will vary with the particular compound chosen (e.g., consider the potency, efficacy, and half-life of the compound); the route of administration chosen; the condition being treated; the severity of the condition being treated; the age, size, weight, and physical condition of the patient being treated; the medical history of the patient being treated; the duration of the treatment; the nature of concurrent therapy; the desired therapeutic effect; and like factors, and can be routinely determined by the skilled artisan.

As used herein, “patient” refers to a human or other mammal.

The compounds of the invention may be administered by any suitable route of administration, including both systemic administration and topical administration. Systemic administration includes oral administration, parenteral administration, transdermal administration, rectal administration, and administration by inhalation. Parenteral administration refers to routes of administration other than enteral, transdermal, or by inhalation, and is typically by injection or infusion. Parenteral administration includes intravenous, intramuscular, and subcutaneous injection or infusion. Inhalation refers to administration into the patient's lungs whether inhaled through the mouth or through the nasal passages. Topical administration includes application to the skin as well as intraocular, intravaginal, and intranasal administration.

The compounds of the invention may be administered once or according to a dosing regimen wherein a number of doses are administered at varying intervals of time for a given period of time. For example, doses may be administered one, two, three, or four times per day. Doses may be administered until the desired therapeutic effect is achieved or indefinitely to maintain the desired therapeutic effect. Suitable dosing regimens for a compound of the invention depend on the pharmacokinetic properties of that compound, such as absorption, distribution, and half-life, which can be determined by the skilled artisan. In addition, suitable dosing regimens, including the duration such regimens are administered, for a compound of the invention depend on the condition being treated, the severity of the condition being treated, the age and physical condition of the patient being treated, the medical history of the patient being treated, the nature of concurrent therapy, the desired therapeutic effect, and like factors within the knowledge and expertise of the skilled artisan. It will be further understood by such skilled artisans that suitable dosing regimens may require adjustment given an individual patient's response to the dosing regimen or over time as individual patient needs change.

Typical daily dosages may vary depending upon the particular route of administration chosen. Typical daily dosages for oral administration, to a human weighing approximately 70 kg, would range from 50 mg to 3 g, suitably 100 mg to 2 g of a compound of the invention a day.

Additionally, the compounds of the invention may be administered as prodrugs. As used herein, a “prodrug” of a compound of the invention is a functional derivative of the compound which, upon administration to a patient, eventually liberates the compound of the invention in vivo. Administration of a compound of the invention as a prodrug may enable the skilled artisan to do one or more of the following: (a) modify the onset of the compound in vivo; (b) modify the duration of action of the compound in vivo; (C) modify the transportation or distribution of the compound in vivo; (d) modify the solubility of the compound in vivo; and (e) overcome a side effect or other difficulty encountered with the compound. Typical functional derivatives used to prepare prodrugs include modifications of the compound that are chemically or enzymatically cleaved in vivo. Such modifications, which include the preparation of phosphates, amides, esters, thioesters, carbonates, and carbamates, are well known to those skilled in the art.

The invention also provides a compound of the invention for use in medical therapy, particularly in bacterial infections. Thus, in a further aspect, the invention is directed to the use of a compound according to Formula I or a pharmaceutically-acceptable salt thereof in the preparation of a medicament for the treatment of bacterial infections.

The methods of treatment of the invention, specifically methods for the treatment infectious diseases including bacterial infections, comprise administering an effective amount of a compound according to Formula I, or a pharmaceutically acceptable salt thereof, to a patient in need thereof.

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide or a pharmaceutically acceptable salt thereof, to a human in need thereof.

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate to a human in need thereof.

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 to a human in need thereof.

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 to a human in need thereof.

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate).

In one aspect, the present invention provides for a method of treating a bacterial infection in humans comprising administration of a therapeutically effective amount of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide, or a pharmaceutically acceptable salt thereof, and at least on pharmaceutically acceptable excipient to a human in need thereof.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate and at least one pharmaceutically acceptable excipient to a human in need thereof.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 and at least one pharmaceutically acceptable excipient to a human in need thereof.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 and at least one pharmaceutically acceptable excipient to a human in need thereof.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesuphonate (or i.e., [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimesylate) and at least one pharmaceutically acceptable excipient to a human in need thereof.

In one aspect, the present invention provides for a method of treating a bacterial infection comprising administration of a therapeutically effective amount of a pharmaceutical composition comprising [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate and at least one pharmaceutically acceptable excipient to a human in need thereof.

For each of the aspects above, the bacterial infection recited above for each method, may be caused by Streptococcus, Staphylococcus, Moraxella, Haemophilus, Neisseria, Mycoplasma, Legionella, Chlamydia, Bacteroides, Clostridium, Fusobacterium, Propionibacterium, or Peptostreptococcus.

For each of the aspects above, the bacterial infection as recited for each method may be selected from an ear infection, sinusitis, upper respiratory tract infection, lower respiratory tract infection, genital infection, skin and soft tissue infection, bacterial endocarditis and the like.

### Biology and Biological Assays

As stated above, compounds according to Formula (I) or pharmaceutically acceptable salts thereof are useful in the inhibition of bacterial peptide deformylase (PDF) activity and in treatment methods for bacterial infections. The biological activity of the compounds according to Formula (I) or pharmaceutically acceptable salts thereof can be determined using suitable assays such as those measuring such inhibition and those evaluating the ability of the compounds to inhibit bacterial growth in vitro or in animal models of infection.

The biological activity of the compounds according to Formula (I) or pharmaceutically acceptable salts thereof can be determined using suitable assays such as those measuring inhibition of the enzymatic activity of PDF and those evaluating the ability of the compounds to inhibit bacterial growth in vitro or in animal models of infection.

Certain Examples of the invention possess greater in vitro antibacterial activity (MIC and/or MIC90) and/or better in vivo efficacy over the examples from WO 03/101442. These Examples include, but are not limited to, the following:
[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide.

### PDF IC50 Assay

Enzymatic activity of PDF was measured using a formate dehydrogenase (FDH)-coupled assay [Lazennec and Meinnel (1997) Anal. Biochem. 244, 180-182]. Once formate is released from methionine by PDF, it is oxidized by FDH thereby reducing one molecule of NAD to NADH and resulting in an increase in absorbance at 340 nm. Reactions were initiated by adding PDF to microtiter plates containing all other reaction components and were continuously monitored for 20 min at 25° C. The final reaction composition for the Staphylococcus aureus PDF (SaPDF) assay was 50 mM potassium phosphate, pH 7.6, 5 units/mL FDH, 7 mM NAD, 5% DMSO, 1 nM SaPDF, and 2.9 mM formyl-Met-Ala-Ser in 50 μL total volume. Serial dilutions of inhibitors were performed in DMSO. Reagents and assay format were identical for Haemophilus influenzae PDF except that formyl-Met-Ala-Ser was 6 mM final. In the Streptococcus pneumoniae PDF assay, reaction conditions were similar but contained 30 pM enzyme, 2 mM NAD and 4 mM formyl-Met-Ala-Ser. The varying formyl-Met-Ala-Ser concentrations reflect K_{M} values for substrate using the different PDF isozymes. IC₅₀s were determined by fitting to the equation: % Inhibition=100/1+(IC₅₀/[I])^{s}, where s is a slope factor, I is the inhibitor concentration and the IC₅₀ is the concentration of compound that causes 50% inhibition.

### Results

Examples 1-4 inhibit S. aureus, H. influenzae and S. pneumoniae PDF activities with IC50s<100 nM.

### Antimicrobial Activity Assay

Whole-cell antimicrobial activity was determined by broth microdilution using the Clinical and Laboratory Standards Institute (CLSI, formerly NCCLS) recommended methodology (NCCLS Document M7-A6, “Methods for Dilution Susceptibility Tests for Bacteria that Grow Aerobically—Approved Standard Sixth Edition”, 2003). Compounds were tested in serial two-fold dilutions ranging from 64 to 0.06 μg/mL. A panel of 12 strains was evaluated in the assay. This panel consisted of the following laboratory strains: Staphylococcus aureus Oxford, Staphylococcus aureus WCUH29, Enterococcus faecalis I, Enterococcus faecalis 7, Haemophilus influenzae Q1, Haemophilus influenzae NEMC1, Moraxella catarrhalis 1502, Streptococcus pneumoniae 1629, Streptococcus pneumoniae N1387, Streptococcus pneumoniae Ery2, Escherichia coli 7623 (AcrABEFD+) and Escherichia coli 120 (AcrAB−). The minimum inhibitory concentration (MIC) was determined as the lowest concentration of compound that inhibited visible growth. A mirror reader was used to assist in determining the MIC endpoint.

### Results

Each of the Examples 1-4 have a minimal inhibitory concentration (MIC)≦4 μg/mL against at least one of the organisms listed above. For at least one strain of every organism listed above, at least one example had an MIC≦4 μg/mL, with the exception of Enterococcus faecalis I, and Enterococcus faecalis 7, for which most examples had MICs≧16 μg/mL.

Antimicrobial Activity data (MIC's in μg/mL) for specific Examples is given in Table 2.

TABLE 2

| | Example 24* |
|---|---|
| Organism | μg/mL |
| S. aureus Oxford | 2 |
| S. aureus WCUH29 | 0.375 |
| E. faecalis I | 48 |
| E. faecalis 7 | 32 |
| H. influenzae Q1 | 0.5 |
| H. influenzae NEMC1 | 1 |
| M. catarrhalis 1502 | 0.375 |
| S. pneumoniae 1629 | 0.75 |
| S. pneumoniae N1387 | 0.5 |
| S. pneumoniae ERY2 | 0.375 |
| E. coli 7623 AcrABEFD+ | 16 |
| E. coli 120 AcrAB− | 0.1875 |
| Number of times entire panel of | 6 |
| 12 strains was run | |

*MIC data is expressed as the median of all results obtained

### Animal Models of Infection

All procedures were performed in accordance with protocols approved by the GSK Institutional Animal Care and Use Committee, and meet or exceed the standards of the American Association for the Accreditation of Laboratory Animal Care (AAALAC), the United States Department of Health and Human Services and all local and federal animal welfare laws.

Rat Respiratory Tract Infection (RTI) Model with H. influenzae or S. pneumoniae.

In this model, anesthetized rats (male Sprague Dawley [Cr1:CD (SD] 100 g) (Charles River) were infected by intrabronchial instillation of 2−3×10⁶ bacterial CFU/rat in 100 μL of agar directly into the lungs [G. Smith (1991) Lab Animals vol 25, 46-49]. Animals (n=6 per group) were dosed with different amounts of compound (2-fold dilution ranging from 37.5 to 300 mg/kg) by oral gavage twice daily for 4 days starting 1 h after infection. Control animals were dosed with diluent on the same schedule. The rats were euthanized 96 h post infection and the lungs removed aseptically and homogenized in 1 mL of sterile saline with a stomacher machine. Ten fold serial dilutions were done in sterile saline to enumerate viable bacteria numbers. This rat lung infection model has been shown to be able to predict human efficacy in community-acquired pneumonia (CAP) caused by S. pneumoniae [Hoover J. L., C. Mininger, R. Page, R. Straub, S. Rittenhouse, and D. Payne. (2007). Abstract A-17. Proceedings of the 47th ICAAC, Chicago, Ill.].

Murine Groin S. aureus Abscess Model of Skin and Soft Tissue Infection (SSTI).

In this model, anesthetized mice (male CD1, 20 g) (Charles River) were infected with S. aureus in semi-solid agar (1×10⁶ CFU/mouse) subcutaneously in the groin area (Jarvest, R. L., Berge, J. M., Berry, V., Boyd, H. F., Brown, M. J., Elder, J. S., Forrest, A. K., Fosberry, A. P., Gentry, D. R., Hibbs, M. J., Jaworski, D. D., O'Hanlon, P. J., Pope, A. J., Rittenhouse, S. Sheppard, R. J., Slater-Radosti, C. and Worby, A. (2002) J. Med. Chem., 45, 1959-1962). The animals (n=6 per group) were dosed with different amounts of compound (2-fold dilution ranging from 37.5 to 300 mg/kg) by oral gavage twice daily starting 1 h after infection. Control animals were dosed with diluent on the same schedule. Mice are euthanized 96 h post infection and the abscesses are aseptically removed and homogenized. Ten fold serial dilutions were done in sterile saline to enumerate viable bacteria numbers.

### Results

Some of the Examples described herein have demonstrated oral efficacy in one or more of the above animal models of infection, reducing the amount of bacteria recovered from lungs or abscesses, with respect to the untreated control animals, by ≧3 log₁₀ CFU/mL.

### EXAMPLES

The following examples illustrate the invention. These examples are not intended to limit the scope of the present invention, but rather to provide guidance to the skilled artisan to prepare and use the compounds, compositions, and methods of the present invention.

While particular embodiments of the present invention are described, the skilled artisan will appreciate that various changes and modifications can be made without departing from the spirit and scope of the invention.

As used herein the symbols and conventions used in these processes, schemes and examples are consistent with those used in the contemporary scientific literature, for example, the Journal of the American Chemical Society or the Journal of Biological Chemistry. Standard single-letter or three-letter abbreviations are generally used to designate amino acid residues, which are assumed to be in the L-configuration unless otherwise noted. Unless otherwise noted, all starting materials were obtained from commercial suppliers and used without further purification.

All references to ether are to diethyl ether; brine refers to a saturated aqueous solution of NaCl. Unless otherwise indicated, all temperatures are expressed in ° C. (degrees Centigrade). All reactions are conducted under an inert atmosphere at room temperature unless otherwise noted, and all solvents are highest available purity unless otherwise indicated.

¹H NMR (hereinafter also “NMR”) spectra were recorded on a Varian VXR-300, a Varian Unity-300, a Varian Unity-400 instrument, a Brucker AVANCE-400, a General Electric QE-300 or a Bruker AM 400 spectrometer. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), quint (quintet), m (multiplet), br (broad).

Mass spectra were run on an open access LC-MS system using electrospray ionization. LC conditions: 10% to 80% CH₃CN (0.018% TFA) in 3.0 min with a 1.25 min hold and 0.5 min re-equilibration; detection by MS, UV at 214 nm, and a light scattering detector (ELS). Column: 2.1×50 mm Zorbax SB-C8.

For preparative (prep) HPLC; ca. 100 mg of the final products were injected in 1000 μL of MeOH, DMSO, or DMF onto a SunFire Prep C18 OBD 5 um 30×75 mm column at 35 mL/min with a 10 min gradient from 5% CH₃CN to 95% CH₃CN in H₂O, followed by a 90% CH₃CN in H₂O hold for 1.9 min. Flash chromatography was run over Merck Silica gel 60 (230-400 mesh), or using a Teledyne Isco Combiflash Companion with normal phase, disposable Redi-Sep flash columns.

XRPD spectrum were recorded using a PANalytical X'Pert Pro MPD-XRD, PW3040.

ATR-IR spectrum were recorded using a Thermo Electron Nexus 470 FTIR with Diamond ATR accessory.

### INTERMEDIATES

### Intermediate A

### (2R)-3-Cyclopentyl-2-({formyl[(phenylmethyl)oxy]amino}methyl)propanoic acid

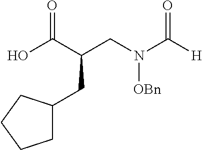 [SMILES: O=CN(C[C@@H](CC1CCCC1)C(=O)O)OCc1ccccc1]

### Part A

### (4S)-Benzyl-3-(3-cyclopentylpropanoyl)oxazolidin-2-one

To a solution of (S)-(−)-4-benzyl-2-oxazolidinone (25 g, 141 mmol) in THF (350 mL) at −78° C. was added dropwise n-BuLi (56.4 mL, 2.5M solution in hexane, 141 mmol). After stirring for 60 min at the same temperature, the reaction mixture was then treated with 3-cyclopentylpropionyl chloride (21.6 mL, 141 mmol) over 0.25 h. The reaction mixture was allowed to warm to room temperature and stirred overnight. The reaction was quenched with saturated aqueous NH₄Cl solution (320 mL). The aqueous layer was extracted with EtOAc (3×200 mL). The combined organic layers were dried (MgSO₄) and evaporated to yield (4S)-benzyl-3-(3-cyclopentylpropanoyl)oxazolidin-2-one as a white solid (42.4 g, 100%). LCMS: (M+H)⁺: 302.3.

### Part B

### (4S)-3-(2R)-3-Cyclopentyl-2-{[(phenylmethyl)oxy]methyl}propanoyl)-4-(phenylmethyl)-1,3-oxazolidin-2-one

To a solution of (4S)-benzyl-3-(3-cyclopentylpropanoyl)oxazolidin-2-one (42.4 g, 141 mmol) in dichloromethane (500 mL) at 0° C. under nitrogen was added titanium (IV) chloride (1 M in DCM, 155 mL, 155 mmol) in a slow steady stream. After 5 min, diisopropylethylamine (27 mL, 155 mmol) was added dropwise. After stirring at 0° C. for 1 h, benzyloxymethylchloride (TCI-America) (39 mL, 280 mmol) was added in a slow steady stream to the resulting titanium enolate, and the mixture was maintained at 0° C. for 3.5 h. The reaction mixture was then quenched with water (400 mL). The aqueous layer was extracted with dichloromethane (150 mL×2). The organic extracts were washed with saturated NaHCO₃, were dried (MgSO₄) and were evaporated. The residue was washed with ether (2×), and then was triturated with hexanes/ether to provide 2 crops of (4S)-3-((2R)-3-cyclopentyl-2-{[(phenylmethyl)oxy]methyl}propanoyl)-4-(phenylmethyl)-1,3-oxazolidin-2-one as a pale yellow solid (42.7 g, 72%). LCMS: (M+H)⁺: 422.2.

### Part C

### (4S)-3-[(2R)-3-Cyclopentyl-2-(hydroxymethyl)propanoyl]-4-(phenylmethyl)-1,3-oxazolidin-2-one

A solution of (4S)-3-((2R)-3-cyclopentyl-2-{[(phenylmethyl)oxy]methyl}propanoyl)-4-(phenylmethyl)-1,3-oxazolidin-2-one (42.7 g, 0.1 mol) in ethanol (800 mL) and DMF (180 mL) was subjected to catalytic hydrogenation using 10% Pd/C (4 g) and a balloon of hydrogen. The reaction was 50% complete by LCMS after 24 h. The reaction was purged with nitrogen and a fresh balloon of hydrogen was introduced. After an additional 60 h, the reaction was again purged with nitrogen, was filtered, and the filtrate solvents were removed to provide (4S)-3-[(2R)-3-cyclopentyl-2-(hydroxymethyl)propanoyl]-4-(phenylmethyl)-1,3-oxazolidin-2-one (33.1 g, 100%). LCMS: (M+H)⁺: 332.3.

### Part D

### (2R)-3-Cyclopentyl-2-(hydroxymethyl)propanoic acid

(4S)-3-[(2R)-3-Cyclopentyl-2-(hydroxymethyl)propanoyl]-4-(phenylmethyl)-1,3-oxazolidin-2-one (33.1 g, 0.1 mol) was stirred in a mixture of THF (330 mL) and water (55 mL) and cooled to 0° C. 30% Hydrogen peroxide (96 mL, 1 mol) was added, followed by lithium hydroxide monohydrate (8.4 g, 0.2 mol). The reaction was warmed to room temperature, and then stirred overnight. The THF was removed by rotary evaporation. The aqueous residue was washed with dichloromethane (3×100 mL), was acidified with 6N HCl, and was extracted with ethyl acetate (4×100 mL). The organic extracts were dried (MgSO₄) and were evaporated to provide (2R)-3-cyclopentyl-2-(hydroxymethyl)propanoic acid as a clear, colorless oil (18.5 g, >100%). LCMS: (M+H)⁺: not detected. ¹H NMR (400 MHz, CDCl₃): δ 7.3 (br s, 1H), 3.79 (d, J=5.83 Hz, 2H), 2.64-2.71 (m, 1H), 1.45-1.87 (m, 9H), 1.05-0.14 (m, 2H).

### Part E

### (2R)-3-Cyclopentyl-2-(hydroxymethyl)-N-[(phenylmethyl)oxy]propanamide

To a mixture of (2R)-3-cyclopentyl-2-(hydroxymethyl)propanoic acid (18.3 g, 106 mmol), O-benzyl hydroxyamine hydrochloride (18.62 g, 117 mmol) and 4-(dimethylamino)pyridine (28.5 g, 233 mmol) in dichloromethane (110 mL) at 0° C. was added 1-[3-(dimethylamino)propyl]-3-ethylcarbodiimide hydrochloride (22.3 g, 117 mmol). The mixture was maintained at 0° C. for 3 h. After this time, 500 mL of 1N cold, aqueous HCl solution was added, and the mixture was stirred for another 30 min. The resulting white solid precipitate was collected by filtration. The precipitate was washed with 1N HCl, with water, and with cold DCM. Drying overnight in a vacuum dessicator provided (2R)-3-cyclopentyl-2-(hydroxymethyl)-N-[(phenylmethyl)oxy]propanamide (19.1 g, 65%). LCMS: (M+H)⁺: 278.1.

### Part F

### (3R)-3-(Cyclopentylmethyl)-1-[(phenylmethyl)oxy]-2-azetidinone

To a mixture of (2R)-3-cyclopentyl-2-(hydroxymethyl)-N-[(phenylmethyl)oxy]propanamide (22.5 g, 81 mmol) and triphenylphosphine (22.5 g, 97 mmol) in THF (800 mL) at 0° C. was added dropwise diisopropyl azodicarboxylate (18.9 mL, 97 mmol). The reaction mixture was maintained at 0° C. for 45 min and was then evaporated. Purification by chromatography on silica gel using an eluting system of hexane/EtOAc (95:5) provided (3R)-3-(cyclopentylmethyl)-1-[(phenylmethyl)oxy]-2-azetidinone (16.9 g, 81%). LCMS: (M+H)⁺: 260.1.

### Part G

### (2R)-3-Cyclopentyl-2-({[(phenylmethyl)oxy]amino}methyl)propanoic acid

A mixture of (3R)-3-(cyclopentylmethyl)-1-[(phenylmethyl)oxy]-2-azetidinone (20 g, 77.1 mmol) and LiOH.H₂O (32.4 g, 0.77 mol) in THF/water (500 mL/170 mL) was stirred at room temperature for 36 h. To the reaction mixture was added 6M HCl (130 mL), and then 1 N NaOH was added until a neutral pH was obtained. The layers were separated, and the aqueous layer was extracted once with EtOAc. The combined organics were dried (MgSO₄) and concentrated to provide (2R)-3-cyclopentyl-2-({[(phenylmethyl)oxy]amino}methyl)propanoic acid (22.85 g, >100%) as a clear, colorless oil. LCMS: (M+H)⁺: 277.9.

### Part H

### (2R)-3-Cyclopentyl-2-({formyl[(phenylmethyl)oxy]amino}methyl)propanoic acid

Under a nitrogen atmosphere, formic acid (192 mL, 5 mol) was dissolved in CH₂Cl₂ (450 mL) and cooled to 0° C. Acetic anhydride (73 mL, 0.77 mol) was then added, and the reaction mixture was stirred for 45 min. After this time, a solution of (2R)-3-cyclopentyl-2-({[(phenylmethyl)oxy]amino}methyl)propanoic acid (22.85 g crude material, assumed 77.1 mmol) in CH₂Cl₂ (450 mL) was added, and the resulting mixture was stirred for 1.5 h at 0° C. The volatiles were then removed, the crude residue was dissolved in EtOAc (500 mL), and the mixture was washed with brine (4×100 mL). The organics were dried (MgSO₄) and concentrated to provide (2R)-3-cyclopentyl-2-({formyl[(phenylmethyl)oxy]amino}methyl)propanoic acid (23.5 g, 100%) as a thick syrup. LCMS: (M+H)⁺: 306.1.

The (2R)-3-cyclopentyl-2-({formyl[(phenylmethyl)oxy]amino}methyl)propanoic acid, diisopropylethylamine salt, isopropanol solvate can be prepared in the following manner:

To a solution of (2R)-3-cyclopentyl-2-({formyl[(phenylmethyl)oxy]amino}methyl)propanoic acid (25.9 h, 85 mmol) in diethyl ether (85 mL) was added diisopropylethylamine (19.7 mL, 113 mmol), and the mixture was stirred at room temperature for approximately 3 h. The reaction mixture was then diluted with additional diethyl ether (85 mL) and water (400 mL). The layers were separated, and the organic layer was extracted two more times with a water/brine mixture (250 mL water with 30 mL brine added and 200 mL water with 30 mL brine added). The combined aqueous layers were then extracted with 40% isopropanol in chloroform (3×300 mL). The combined isopropanol/chloroform layers were dried (Na₂SO₄), filtered and evaporated to provide the 2(R)-3-cyclopentyl-2-({formyl[(phenylmethyl)oxy]amino}methyl)propanoic acid, diisopropylethylamine salt, isopropanol solvate (30.29 g) as a clear beige oil. LCMS: (M+H)⁺: 306.2.

### Intermediate B

### (2R)-3-Cyclopentyl-2-{[formyl(tetrahydro-2H-pyran-2-yloxy)amino]methyl}propanoic acid

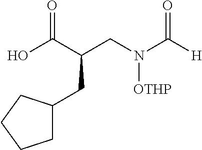

### Part A

### (3R)-3-(Cyclopentylmethyl)-1-(tetrahydro-2H-pyran-2-yloxy)-2-azetidinone

(3R)-3-(Cyclopentylmethyl)-1-[(phenylmethyl)oxy]-2-azetidinone (100 g, 386 mmol) was dissolved in ethanol (1.2 L), and the solution was degassed. Pd on C (10%, dry, 8 g) was added and the suspension was purged with hydrogen and stirred under a hydrogen atmosphere (balloon) until the reaction was complete by LC-MS (approximately 6 h). The suspension was then sparged with nitrogen, filtered through Celite, and evaporated to dryness. The resulting solid was redissolved in CH₂Cl₂ (1 L) and dihydropyran (70 mL, 767 mmol) was added, followed by pyridinium p-toluenesulfonate (PPTS, 5%, 4.85 g). The reaction mixture was stirred 3 days at room temperature, then concentrated and chromatographed on silica gel using 10-20% ethyl acetate in hexanes to provide (3R)-3-(cyclopentylmethyl)-1-(tetrahydro-2H-pyran-2-yloxy)-2-azetidinone as a colorless liquid (100%).

### Part B

### (2R)-3-Cyclopentyl-2-{[(tetrahydro-2H-pyran-2-yloxy)amino]methyl}propanoic acid

(3R)-3-(Cyclopentylmethyl)-1-(tetrahydro-2H-pyran-2-yloxy)-2-azetidinone (68 g, 268 mmol) was dissolved in THF (1 L) and placed in a 3-necked 3 L round bottomed flask that had been fitted with an internal thermocouple, reflux condenser, and a mechanical stirrer. A solution of lithium hydroxide monohydrate (56.3 g, 1.34 mol) in 400 mL H₂O was prepared and added dropwise via the addition funnel, with vigorous stirring. The reaction mixture was stirred at room temperature for 36 h before being diluted with H₂O (350 mL) and washed with hexanes (300 mL). The organic layer was extracted with H₂O (100 mL) and the combined aqueous layers were cooled to 0° C. and carefully acidified with 2M citric acid (˜525 mL) drop wise over the course of 90 min, keeping the internal temperature below 10° C. The acidified material was extracted with ethyl acetate (3×250 mL) and the combined organic layers were washed with water (2×), dried over MgSO₄, filtered, and evaporated. Benzene (500 mL) was added and evaporated, and the residue was dried in vacuo to obtain (2R)-3-cyclopentyl-2-{[(tetrahydro-2H-pyran-2-yloxy)amino]methyl}propanoic acid (70.9 g, 98%) as a colorless liquid.

### Part C

### (2R)-3-Cyclopentyl-2-{[formyl(tetrahydro-2H-pyran-2-yloxy)amino]methyl}propanoic acid

To a solution of (2R)-3-cyclopentyl-2-{[(tetrahydro-2H-pyran-2-yloxy)amino]methyl}propanoic acid (97.05 g, 358 mmol) in acetone (1.1 L) at room temperature was added 5-methyl-2-thioxo-[1,3,4]thiadiazole-3-carbaldehyde (57.3 g, 358 mmol) (Tetrahedron Lett. 1985, 26, 3703-3706). When the reaction was deemed complete, the acetone was removed in vacuo. The residue was suspended in a mixture of hexanes (320 mL) and methyl-t-butyl ether (180 mL), then sonicated. After 10 min, the white solid (presumably 5-methyl-3H-[1,3,4]thiadiazole-2-thione) was filtered off, and the filtrate was evaporated in vacuo to give (2R)-3-cyclopentyl-2-{[formyl(tetrahydro-2H-pyran-2-yloxy)amino]methyl}propanoic acid as a pale yellow gum (124 g, >100%). NMR shows the product contains a small amount of MTBE and 5-methyl-3H-[1,3,4]thiadiazole-2-thione.

(2R)-3-Cyclopentyl-2-{[formyl(tetrahydro-2H-pyran-2-yloxy)amino]methyl}propanoic acid can also be prepared according to literature procedures [Bracken, Bushell, Dean, Francavilla, Jain, Lee, Seepersaud, Shu, Sundram, Yuan; PCT Int. Appl. (2006), WO 2006127576 A2].

The (2R)-3-cyclopentyl-2-{[formyl(tetrahydro-2H-pyran-2-yloxy)amino]methyl}propanoic acid, diisopropylethylamine salt can be prepared in the following manner:

A solution of (2R)-3-cyclopentyl-2-{[(tetrahydro-2H-pyran-2-yloxy)amino]methyl}propanoic acid (39.45 g, 145 mmol) in methyl formate (300 mL) and diisopropylethylamine (27.9 mL, 160 mmol) was placed in a sealed tube and heated to 50° C. for 4 days. After cooling to room temperature, the methyl formate was removed in vacuo, and the remaining residue was dissolved in diethyl ether. The ether solution was extracted with water, and the layers were separated. The aqueous layer was then back-extracted with a solution of 40% isopropanol in chloroform (2×). The combined isopropanol/chloroform layers were then concentrated in vacuo to provide (2R)-3-cyclopentyl-2-{[formyl(tetrahydro-2H-pyran-2-yloxy)amino]methyl}propanoic acid as the diisopropylethyl amine salt (28 g, containing a residual amount of chloroform and isopropanol).

### Intermediate C

### 4,6-Dichloro-5-fluoro-2-methylpyrimidine

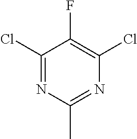

### Part A

### 5-Fluoro-4,6-dihydroxy-2-methylpyrimidine

A solution of 200 mL of 25% wt sodium methoxide in methanol (0.84 mol) was diluted with an additional 200 mL of methanol. Acetamidine-HCl (40 g, 0.42 mol) was added to the sodium methoxide solution (white precipitate formed), followed by addition of dimethyl fluoromalonate (70 g, 0.46 mol). The contents were stirred at room temperature overnight, then concentrated in vacuo to dryness. The resulting residue was redissolved in hot water (300 mL). After cooling the aqueous solution to room temperature, concentrated HCl was added slowly until crystal formation (fine white prisms) took place at about pH 5. Concentrated HCl was added dropwise until pH 3, and then the contents were filtered. The isolated crystals were rinsed with 1M HCl and dried under vacuum to provide 5-fluoro-4,6-dihydroxy-2-methylpyrimidine (65.5 g, >100%). LCMS: (M+H)⁺: 145.

### Part B

### 4,6-Dichloro-5-fluoro-2-methylpyrimidine

5-Fluoro-4,6-dihydroxy-2-methylpyrimidine (assumed 60 g, 0.42 mol) was treated with 300 mL of POCl₃ at 120° C. for 3 h. The reaction mixture was then cooled to room temperature and concentrated in vacuo until the rate of solvent removal slowed to a drop rate of less than 1 drop/second. The product is somewhat volatile and excessive concentration in vacuo will reduce the yield. The crude residue was poured over crushed ice, and the resulting slurry was stirred for 1 h, during which time the solution came to room temperature. A yellow solid formed which was filtered off, washed with water, and air dried briefly until free flowing. This solid was collected and placed in a dessicator over P₂O₅ until dry, providing pure 4,6-dichloro-5-fluoro-2-methylpyrimidine (59 g, 79%). LCMS: (M+H)⁺: 181/183.

### Intermediate D

### 4,6-Dichloro-2-ethyl-5-fluoropyrimidine

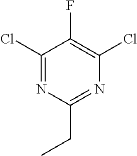

### Part A

### 2-Ethyl-5-fluoro-6-hydroxy-4(1H)-pyrimidinone

Propionamide hydrochloride salt (30.0 g, 276.3 mmol) and dimethyl fluoromalonate (41.4 g, 276.3 mmol) in anhydrous methanol (400 mL) were treated with solid NaOMe (45 g, 829 mmol) portion-wise at room temperature. After the addition, the white suspension was heated to 85° C. and stirred for 2 h. The solvent was then evaporated to dryness. To the residue was added 70 mL of 6 N HCl solution with vigorous stirring. The suspension was stirred for 10 min until the residue was fully neutralized. The white precipitate was collected by filtration and dried over vacuum to give 2-ethyl-5-fluoro-6-hydroxy-4(1H)-pyrimidinone as a white solid. LCMS: (M+H)⁺: 159.0; (M+Na)+:181.1. In some cases, this product may contain co-precipitated NaCl, causing the yield to exceed the theoretical value. In such cases, this product was carried forward through the next step with the NaCl present.

### Part B

### 4,6-Dichloro-2-ethyl-5-fluoropyrimidine

2-Ethyl-5-fluoro-6-hydroxy-4(1H)-pyrimidinone (20 g, 126.6 mmol) in POCl₃ (58 mL, 633 mmol) was heated at 125° C. (oil bath) for 2 h. An additional 68 mL of fresh POCl₃ was added to the hot solution. The resulting solution was heated for an additional 2 h until all the starting material was consumed. The excess POCl₃ was distilled (62° C.-68° C.) in vacuo to give a light brown residue. After being cooled to room temperature, the residue was diluted with 50 mL of CH₂Cl₂, then poured into ice water (200 mL). To this mixture was added 200 mL of CH₂Cl₂ and the subsequent mixture was stirred for 10 min. After separation of the two layers, the aqueous layer was further extracted with 100 mL of CH₂Cl₂. The combined organic layers were dried over Na₂SO₄, then filtered through a short silica gel pad, which was then washed with 150 mL of 1% MeOH in CH₂Cl₂. Evaporation of the solvent provided 4,6-dichloro-2-ethyl-5-fluoropyrimidine (21 g, 85%) as a light yellow liquid. LCMS: (M+H)⁺: not detected.

### Intermediate E

### [(2R)-3-{2-[6-Chloro-5-fluoro-2-(methylthio)-4-pyrimidinyl]hydrazino}-2-(cyclopentyl methyl)-3-oxopropyl](tetrahydro-2H-pyran-2-yloxy)formamide

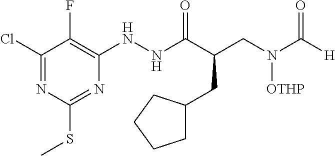

### Part A

### 5-Fluoro-6-hydroxy-2-methylthio-4(1H)-pyrimidinone

To a stirred solution of 2-methyl-2-thiopseudourea sulfate (41.7 g, 0.15 mol) and dimethyl fluoromalonate (45 g, 0.30 mol) in MeOH (600 mL) at 0° C. (ice bath) was added NaOMe (48.6 g, 0.90 mol) in portions. After the addition was complete, the ice bath was withdrawn and the reaction mixture was stirred at room temperature overnight. LCMS showed the formation of the desired pyrimidinone product. The reaction mixture was concentrated to near dryness under vacuum, diluted with water (50 mL), and acidified with 6N HCl (˜150 mL) to ˜pH 2 to precipitate the product. After filtration, the solid was washed with 1N HCl (2×10 mL) and dried under vacuum to afford 5-fluoro-6-hydroxy-2-methylthio-4(1H)-pyrimidinone (35.7 g, 68%) as a white solid. LCMS: (M+H)⁺: 177.3.

### Part B

### 4,6-Dichloro-5-fluoro-2-(methylthio)pyrimidine

A mixture of 5-fluoro-6-hydroxy-2-(methylthio)-4(1H)-pyrimidinone (35.7 g, 0.20 mol) in POCl₃ (150 mL) was heated at 115° C. for 3 h. After cooling to room temperature, the reaction mixture was slowly poured into an ice-water mixture (1500 mL) and stirred for 20 min. The product was extracted into ethyl acetate (3×800 mL), and the combined organic extracts were washed with water (2×1000 mL), brine (1000 mL), and dried (Na₂SO₄). Evaporation of the solvent provided 4,6-dichloro-5-fluoro-2-(methylthio)pyrimidine as a pale yellow solid (37.8 g, 89%). LCMS: (M+H)⁺: not detected.

### Part C

### 4-Chloro-5-fluoro-6-hydrazino-2-(methylthio)pyrimidine

4,6-Dichloro-5-fluoro-2-(methylthio)pyrimidine (16.8 g, 78.85 mmol) and triethylamine (16.49 mL, 118.3 mmol) were dissolved in DMSO (200 mL) and stirred. The mixture was cooled to ˜5° C. with an ice water bath. To this solution was slowly added hydrazine monohydrate (4.59 mL, 94.62 mmol). After the addition was complete, the reaction mixture was warmed up to RT and stirring was continued for 1 h. The reaction mixture was diluted with water (500 mL), and the aqueous solution was extracted with CH₂Cl₂ (3×300 mL). The combined organic solution was washed with water (3×250 mL) and brine (250 mL), then dried (Na₂SO₄) and concentrated in vacuo to afford 4-chloro-5-fluoro-6-hydrazino-2-(methylthio)pyrimidine as a red foamy solid (9.70 g, 59%). LCMS: (M+H)⁺: 208.9.

### Part D

### [(2R)-3-{2-[6-Chloro-5-fluoro-2-(methylthio)-4-pyrimidinyl]hydrazino}-2-(cyclopentylmethyl)-3-oxopropyl](tetrahydro-2H-pyran-2-yloxy)formamide

A mixture of 4-chloro-5-fluoro-6-hydrazino-2-(methylthio)pyrimidine (9.70 g, 46.5 mmol), (2R)-3-cyclopentyl-2-{[formyl(tetrahydro-2H-pyran-2-yloxy)amino]methyl}propanoic acid (19.9 g, 46.5 mmol), HOAt (6.96 g, 51.2 mmol), EDCI (9.82 g, 51.2 mmol) and N-methyl morpholine (25.6 mL, 232.5 mmol) in DMF (300 mL) was stirred at room temperature overnight. The reaction mixture was diluted with ethyl acetate/hexanes (3:2, 1 L) and washed with water (3×500 mL), and the organics were dried (Na₂SO₄) and concentrated in vacuo. The residue was purified by Gilson RP-HPLC (35-95% acetonitrile/water, 8 min gradient time) to afford [(2R)-3-{2-[6-chloro-5-fluoro-2-(methylthio)-4-pyrimidinyl]hydrazino}-2-(cyclopentylmethyl)-3-oxopropyl](tetrahydro-2H-pyran-2-yloxy)formamide as a red glass (12.89 g, 56.0%). LCMS: (M+H)⁺: 490.4.

### Intermediate F

### 2,4,6-Trichloro-5-fluoropyrimidine

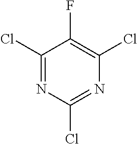

### Part A

### 5-Fluoro-6-hydroxy-2,4(1H,3H)-pyrimidinedione

A mechanically stirred solution of urea (60.06 g, 1 mol) and dimethyl fluoromalonate (150.11 g, 1 mol) in methanol (1 L) was treated with 25 wt % NaOMe in methanol (˜4.6 M, 435 mL, 2 mol). The mixture was refluxed for 3 h and then allowed to cool to room temperature. The mixture was filtered, the wet cake was dissolved in warm water (˜1.2 L), and the resulting aqueous solution was acidified with concentrated aqueous HCl (˜160 mL) to pH=2 while stirring over 1 h. The mixture was allowed to cool to room temperature, and the product was filtered and washed thoroughly with water, then dried under vacuum to give 5-fluoro-6-hydroxy-2,4(1H,3H)-pyrimidinedione (80 g, 55%) as a white solid. LCMS: (M+H)⁺: 147.0.

### Part B

### 2,4,6-Trichloro-5-fluoropyrimidine

Finely powdered 5-fluoro-6-hydroxy-2,4(1H,3H)-pyrimidinedione (74 g, 0.507 mol) was added portionwise over 30 min to POCl₃ (232 mL, 2.5 mol) with stirring (exothermic). Upon complete addition, the mixture was held at 60° C. while N,N,-dimethylaniline (65 mL) was added dropwise by syringe. After addition, the mixture was heated to 100-110° C. (internal) until the reaction was judged complete, usually in 4-8 h. The mixture was cooled and the bulk of the remaining POCl₃ was removed by careful vacuum distillation at 80-90° C. (some product can be detected in the POCl₃ distillate). The remaining residue was poured onto ice (˜1 L) and stirred for 30 min, then extracted with ether (1×400 mL, 2×150 mL). The combined extracts were washed with water and brine, then dried (MgSO₄). Filtration and atmospheric distillation of the ether provided the crude product, which was distilled under reduced pressure to provide the product (28.8 g, 28%) as a low melting white crystalline solid (b.p. 80-85° C., 12 mm). LCMS: (M+H)⁺: not detected.

### Intermediate G

### Tris(1,1-dimethylethyl)-2-(2,6-dichloro-5-fluoro-4-pyrimidinyl)-1,1,2-hydrazine tricarboxylate

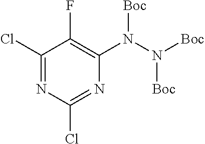 [SMILES: CC(C)(C)OC(=O)N(C(=O)OC(C)(C)C)N(C(=O)OC(C)(C)C)c1nc(Cl)nc(Cl)c1F]

### Part A

### 1,1-Dimethylethyl 2-(2,6-dichloro-5-fluoro-4-pyrimidinyl)hydrazinecarboxylate

2,4,6-Trichloro-5-fluoropyrimidine (20.92 g, 104.1 mmol) was dissolved in THF (300 mL) at room temperature and stirred. To this stirring solution was added t-butyl carbazate (13.74 g, 104.1 mmol), followed by diisopropylethylamine (19.0 mL, 109.3 mmol). The reaction mixture turned light yellow, and after several minutes a precipitate formed. The reaction appeared complete after 1.5 h, as monitored by TLC (10% EtOAc/Hex). The reaction mixture was concentrated in vacuo to remove most of the THF, and the residue was dissolved in CH₂Cl₂ (˜400 mL). The solution was washed with ˜400 mL of sat. aq. NH₄Cl. The organics were dried and concentrated to give a pale yellow solid (31.37 g). LCMS: (M+H+2Na-Boc)⁺: 241.

### Part B

### Tris(1,1-dimethylethyl)-2-(2,6-dichloro-5-fluoro-4-pyrimidinyl)-1,1,2-hydrazine tricarboxylate

1,1-Dimethylethyl 2-(2,6-dichloro-5-fluoro-4-pyrimidinyl)hydrazinecarboxylate (31.37 g, 104.1 mmol assumed) was suspended in CH₂Cl₂ (400 mL). Di-t-butyl dicarbonate (44.75 g, 205.0 mmol) was added to the solution, followed by diisopropylethylamine (36.3 mL, 208.2 mmol). When almost everything was dissolved, DMAP (1.27 g, 10.4 mmol) was added slowly. The reaction mixture turned reddish, and after ˜5 min, mild bubbling was observed. After 45 min, the reaction appeared complete by LCMS, and the mixture had turned light orange. The reaction mixture was washed with ˜300 mL sat. NH₄Cl, and the organics were set aside. A slurry was prepared with ˜1800 mL Florsil in CH₂Cl₂, which was poured onto a large fritted funnel. The entire organic solution was then poured through the Florsil pad, washing with 2 L of CH₂Cl₂. A red band was left behind on the Florsil, and TLC showed that the product had finished eluting from the pad. The filtrate was concentrated to a foamy colorless oil, which crystallized overnight in the refrigerator (37.87 g, 73% from 2,4,6-trichloro-5-fluoropyrimidine). LCMS: (M+3H+2Na-3Boc)⁺: 241.

### Intermediate H

### 4,6-Dichloro-5-fluoro-2-(fluoromethyl)pyrimidine

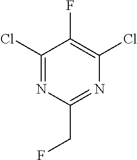 [SMILES: FCc1nc(Cl)c(F)c(Cl)n1]

### Part A

### 5-Fluoro-2-(fluoromethyl)-6-hydroxy-4(1H)-pyrimidinone

2-Fluoro-acetamidine hydrochloride salt (11.2 g, 100 mmol) and dimethyl fluoromalonate (15 g, 100 mmol) in anhydrous methanol (300 mL) were treated with solid NaOMe (16.2 g, 300 mmol) and heated to 50° C. with stirring. When LCMS showed formation of the desired product, the solvent was evaporated to dryness, and the residue was neutralized with concentrated HCl (20 mL). The white precipitate was collected by filtration to give 5-fluoro-2-(fluoromethyl)-6-hydroxy-4(1H)-pyrimidinone (100% yield). LCMS: (M+H)⁺: 163.1.

### Part B

### 4,6-Dichloro-5-fluoro-2-(fluoromethyl)pyrimidine

5-Fluoro-2-(fluoromethyl)-6-hydroxy-4(1H)-pyrimidinone (6 g, 37 mmol) was suspended in POCl₃ (20 mL, 222 mmol) and stirred at 120° C. for 2 h. After evaporation of the excess POCl₃, the residue was poured onto ice and the resulting mixture was extracted with CH₂Cl₂. The CH₂Cl₂ solution was passed through a silica gel pad, and the resulting filtrate was concentrated to provide the pure 4,6-dichloro-5-fluoro-2-(fluoromethyl)pyrimidine (6 g, 81%) as a colorless liquid. LCMS: (M+H)⁺: not detected.

### Intermediate I

### 4,6-Dichloro-2-(difluoromethyl)-5-fluoropyrimidine

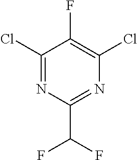 [SMILES: Fc1c(Cl)nc(C(F)F)nc1Cl]

### Part A

### 2,2-Difluoroethanimidamide.HCl

To a stirred suspension of ammonium chloride (5.1 g, 95 mmol) in toluene (150 mL) at 0° C. was added trimethyl aluminum (46 mL, 2M, 92 mmol), stirring until effervescence ceased. Methyl difluoroacetate (2.38 mL, 27 mmol) was added, and the resulting mixture was stirred overnight at 80° C. Upon cooling to 0° C., methanol was added slowly and the resulting solution was stirred for 90 minutes at reduced temperature, causing a solid to form. This was removed by filtration through Celite, and the filtrate was evaporated to yield 2,2-difluoroethanimidamide.HCl (1.7 g, 48 5%) as a yellow tinged solid.

### Part B

### 2-(Difluoromethyl)-5-fluoro-6-hydroxy-4(1H)-pyrimidinone

Sodium metal (0.91 g, 40 mmol) was dissolved in MeOH (100 mL) to form sodium methoxide. 2,2-Difluoroethanimidamide.HCl (1.73 g, 13 mmol) was added followed by dimethyl fluoropropanedioate (2.0 g, 13 mmol). The resulting solution was stirred at 80° C. for 3 hours, then cooled to room temperature. Aqueous HCl (6 mL, 6M, 36 mmol) was added and the resulting mixture was concentrated in vacuo. The remaining solid was washed with cold water and filtered yielding 2-(difluoromethyl)-5-fluoro-6-hydroxy-4(1H)-pyrimidinone (1.43 g, 61%)

### Part C

### 4,6-Dichloro-2-(difluoromethyl)-5-fluoropyrimidine

A mixture of 2-(difluoromethyl)-5-fluoro-6-hydroxy-4(1H)-pyrimidinone (1.43 g, 8.0 mmol) and POCl₃ (6 mL) was heated at 110° C. for 2.5 hours. After cooling to room temperature, the reaction mixture was poured over ice and stirred for 30 min. The product was extracted into DCM and the combined organics were washed once with aqueous saturated sodium bicarbonate. The combined organics were dried over sodium sulfate and concentrated in vacuo. This yielded 4,6-dichloro-2-(difluoromethyl)-5-fluoropyrimidine (460 mg, 27%) as a yellow oil.

### COMPOUND EXAMPLES

### Example 1

### [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide

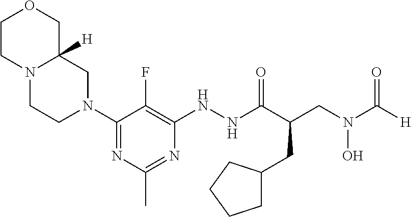 [SMILES: Cc1nc(NNC(=O)[C@H](CC2CCCC2)CN(O)C=O)c(F)c(N2CCN3CCOC[C@@H]3C2)n1]

### Part A

### N-(Phenylmethyl)-D-serine

As per the procedures of WO2005058245, a mixture of D-serine methyl ester hydrochloride (98.84 g, 635.3 mmol) in MeOH (280 mL) was cooled to 10° C. To the mixture was slowly added triethylamine (88.5 mL, 635.0 mmol). The mixture was warmed to room temperature and the resulting solution was cooled to 10° C. To the solution was added benzaldehyde (64 mL, 630.2 mmol), and the solution was stirred for 30 min. To the solution was added sodium borohydride (24.03 g, 635.2 mmol) portionwise over 30 min, and the mixture was stirred for a further 30 min. In a separate flask, MeOH (114 mL) was added to water (170 mL), and to this solution was added a solution of NaOH (77.25 g, 1931 mmol) in water (155 mL). The solution was cooled to 15° C., and the reductive amination mixture was slowly added to the NaOH-water-MeOH solution over 15 min. The solution was stirred and warmed to room temperature over 30 min, and water (170 mL) was added, followed by sufficient 6 N aqueous HCl to adjust the pH to 9.5. The solution was washed with EtOAc (2×60 mL), and sufficient 6 N aqueous HCl was added to adjust the pH to 6.5. The mixture was cooled to 0° C. and held overnight. The resulting solid was collected by vacuum filtration and washed with water (2×200 mL) followed by heptane (2×200 mL). The white solid was dried at 40° C. under high vacuum for 3 days to afford N-(phenylmethyl)-D-serine (79.51 g, 64%). LCMS: (M+H)⁺: 196.1.

### Part B

### (3R)-5-Oxo-4-(phenylmethyl)-3-morpholinecarboxylic acid

As per the procedures of WO2005058245, a solution of N-(phenylmethyl)-D-serine (79.51 g, 407.3 mmol) in THF (485 mL) was cooled to 0° C., and a precooled 0° C. solution of K₂CO₃ (168.87 g, 1222 mmol) in water (485 mL) was added. To the well-stirred mixture was added chloroacetyl chloride (45.4 mL, 570.0 mmol) slowly while keeping the internal temperature below 5° C. The mixture was vigorously stirred at 0° C. for 30 min, and then an additional portion of chloroacetyl chloride (4.54 mL, 57.0 mmol) was slowly added. The mixture was stirred for an additional 30 min at 0° C. To the mixture was added a sufficient quantity of precooled 0° C. aqueous NaOH (50% w/w) to adjust the pH>13.5 while keeping the internal temperature between 5° C. and 10° C. The mixture was stirred at 0° C. for 2 h, and then warmed to 20° C. The mixture was washed with heptane (165 mL) followed by a second portion of fresh heptane (240 mL). The aqueous phase was cooled to 0° C., and adjusted to pH<2 with concentrated aqueous HCl while keeping the internal temperature less than 10° C. The mixture was placed in a 0° C. freezer overnight, and the solid was collected by vacuum filtration. The solid was washed with water (2×300 mL) and dried in vacuo at 42° C. overnight. The resulting (3R)-5-oxo-4-(phenylmethyl)-3-morpholinecarboxylic acid (72.20 g, 75%) was isolated as a white solid. LCMS: (M+H)⁺: 236.1.

### Part C

### (3R)-5-Oxo-N,4-bis(phenylmethyl)-3-morpholinecarboxamide

A mixture of (3R)-5-oxo-4-(phenylmethyl)-3-morpholinecarboxylic acid (69.67 g, 296.2 mmol) and 1-hydroxybenzotriazole (48.01 g, 355.4 mmol) in DCM (990 mL) was cooled to 0° C. To the mixture was added 4-methylmorpholine (163 mL, 1483 mmol), benzyl amine (35.6 mL, 325.9 mmol), and EDC (62.46 g, 325.8 mmol). The yellow solution was stirred overnight at room temperature, and was then washed with water (500 mL), 6 N aqueous HCl (300 mL), and water (200 mL). The organic phase was dried over anhydrous Na₂SO₄, filtered, and concentrated in vacuo to give crude (3R)-5-oxo-N,4-bis(phenylmethyl)-3-morpholinecarboxamide (97.05 g, >100% crude yield) as a yellow foam. LCMS: (M+H)⁺: 325.2.

### Part D

### 1-Phenyl-N-{[(3S)-4-(phenylmethyl)-3-morpholinyl]methyl}methanamine

To a 0° C. solution of (3R)-5-oxo-N,4-bis(phenylmethyl)-3-morpholinecarboxamide (assumed 96.07 g, 296.2 mmol) in PhMe (750 mL) was added Red-Al (65% w/w in PhMe, 645 mL) via addition funnel. After approximately 50 mL of Re—Al had been added, the resulting mixture was warmed to room temperature, and the remainder of the Red-Al was then added over 30 min. The mixture was then heated at 50° C. and stirred overnight. The solution was cooled to 0° C., and the reaction was quenched by the slow dropwise addition of 1 N aqueous NaOH (50 mL). An additional portion of 1 N aqueous NaOH (500 mL) was then added, followed by Et₂O (200 mL). The phases were separated, and the organic phase was washed with fresh 1 N aqueous NaOH (400 mL). The combined aqueous phase was extracted with fresh 4:1 PhMe-Et₂O (250 mL), and the combined organic phase was dried over anhydrous Na₂SO₄, filtered, and concentrated in vacuo to give 1-phenyl-N-{[(3S)-4-(phenylmethyl)-3-morpholinyl]methyl}methanamine as a yellow oil that was used without further purification. LCMS: (M+H)⁺: 297.1.

### Part E

### Ethyloxo((phenylmethyl){[(3S)-4-(phenylmethyl)-3 morpholinyl]methyl}amino)acetate

A solution of 1-phenyl-N-{[(3S)-4-(phenylmethyl)-3-morpholinyl]methyl}methanamine (assumed 87.79 g, 296.2 mmol) and N,N-diisopropylethylamine (67.1 mL, 385.2 mmol) in THF (1000 mL) was cooled to 0° C. To the solution was added ethyl chloro(oxo)acetate (36.3 mL, 326.2 mmol) dropwise via addition funnel. The resulting mixture was allowed to stir and warm to room temperature for 1 h. The solvent was then removed in vacuo to approximately 20% volume, and the residue was partitioned between EtOAc (600 mL), water (100 mL) and sat. aqueous NaHCO₃ (500 mL). The aqueous phase was extracted with a fresh portion of EtOAc (200 mL), and the combined organic phase was dried over anhydrous Na₂SO₄, filtered, and concentrated in vacuo. The residue was then azeotroped with EtOH (100 mL) to provide ethyl oxo((phenylmethyl){[(3S)-4-(phenylmethyl)-3-morpholinyl]methyl}amino)acetate as a yellow oil that was used without further purification. LCMS: (M+H)⁺: 397.2.

### Part F

### (9aS)-8-(Phenylmethyl)hexahydropyrazino[2,1-c][1,4]oxazine-6,7-dione

To a solution of ethyl oxo((phenylmethyl){[(3S)-4-(phenylmethyl)-3-morpholinyl]methyl}amino)acetate (assumed 117.43 g, 296.2 mmol) in EtOH (1000 mL) was added 10% Pd/C (23 g). The resulting mixture was hydrogenated under balloon pressure for 5 days, and then filtered through a glass fiber filter with EtOH washes. The solution was then concentrated in vacuo and crystallized from EtOH-EtOAc to give approximately 15 g of a white solid. The Pd/C filter cake was then slurried with MeOH (600 mL), and the mixture was filtered through a glass fiber filter with MeOH washes. The solution was then concentrated in vacuo and crystallized from EtOH-EtOAc to give a white solid that was combined with the initial batch of solid. The combined mother liquors were then concentrated in vacuo and crystallized from EtOH-EtOAc to give a white solid that was combined with the first two batches of solid to afford (9aS)-8-(phenylmethyl)hexahydropyrazino[2,1-c][1,4]oxazine-6,7-dione (39.97 g, 52% yield for 4 steps). LCMS: (M+H)⁺: 261.1.

### Part G

### (9aS)-8-(Phenylmethyl)octahydropyrazino[2,1-c][1,4]oxazine

To a 0° C. mixture of two combined batches of (9aS)-8-(phenylmethyl) hexahydropyrazino[2,1-c][1,4]oxazine-6,7-dione (combined total 42.29 g, 162.5 mmol) in Et₂O (406 mL) was added 1 M LiAlH₄ in Et₂O (406 mL, 406 mmol) via dropping funnel over 40 min. The mixture was then warmed to 35° C. and stirred for 6 days. The mixture was then cooled to 0° C., and EtOAc (100 mL) was slowly added, followed by water (20 mL), 15% aqueous NaOH (20 mL), and water (60 mL). The mixture was vigorously stirred for 1 h, and then diluted with EtOAc (500 mL). The mixture was filtered, and the filter cake was diluted with 1 N aqueous NaOH (500 mL) and extracted with Et₂O (2×200 mL). The combined organic phase (filtrate and Et₂O extractions) was dried over anhydrous Na₂SO₄, filtered, concentrated in vacuo, azeotroped with MeOH (100 mL), and dried overnight under high vacuum. The resulting colorless oil was combined with a second batch of product prepared in the same fashion from (9aS)-8-(phenylmethyl)hexahydropyrazino[2,1-c][1,4]oxazine-6,7-dione (0.3047 g, 1.1 mmol) to give crude (9aS)-8-(phenylmethyl)octahydropyrazino[2,1-c][1,4]oxazine (combined total 38.59 g, >100% crude yield). LCMS: (M+H)⁺: 233.1.

### Part H

### (9aS)-Octahydropyrazino[2,1-c][1,4]oxazine dihydrochloride

To a solution of (9aS)-8-(phenylmethyl)octahydropyrazino[2,1-c][1,4]oxazine (assumed 38.02 g, 163.6 mmol) in MeOH (330 mL) was added 6 N aqueous HCl (55 mL, 330 mmol) and 10% Pd/C (3.80 g). The mixture was hydrogenated overnight, and then filtered through a glass fiber filter. The filter cake was washed with MeOH, and the combined solution was concentrated in vacuo and azeotroped with MeOH (4×150 mL) to provide (9aS)-octahydropyrazino[2,1-c][1,4]oxazine dihydrochloride (34.78 g, 99% yield for 2 steps) as a red oil that solidified under high vacuum. LCMS: (M+H)⁺: 142.9.

### Part I

### (9aS)-8-(6-Chloro-5-fluoro-2-methyl-4-pyrimidinyl)octahydropyrazino[2,1-c][1,4]oxazine

To a mixture of (9aS)-octahydropyrazino[2,1-c][1,4]oxazine dihydrochloride (23.28 g, 108.2 mmol) in DCM (360 mL) was added 4,6-dichloro-5-fluoro-2-methylpyrimidine (19.59 g, 108.2 mmol) and N,N-diisopropylethylamine (68 mL, 390.4 mmol). The mixture was stirred for 2 h, and the resulting solution was diluted with DCM (100 mL) and washed with saturated aq. NaHCO₃ (200 mL). The aqueous phase was extracted with a fresh portion of DCM (100 mL), and this organic phase was washed with saturated aq. NaHCO₃ (50 mL). The combined organic phase was dried over anhydrous Na₂SO₄, filtered, and concentrated in vacuo to give (9aS)-8-(6-chloro-5-fluoro-2-methyl-4-pyrimidinyl)octahydropyrazino[2,1-c][1,4]oxazine as a light yellow oil that was used without further purification. LCMS: (M+H)⁺: 287.1.

### Part J

### (9aS)-8-(5-Fluoro-6-hydrazino-2-methyl-4-pyrimidinyl)octahydropyrazino[2,1-c][1,4]oxazine

To a solution of (9aS)-8-(6-chloro-5-fluoro-2-methyl-4-pyrimidinyl)octa hydro pyrazino[2,1-c][1,4]oxazine (assumed 31.03 g, 108.2 mmol) in dioxane (430 mL) was added hydrazine monohydrate (31 mL). The mixture was heated and stirred at 80° C. overnight, and then at 85° C. for 7 h. The mixture was cooled to room temperature and concentrated in vacuo. The residue was dissolved in DCM (500 mL) and washed with saturated aq. NaHCO₃ (200 mL). The aqueous phase was extracted with a fresh portion of DCM (100 mL), and this organic phase was washed with saturated aq. NaHCO₃ (100 mL). The combined organic phase was dried over anhydrous Na₂SO₄, filtered, concentrated in vacuo, and dried under high vacuum overnight to provide (9aS)-8-(5-fluoro-6-hydrazino-2-methyl-4-pyrimidinyl)octahydropyrazino[2,1-c][1,4]oxazine (27.98 g, 92% yield for 2 steps) as a light yellow solid. LCMS: (M+H)⁺: 283.3.

### Part K

### [(2R)-2-(Cyclopentyl methyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl][(phenyl methyl)oxy]formamide

To a solution of (2R)-3-cyclopentyl-2-({formyl[(phenylmethyl)oxy]amino}methyl)propanoic acid, N,N-diisopropylethylamine salt, isopropanol solvate (33.64 g, 68.0 mmol) in DMF (230 mL) was added (9aS)-8-(5-fluoro-6-hydrazino-2-methyl-4-pyrimidinyl)octahydropyrazino[2,1-c][1,4]oxazine (20.16 g, 71.4 mmol), N-methylmorpholine (30 mL, 273 mmol), 1-hydroxy-7-azabenzotriazole (11.10 g, 81.6 mmol), and EDC (15.64 g, 81.6 mmol). The solution was stirred overnight and then diluted with Et₂O (500 mL). The mixture was washed with water (2×200 mL), and the combined aqueous phase was extracted with a fresh portion of Et₂O (100 mL). This Et₂O phase was then washed with water (50 mL). This extraction—wash procedure was repeated 6 times, and the total combined organic phase was then diluted with DCM (250 mL). The organic phase was then dried over anhydrous Na₂SO₄, filtered, and concentrated in vacuo to give crude [(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl][(phenylmethyl)oxy]formamide (42.32 g, >100% crude yield) as a light yellow foam. LCMS: (M+H)⁺: 570.3.

### Part L

### [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide

To a solution of [(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl][(phenylmethyl)oxy]formamide (assumed 38.74 g, 68.0 mmol) in methanol (225 mL) was added 10% Pd/C (5.81 g). The mixture was hydrogenated under balloon pressure for 4 h, and was then filtered through a glass fiber filter with MeOH washes. The resulting solution was concentrated in vacuo to approximately 10% volume, diluted with EtOAc (400 mL), and concentrated in vacuo to approximately 30% volume. The resulting solid was collected by vacuum filtration and washed with EtOAc. The mother liquor and EtOAc washings were concentrated in vacuo to approximately 10% volume, and the resulting solid was collected by vacuum filtration and washed with EtOAc. The two crops of solid were combined and dried at 50° C. for 16 h under high vacuum to afford [(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (25.48 g, 78% yield for 2 steps) as a white solid. LCMS: (M+H)⁺: 480.1.

### Alternative Procedure

To a solution of crude [(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl][(phenylmethyl)oxy]formamide (assumed 39.73 g, 69.74 mmol) in MeOH (350 mL) was added 10% Pd/C (50% water, 7.9 g). The suspension was hydrogenated under balloon pressure for 3 h, and was then filtered through two glass fiber filters with MeOH washings. The resulting solution was concentrated in vacuo to a volume of approximately 70 mL, and was then diluted with EtOAc (500 mL). The solution was concentrated in vacuo to remove approximately 100 mL of solvent. The resulting solid was collected by vacuum filtration, and washed well with EtOAc followed by hexanes. The mother liquor was concentrated in vacuo, and then diluted with EtOAc (200 mL). The mixture was concentrated in vacuo to approximately 50% volume, and the resulting solid was collected by vacuum filtration and washed well with EtOAc followed by hexanes. The two batches of solid were combined and placed under high vacuum overnight. To this material was then added approximately 466 mg of material prepared through a similar sequence, and the combined batch was heated at 50° C. under high vacuum overnight to afford [(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (28.27 g, 83% yield for 2 steps). LC/MS: (M+H)⁺: 480.3. The resulting solid was analytically characterized and found to be polymorphic form, Form 1.

### Example 2

### [(2R)-3-(2-{2-Chloro-5-fluoro-6-[(9aS)-hexahydropyrazino[2,1-c][1,4]oxazin-8(1H)-yl]-4-pyrimidinyl}hydrazino)-2-(cyclopentylmethyl)-3-oxopropyl]hydroxyformamide

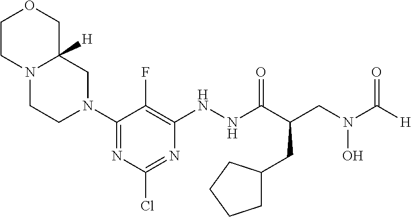 [SMILES: O=CN(O)C[C@@H](CC1CCCC1)C(=O)NNc1nc(Cl)nc(N2CCN3CCOC[C@@H]3C2)c1F]

[(2R)-3-(2-{2-Chloro-5-fluoro-6-[(9aS)-hexahydropyrazino[2,1-c][1,4]oxazin-8(1H)-yl]-4-pyrimidinyl}hydrazino)-2-(cyclopentylmethyl)-3-oxopropyl]hydroxyformamide was prepared according to General Procedure E, utilizing (9aS)-octahydropyrazino[2,1-c][1,4]oxazine dihydrochloride (which may be prepared as described in Example 24, Parts A-H) in place of isopropyl amine in Part A, utilizing 2N HCl in ether in Part B, performing an extractive (ether/water) workup rather than HPLC purification in Part C, and purifying the final product in Part D by recrystallization from EtOAc/ether rather than HPLC. LCMS: (M+H)⁺: 501.0.

### Example 3

### [(2R)-3-(2-{2-Chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl]hydroxyformamide

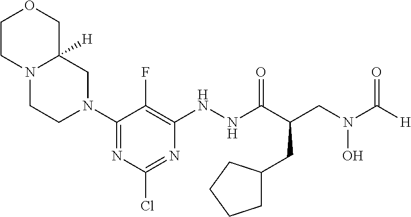 [SMILES: O=CN(O)C[C@@H](CC1CCCC1)C(=O)NNc1nc(Cl)nc(N2CCN3CCOC[C@H]3C2)c1F]

### Part A

### (3S)-5-Oxo-4-(phenylmethyl)-3-morpholinecarboxylic acid

To a 0° C. solution of N-benzylserine (19.15 g, 105.7 mmol) in 2 N aq. NaOH (100 mL) was added chloroacetyl chloride (10.2 mL, 126.4 mmol) dropwise, and the solution was stirred for 45 min. To the solution was added 30% aq. NaOH (40 mL) dropwise, and the reaction was stirred for 1 h, and then stirred and warmed to room temperature for 72 h. The reaction was adjusted to pH 1 and extracted with three portions of EtOAc. The combined organic phase was dried over anhydrous Na₂SO₄, filtered, and concentrated in vacuo. The residue was washed with EtOAc/hexane to give (3S)-5-oxo-4-(phenylmethyl)-3-morpholinecarboxylic acid (4.61 g, 19% yield).

### Part B

### (3S)-5-Oxo-N,4-bis(phenylmethyl)-3-morpholinecarboxamide

To a solution of (3S)-5-oxo-4-(phenylmethyl)-3-morpholinecarboxylic acid (4.611 g, 19.60 mmol) in DCM (65 mL) was added benzylamine (2.6 mL, 23.80 mmol), N-methylmorpholine (11 mL, 100.0 mmol), 1-hydroxy-7-azabenzotriazole (3.20 g, 23.51 mmol), and EDC (4.51 g, 23.53 mmol). The solution was stirred overnight and then diluted with DCM (100 mL). The solution was washed with 6 N aq. HCl (2×100 mL), and the organic phase was dried over anhydrous MgSO₄, filtered, and concentrated in vacuo to provide crude (3S)-5-oxo-N,4-bis(phenylmethyl)-3-morpholinecarboxamide (6.454 g, >100% crude yield) as a pale yellow foam. LCMS: (M+H)⁺: 325.2.

### Part C

### 1-Phenyl-N-{[(3R)-4-(phenylmethyl)-3-morpholinyl]methyl}methanamine

To a solution of (3S)-5-oxo-N,4-bis(phenylmethyl)-3-morpholinecarboxamide (6.3109 g, 19.45 mmol) in 1,2-dimethoxyethane (100 mL) was added LiAlH₄ (2.6 g, 68.51 mmol). The mixture was heated at 100° C. and stirred for 6 h, and then cooled to 60° C. and stirred overnight. The mixture was cooled to 0° C. and quenched by slow addition of 1 N aq. NaOH (100 mL). The mixture was extracted with Et₂O (2×150 mL), and the combined organic phase was dried over anhydrous Na₂SO₄, filtered, and concentrated in vacuo. The residue was purified by gradient silica gel chromatography (1% to 5% MeOH in DCM) to give 1-phenyl-N-{[(3R)-4-(phenylmethyl)-3-morpholinyl]methyl}methanamine (3.8342 g, 66%) as a light yellow oil. LCMS: (M+H)⁺: 297.1.

### Part D

### Methyl oxo((phenylmethyl){[(3R)-4-(phenylmethyl)-3-morpholinyl]methyl}amino)acetate

To a solution of 1-phenyl-N-{[(3R)-4-(phenylmethyl)-3-morpholinyl]methyl}methanamine (3.3777 g, 11.40 mmol) in THF (114 mL) was added N,N-diisopropylethylamine (3 mL, 17.22 mmol). To the solution was added methyl chloro(oxo)acetate (1.15 mL, 12.50 mmol) dropwise via syringe, and the mixture was stirred for 3 h. The solvent was removed in vacuo, and the residue was dissolved in EtOAc (200 mL) and washed with sat. aq. NaHCO₃ followed by water. The organic phase was dried over anhydrous Na₂SO₄, filtered, and concentrated in vacuo to afford crude methyl oxo((phenylmethyl){[(3R)-4-(phenylmethyl)-3-morpholinyl]methyl}amino)acetate (4.6881 g, >100% crude yield) as a yellow oil. LCMS: (M+H)⁺: 383.1.

### Part E

### (9aR)-8-(Phenylmethyl)hexahydropyrazino[2,1-c][1,4]oxazine-6,7-dione

To a solution of crude methyl oxo((phenylmethyl){[(3R)-4-(phenylmethyl)-3-morpholinyl]methyl}amino)acetate (assumed 4.3583 g, 11.40 mmol) in MeOH (114 mL) was added 10% Pd/C (50% water, 870 mg). The mixture was hydrogenated under balloon pressure for 16 h, and then filtered through a 0.2 μm membrane. The solution was concentrated in vacuo, and the residue was triturated with 20% EtOAc-hexanes. The resulting solid was collected by vacuum filtration and washed with hexanes to provide (9aR)-8-(phenylmethyl)hexahydropyrazino[2,1-c][1,4]oxazine-6,7-dione (2.5610 g, 86% for 2 steps) as a light yellow solid. LCMS: (M+H)⁺: 261.1.

### Part F

### (9aR)-8-(Phenylmethyl)octahydropyrazino[2,1-c][1,4]oxazine

To a 0° C. solution of (9aR)-8-(phenylmethyl)hexahydropyrazino[2,1-c][1,4]oxazine-6,7-dione (2.5610 g, 9.84 mmol) in THF (100 mL) was added LiAlH₄ (1.12 g, 29.51 mmol) portionwise. The mixture was heated at 70° C. and stirred for 1 week. The mixture was then cooled to 0° C. and quenched by addition of Na₂SO₄.10H₂O (2 g) followed by 1 N aq. NaOH (100 mL). The mixture was extracted with Et₂O (2×150 mL), and the combined organic phase was dried over anhydrous Na₂SO₄, filtered, and concentrated in vacuo. The residue was dissolved in DCM (200 mL), dried over anhydrous Na₂SO₄, filtered, and concentrated in vacuo. The residue was purified by gradient silica gel chromatography (0% to 100% EtOAc in hexanes; 1% Et₃N) to give (9aR)-8-(phenylmethyl)octahydropyrazino[2,1-c][1,4]oxazine (1.8147 g, 79%) as a colorless oil. LCMS: (M+H)⁺: 233.1.

### Part G

### (9aR)-Octahydropyrazino[2,1-c][1,4]oxazine dihydrochloride

To a solution of (9aR)-8-(phenylmethyl)octahydropyrazino[2,1-c][1,4]oxazine (1.8055 g, 7.77 mmol) in MeOH (80 mL) was added 1 N aq. HCl (15.5 mL, 15.5 mmol) and 10% Pd/C (50% water, 360 mg). The mixture was hydrogenated under balloon pressure overnight, and was then filtered through a 0.2 μm PTFE membrane. The solution was concentrated in vacuo and the residue was azeotroped with MeOH (3×50 mL) to provide crude (9aR)-octahydropyrazino[2,1-c][1,4]oxazine dihydrochloride (1.7185 g, >100% crude yield) as an orange solid. LCMS: (M+H)⁺: 142.9.

### Part H

### Tris(1,1-dimethylethyl) 2-{2-chloro-5-fluoro-6-[(9aR)-hexahydropyrazino[2,1-c][1,4]oxazin-8(1H)-yl]-4-pyrimidinyl}-1,1,2-hydrazinetricarboxylate

To a solution of crude (9aR)-octahydropyrazino[2,1-c][1,4]oxazine dihydrochloride (1.7185 g, 7.99 mmol) in DMF (40 mL) was added tris(1,1-di methylethyl) 2-(2,6-dichloro-5-fluoro-4-pyrimidinyl)-1,1,2-hydrazinetricarboxylate (3.97 g, 7.98 mmol) and N,N-diisopropylethylamine (4.60 mL, 26.41 mmol). The solution was stirred overnight and then diluted with Et₂O (200 mL). The mixture was washed with water (2×100 mL), and the combined aqueous phase was extracted with a fresh portion of Et₂O (100 mL). The combined organic phase was washed with a fresh portion of water (50 mL), and then dried over anhydrous Na₂SO₄. The mixture was filtered, concentrated in vacuo, and the residue was dissolved in DCM (200 mL) and dried over anhydrous Na₂SO₄. The mixture was filtered, and the solution was concentrated in vacuo. The residue was purified by gradient silica gel chromatography (0% to 100% EtOAc in hexanes; 1% Et₃N) to afford tris(1,1-dimethylethyl) 2-{2-chloro-5-fluoro-6-[(9aR)-hexahydropyrazino[2,1-c][1,4]oxazin-8(1H)-yl]-4-pyrimidinyl}-1,1,2-hydrazinetricarboxylate (4.34 g, 90%) as a white foam. LCMS: (M+H)⁺: 603.3.

### Part I

### (9aR)-8-(2-Chloro-5-fluoro-6-hydrazino-4-pyrimidinyl)octahydropyrazino[2,1-c][1,4]oxazine

To a solution of tris(1,1-dimethylethyl) 2-{2-chloro-5-fluoro-6-[(9aR)-hexahydropyrazino[2,1-c][1,4]oxazin-8(1H)-yl]-4-pyrimidinyl}-1,1,2-hydrazinetricarboxylate (4.34 g, 7.20 mmol) in MeOH (18 mL) was added 4 N HCl in dioxane (18 mL, 72 mmol). The solution was stirred for 3 days, and then concentrated in vacuo. The residue was dissolved in water (50 mL) and the solution was adjusted to pH 10 with 20% aq. K₂CO₃. The mixture was extracted with DCM (2×100 mL), and the combined organic phase was dried over anhydrous Na₂SO₄, filtered, and concentrated in vacuo to give (9aR)-8-(2-chloro-5-fluoro-6-hydrazino-4-pyrimidinyl)octahydropyrazino[2,1-c][1,4]oxazine (1.41 g, 65%) as an orange solid. LCMS: (M+H)⁺: 303.1.

### Part J

### [(2R)-3-(2-{2-Chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl][(phenylmethyl)oxy]formamide

To a solution of (9aR)-8-(2-chloro-5-fluoro-6-hydrazino-4-pyrimidinyl)octahydro pyrazino[2,1-c][1,4]oxazine (1.41 g, 4.66 mmol) in DMF (45 mL) was added (2R)-3-cyclopentyl-2-({formyl[(phenylmethyl)oxy]amino}methyl)propanoic acid (1.36 g, 4.45 mmol), N-methylmorpholine (2.45 mL, 22.3 mmol), 1-hydroxy-7-azabenzotriazole (0.730 g, 5.364 mmol), and EDC (1.02 g, 5.32 mmol). The solution was stirred overnight and was then diluted with Et₂O (200 mL). The mixture was washed with water (2×100 mL), and the combined aqueous phase was extracted with a fresh portion of Et₂O (100 mL). This Et₂O layer was washed with a fresh portion of water (50 mL), and the combined organic phase was dried over anhydrous Na₂SO₄, filtered, and concentrated in vacuo. The residue was dissolved in DCM (200 mL), dried over anhydrous Na₂SO₄, filtered, and concentrated in vacuo. The residue was azeotroped with MeOH (50 mL) to give [(2R)-3-(2-{2-chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl][(phenylmethyl)oxy]formamide (2.4027 g, 91% crude yield) as a red/orange oil. LCMS: (M+H)⁺: 590.2.

### Part K

### [(2R)-3-(2-{2-Chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl]hydroxyformamide

To a solution of [(2R)-3-(2-{2-chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl][(phenylmethyl)oxy]formamide (2.3947 g, 4.058 mmol) in MeOH (40 mL) was added 20% Pd(OH)₂/C (50% water, 240 mg). The mixture was hydrogenated under balloon pressure for 2.5 h, and then filtered through a 0.2 μm membrane. The solution was concentrated in vacuo, and the residue was purified by Gilson RPLC (10% MeCN in water to 65% MeCN in water; 8 min gradient). The desired fractions were combined, and the MeCN was removed in vacuo. The resulting mixture was extracted with EtOAc (2×150 mL), and the combined organic phase was dried over anhydrous Na₂SO₄, filtered, and concentrated in vacuo. The residue was then crystallized from EtOAc-hexanes to provide [(2R)-3-(2-{2-chloro-5-fluoro-6-[(9aR)-hexahydropyrazino[2,1-c][1,4]oxazin-8(1H)-yl]-4-pyrimidinyl}hydrazino)-2-(cyclopentylmethyl)-3-oxopropyl]hydroxyformamide (1.2759 g, 63%) as a pale pink solid. LCMS: (M+H)⁺: 500.1.

### Example 4

### [(2R)-2-(Cyclopentyl methyl)-3-(2-{5-fluoro-2-(fluoromethyl)-6-[(9aS)-hexahydropyrazino[2,1-c][1,4]oxazin-8(1H)-yl]-4-pyrimidinyl}hydrazino)-3-oxopropyl]hydroxyformamide

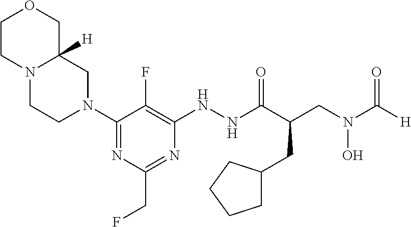 [SMILES: O=CN(O)C[C@@H](CC1CCCC1)C(=O)NNc1nc(CF)nc(N2CCN3CCOC[C@@H]3C2)c1F]

[(2R)-2-(Cyclopentylmethyl)-3-(2-{5-fluoro-2-(fluoromethyl)-6-[(9aS)-hexahydro pyrazino[2,1-c][1,4]oxazin-8(1H)-yl]-4-pyrimidinyl}hydrazino)-3-oxopropyl]hydroxyformamide was prepared according to General Procedure A, utilizing (9aS)-octahydropyrazino[2,1-c][1,4]oxazine dihydrochloride (Example 22) in place of pyrrolidine, using 4,6-dichloro-5-fluoro-2-(fluoromethyl)pyrimidine in place of 4,6-dichloro-5-fluoro-2-methylpyrimidine, and using 3 equivalents of DIPEA in Part A. LCMS: (M+H)⁺: 498.3.

### Example 5

### ((2R)-2-(Cyclopentylmethyl)-3-{2-[5-fluoro-6-[(9aS)-hexahydropyrazino[2,1-c][1,4]oxazin-8(1H)-yl]-2-(methylthio)-4-pyrimidinyl]hydrazino}-3-oxopropyl)hydroxyformamide

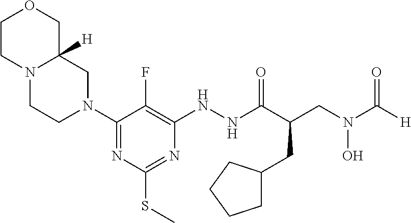 [SMILES: CSc1nc(NNC(=O)[C@H](CC2CCCC2)CN(O)C=O)c(F)c(N2CCN3CCOC[C@@H]3C2)n1]

((2R)-2-(Cyclopentylmethyl)-3-{2-[5-fluoro-6-[(9aS)-hexahydropyrazino[2,1-c][1,4]oxazin-8(1H)-yl]-2-(methylthio)-4-pyrimidinyl]hydrazino}-3-oxopropyl)hydroxyformamide was prepared according to General Procedure C, utilizing (9aS)-octahydropyrazino[2,1-c][1,4]oxazine dihydrochloride (Example 22) in place of azetidine hydrochloride in Part A. LCMS: (M+H)⁺512.2.

### Example 6

### (2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl form amide methanesulphonate Form 1

Crystalline[(2R)-2-(Cyclopentylmethyl)-3-(2-{5-fluoro-6-[(9aS)-hexahydro pyrazino[2,1-c][1,4]oxazin-8(1H)-yl]-2-methyl-4-pyrimidinyl}hydrazino)-3-oxopropyl]hydroxyformamide (500 mg) was dissolved in tetrahydrofuran (THF, 5 mL). Ethyl acetate (20 mL) was added to the solution. Separately, a solution of methanesulphonic acid (100 mg) in ethyl acetate (10 mL) was prepared. The methanesulfonic acid solution was added dropwise over a few minutes to the solution of freebase in tetrandrofuran and ethyl acetate. A precipitate was formed. The slurry was stirred with low heat (40° C. to 50° C.) for several hours. The precipitate's crystallinity was verified by polarized light microscopy. The precipitate and supernatant were separated by filtration. The solids were washed with ethyl acetate. The solids were then vacuum dried. The resulting solid was analytically characterized and found to be Form 1 of the methanesulphonate salt of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide by NMR (FIGS. 1 and 2, Tables 1 and 2) and XRPD (FIG. 3 and Table 3).

### Proton (¹H) and Carbon (¹³C) Nuclear Magnetic Resonance Spectroscopy of [(2R)-2-(Cyclopentylmethyl)-3-(2-{5-fluoro-6-[(9aS)-hexahydropyrazino-2,1-c][1,4]oxazin-8(1H)-yl]-2-methyl-4-pyrimidinyl}hydrazino)-3-oxopropyl]hydroxyformamide methanesulphonate Form 1

The 400 MHz ¹H NMR spectrum and the 101 MHz ¹³C NMR spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate in DMSO-d₆ (33.3 mg/mL at 25° C.) are presented in FIG. 1 and FIG. 2, respectively. Interpretations of the spectra are given in Table 1 and Table 2, respectively with the numbering sequence shown in the structure below. The ¹H and ¹³C NMR spectra are concordant with the proposed structure.

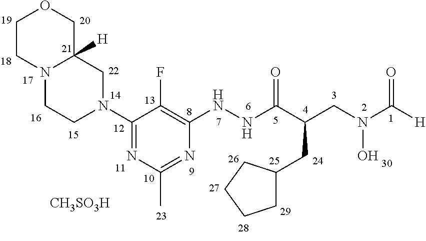 [SMILES: Cc1nc(NNC(=O)[C@H](CC2CCCC2)CN(O)C=O)c(F)c(N2CCN3CCOC[C@@H]3C2)n1 ; "CH3SO3H"]

TABLE 1
Interpretation of the ¹H NMR Spectrum of [(2R)-2-(Cyclopentylmethyl)-
3-(2-{5-fluoro-6-[(9aS)-hexahydropyrazino[2,1-c][1,4]oxazin-
8(1H)-yl]-2-methyl-4-pyrimidinyl} hydrazino)-3-oxopropyl]hydroxy-
formamide methanesulphonate Form 1 in DMSO-d₆

| Chemical | Multi- | No. of | |
|---|---|---|---|
| Shift (δ)* | plicity | H Atoms | Assignment |
| 1.02-1.10 | m (each) | 2 | 26a/29a, 26′a/29′a |
| 1.19-1.28 | m (each) | 1 | 24a and 24′a |
| 1.47-1.71 | overlapped | 6 | 27a/28a, 27b/28b, |
| | | | 24b, 24′b, 26b/29b |
| 1.91-2.00 | overlapped | 2 | 25, 25′, 26′b/29′b |
| 2.23 | s | 1.5 (rotamer) | 23′ |
| 2.26 | s | 1.5 (rotamer) | 23 |
| 2.38 | s | 3 (counterion) | Mesylate |
| 2.68-2.82 | m (each) | 1 | 4 and 4′ |
| 3.00 | t | 1 | 22a |
| 3.24-3.57 | overlapped | 8.5 (rotamer) | 153, 3′a, 3, 16, |
| | | | 18, 21, 20a |
| 3.72-3.80 | m (each) | 1.5 (rotamer) | 3′b, 19a |
| 4.00-4.08 | m (each) | 2 | 19b, 20b |
| 4.30 | d | 1 | 22b |
| 4.38 | d | 1 | 15b |
| 7.67 | s | 0.5 (rotamer) | 1 |
| 8.31 | s | 0.5 (rotamer) | 1′ |
| 9.02 | s | 0.5 (rotamer) | 7 |
| 9.05 | s | 0.5 (rotamer) | 7′ |
| 9.92 | s | 0.5 (rotamer) | 6 |
| 9.96 | s | 0.5 (rotamer) | 6′ |
| 10.32 | br | 2 | Exchangeable** |

TABLE 2
Interpretation of the ¹³C NMR Spectrum of [(2R)-2-
(Cyclopentylmethyl)-3-(2-{5-fluoro-6-[(9aS)-hexahydropyrazino[2,1-
c][1,4]oxazin-8(1H)-yl]-2-methyl-4-pyrimidinyl}
hydrazino)-3-oxopropyl]hydroxyformamide methanesulphonate
Form 1 in DMSO-d₆

| Chemical | Coupling Constant/ | |
|---|---|---|
| Shift (δ)* | J_{13C-19F}(Hz) | Assignment |
| 24.7 | — | 27/28 |
| 24.9 | — | 23 |
| 31.6 | — | 26′/29′ |
| 32.9 | — | 26/29 |
| 35.5 | — | 24′ |
| 35.6 | — | 24 |
| 36.9 | — | 25 |
| 37.0 | — | 25′ |
| 39.8 | — | Mesylate |
| 41.2 | — | 4 |
| 43.4 | br | 15 |
| 43.8 | br | 22 |
| 48.9 | — | 3′ |
| 51.4 | — | 16 |
| 51.7 | — | 18 |
| 52.0 | — | 3 |
| 60.1 | — | 21 |
| 63.7 | — | 19 |
| 65.2 | — | 20 |
| 130.3 | 248 (d) | 13′ |
| 130.4 | 248 (d) | 13 |
| 148.2 | — | 12 |
| 152.5 | 10 (d) | 8 |
| 157.4 | — | 1 |
| 160.1 | m | 10 and 10′ |
| 162.0 | — | 1′ |
| 172.7 | — | 5 |
| 172.8 | — | 5′ |

### X-Ray Powder Diffraction of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1

The XRPD pattern for polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate is presented in FIG. 3.

The peak list (Table 3) was produced by taking the 1^{st} four diffraction peaks followed by the six most intense peaks from the remaining peak list that were clearly defined peaks and not peak shoulders. The X-ray diffraction data was collected with copper K alpha radiation which is composed of K alpha 1 and K alpha 2. Each diffraction peak was profile fitted and the results for the K alpha 1 component are reported. The estimated uncertainity in peak position is +/−0.3 deg 2theta.

The peak list of 10 can vary somewhat with repeated measurements due to preferred orientation causing variation in the relative intensity of the peaks.

TABLE 3
XRPD Peak Positions for Polymorphic Form 1 of [(2R)-2-(Cyclopentyl-
methyl)-3-(2-{5-fluoro-6-[(9aS)-hexahydropyrazino[2,1-
c][1,4]oxazin-8(1H)-yl]-2-methyl-4-pyrimidinyl}hydrazino)-
3-oxopropyl]hydroxy-formamide methanesulphonate based on copper
K-alpha 1 radiation
2θ Peak Position ± 0.3 (2θ)

5.3
9.7
10.8
11.4
13.5
14.9
17.8
18.9
21.2
22.1

### Infrared Spectrum

The attenuated total reflectance (ATR) infrared spectrum of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 is presented in FIG. 4. The interpretation of the infrared spectrum is given in Table 4. The absorption characteristics are concordant with the proposed structure.

TABLE 4
Interpretation of the ATR Infrared Spectrum of [(2R)-
2-(cyclopentylmethyl)-3-(2-{5-fluoro-6-[(9aS)-
hexahydropyrazino[2,1-c][1,4]oxazin-8(1h)-yl]-2-
methyl-4-pyrimidinyl}hydrazino)-3-oxopropyl]hydroxyformamide
methanesulphonate Form 1

| Peak Wavenumber | |
|---|---|
| (cm⁻¹) | Assignment |
| 3204 | N—H Stretch (hydrazide) and |
| | O—H Stretch (hydroxamide) |
| 2947, 2865 | C—H Stretch (aliphatic) |
| 2453 | N—H Stretch (quarternary amine) |
| 1657, 1670 | C═O Stretch |
| 1606 | C═C Stretch (pyrimidine) |
| 1583 | Amide II (hydrazide) and |
| | C═C Stretch (pyrimidine) |
| 1225 | C—F Stretch |
| 1174, 1151, 1130, 1112 | C—N Stretch |

### Thermal Analysis

Based on differential scanning calorimetry (DSC) seen in FIG. 5 and thermogravimetric analysis (TGA) seen in FIG. 6, polymorphic Form 1 of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate decomposes at ca. 180° C. under nitrogen.

### Dissolution Rate and Solubility

[(2R)-2-(cyclopentyl methyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide was triturated with a mortar and pestle. The resulting powder (20 g, 1.0 eq, basis) was charged to a 250 mL jacketed vessel. The vessel was equipped with an overhead stirrer which was set to 60 RPM. The jacket fluid was controlled to 23° C. A solution of purified water (125 mL) and methanesulphonic acid (4.0 g, 2.7 mL, 1.0 eq) was prepared. The aqueous methanesulphonic acid solution was added to the vessel containing [(2R)-2-(cyclopentyl methyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide. The resulting suspension was stirred continuously and monitored by turbidity and visual inspection. After 1 day, the turbidity and pH readings stabilized. The suspension was observed for over 7.1 days and complete dissolution not observed.

[(2R)-2-(cyclopentyl methyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate was triturated with a mortar and pestle. The resulting powder (24 g, 1.0 eq) was charged to a similar 250 mL jacketed vessel. The vessel was equipped with an overhead stirrer which was set to 60 RPM. The jacket fluid was controlled to 23° C. Purified water (125 mL) was added to the vessel containing [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate. The resulting suspension was stirred continuously and monitored by turbidity and visual inspection. A clear solution was observed visually and confirmed turbidimetrically at 3 minutes.

[(2R)-2-(cyclopentyl methyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate exhibits faster dissolution and enhanced solubility when compared to the free base when subjected to one equivalent of methanesulphonic acid and water at a concentration of 160 mg/mL.

The pH solubility curve can be seen in Table 5 below.

TABLE 5
The pH Solubility Curve.

| | | Solubility | |
|---|---|---|---|
| Solvent | pH | (mg/mL) | Input |
| 1M Methanesulphonic Acid | 3.42 | 347* | Free Base |
| 0.5M Methanesulphonic Acid | 3.39 | 193* | Free Base |
| 0.3M Methanesulphonic Acid | 3.34 | 116* | Free Base |
| 0.1M Methanesulphonic Acid | 3.57 | 41.7 | Free Base |
| 0.05M Methanesulphonic Acid | 3.76 | 22 | Free Base |
| 0.03M Methanesulphonic Acid | 3.98 | 13.9 | Free Base |
| 0.01M Methanesulphonic Acid | 4.33 | 4.98 | Free Base |
| 0.003M Methanesulphonic Acid | 4.86 | 1.93 | Free Base |
| Purified Water | 3.2 | >452** | Mesylate |

*The solubility behaviour above ca. pH 3.5 is typical of a weak base. Below ca. pH 3.5, (2R)-2-(cyclopentylmethyl)-3-(2-{5-fluoro-6-[(9aS)-hexahydropyrazino [2,1-c][1,4] oxazin-8(1h)-yl]-2-methyl-4-pyrimidinyl}hydrazino)-3-oxopropyl]hydroxylformamide methanesulphonate appears to self-associate and the solubility of (2R)-2-(cyclopentylmethyl)-3-(2-{5-fluoro-6-[(9aS)-hexahydropyrazino [2,1-c][1,4]oxazin-8(1h)-yl]-2-methyl-4-pyrimidinyl}hydrazino)-3-oxopropyl]hydroxylformamide methanesulphonate Form 1increases with no decrease in pH as more acid is added
**Equilibrium not reached

### Novel Use

Due to the increased solubility and/or dissolution rate, [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 can be formulated to improve the following attributes when compared to a lyophilized product of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide:
1) drug load per vial from 400 mg to 1200 mg (free base equivalent), 2) degradation rate on processing, storage (degradation rate of 1.7%/month for free base versus 0.2%/month for mesylate at forced conditions: 50° C., ambient humidity), and reconstitution (see Table 6), 3) pH at a set dosing concentration on presentation to the patient from a range of 3.0 to 3.2 for the free base to a range of 3.8 to 4.0 for the mesylate, 4) a direct powder fill option for the final product.

TABLE 6
Hydrolytic Degradation Rate of Reconstituted
Lyophile at 5 mg/mL

| | Storage | Degradation Rate |
|---|---|---|
| API input | Temperature | % hydrolized/day |
| Free base | 5° C. | 0.06 |
| Free base | 25° C. | 0.53 |
| Mesylate | 5° C. | 0.01 |
| Mesylate | 25° C. | 0.11 |

### Example 7

### Polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate

1-propanol (600 mL) was added to crystalline [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (100 g). The resulting slurry was warmed to 60° C. at a rate of 1.5° C. per minute. Methanesulfonic acid (13.54 mL) was added to the slurry. Complete dissolution was observed. The solution was filtered through filter paper. The dissolution vessel and filter paper was rinsed with 1-propanol (100 mL). The temperature of the solution was adjusted to 50° C. and [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate seed (1.0 g) was added to induce crystallization. The resulting slurry was aged for 1 hour at 50° C. then cooled to 20° C. at 0.1° C. per minute, then aged for 2 hours, and cooled to 0° C. at 0.1° C./min. The slurry was aged for approximately 3 hours at 0° C. The resulting solids and supernatant were separated by filtration at 0° C. The solids were rinsed with 1-propanol (100 mL). The solids were blown with nitrogen for 45 minutes. The solids were then vacuum dried at 50° C. for approximately 40 minutes. The resulting solid was analytically characterized and found to be Form 1.

### ¹³C Solid State Nuclear Magnetic Resonance (SSNMR) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form I

The ¹³C solid state Nuclear Magnetic Resonance (SSNMR) of crystalline anhydrate[(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide methanesulphonate polymorphic Form I is identified and characterized by the peak pattern substantially shown in FIG. 12. The ¹³C solid state nuclear magnetic resonance (SSNMR) spectrum was obtained on a spectrometer operating at a frequency of 100.56 MHz for ¹³C observation using a cross-polarization pulse sequence with a Bruker 4-mm triple resonance magic-angle spinning probe at a rotor frequency of 8 kHz

The ¹³C SSNMR spectrum of FIG. 12 is indentified by characteristic chemical peak shifts at about 176.47±0.2, 162.53±0.2, 160.63±0.2, 152.70±0.2, 147.69±0.2, 131.27±0.2* (i.e., *131.27 and 128.75 ppm arise from a single carbon site split by the ¹J_{C-F} coupling), 128.75±0.2*, 64.86±0.2, 56.15±0.2, 54.77±0.2, 52.22±0.2, 45.94±0.2, 42.51±0.2, 42.03±0.2, 37.96±0.2, 36.66±0.2, 33.27±0.2, 31.67±0.2, 25.50±0.2, and 22.32±0.2 ppm.

### ¹⁹F solid state nuclear magnetic resonance (SSNMR) spectrum of crystalline anhydrate[(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form I

¹⁹F solid state nuclear magnetic resonance (SSNMR) spectrum of crystalline anhydrate[(2R)-2-(cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide methanesulphonate polymorphic Form 1 is identified and characterized by the peak pattern substantially shown in FIG. 13. The ¹⁹F SSNMR spectrum was obtained on a spectrometer operating at a frequency of 376.21 MHz for ¹⁹F observation using a cross-polarization pulse sequence with a Bruker 4-mm triple resonance magic-angle spinning probe at a rotor frequency of 12.5 kHz. The¹⁹F SSNMR comprises an isotropic chemical shift at −166.32±0.2 ppm.

### Example 8

### [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide dimethanesulphonate

Crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (100 mg) was dissolved in ethyl acetate (10 mL). Methanesulphonic acid (40.1 mg) in ethyl acetate (ca. 10 mL) was prepared. The methanesulphonic acid solution was added dropwise to the solution of freebase in ethyl acetate. A precipitate was formed. The slurry was stirred and aged overnight. The precipitate's crystallinity was verified by polarized light microscopy. The precipitate and supernatant were separated by filtration. The solids were then vacuum dried. The resulting solid was analytically characterized and found to be Form 1 of the dimethanesulphonate salt of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide by XRPD (see FIG. 7).

### X-Ray Powder Diffraction of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate

The XRPD pattern for polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate is presented in FIG. 7. The X-ray powder diffractogram was collected with a diffraction system utilizing copper Kα radiation, automated divergent slits, nickel Kβ filter, and multiple strip detector. Sample presentation consisted of a thin powder layer mounted on a silicon zero background wafer.

### Thermal Analysis

Based on differential scanning calorimetry (DSC) seen in FIG. 8 and thermogravimetric analysis (TGA) seen in FIG. 9, polymorphic Form 1 of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide dimethanesulphonate decomposes at ca. 180 to 185° C. under nitrogen.

### Example 9

### [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide camphorsulfonate

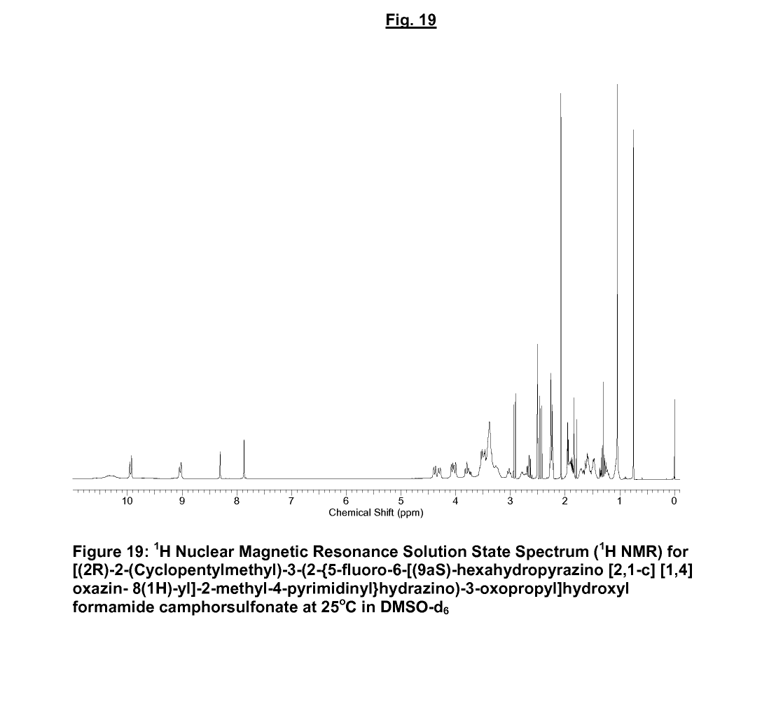

Crystalline[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (324.3 mg) was added to acetonitrile (MeCN, 3.2 mL). To the slurry, (7,7-dimethyl-2-oxo bicyclo[2.2.1]heptan-1-yl)methanesulfonic acid (camphorsulfonic acid, 171.8 mg) was added. The solids dissolved. The solution was warmed to 40° C. and aged for 2 hours then cooled to ambient temperature (ca. 23° C.) and aged overnight. Solids were observed and isolated by filtration to yield [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate (421.1 mg). The resulting solid was analytically characterized and found to be Form 1 of the camphorsulfonate salt of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide by NMR (see, FIG. 19) and XRPD (see, FIG. 10).

### ¹H Nuclear Magnetic Resonance Data (¹H NMR) for [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide camphorsulfonate

¹H NMR (400 MHz, DMSO-d₆, referenced to TMS=0.00 ppm, T=25C, rotamers present due to hindered rotation, major rotamers listed with integration rounded to nearest ½ units) δ ppm 10.6-9.0 (4H, several broad s), 8.30 (½H, s), 7.87 (½H, s), 4.40-4.28 (2H, several multiplet), 4.08-4.00 (2H, several m), 3.82-3.72 (3/2 H, several m), 3.56-3.23 (17/2 H, several m), 3.05-2.99 (1H, m), 2.92 (1H, d, J=15 Hz), 2.82-2.61 (2H, several m), 2.44 (1H, d, J=15 Hz), 2.28-2.21 (4H, several s and m), 2.00-1.79 (5H, several m), 1.72-1.47 (6H, several m), 1.36-1.20 (3H, several m), 1.10-1.00 (2H, several m), 1.04 (3H, s), 0.75 (3H, s) (see FIG. 19).

### X-Ray Powder Diffraction

The XRPD pattern for polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate is presented in FIG. 10. The X-ray powder diffractogram was collected with a diffraction system utilizing copper Kα radiation, automated divergent slits, nickel Kβ filter, and multiple strip detector. Sample presentation consisted of a thin powder layer mounted on a silicon zero background wafer.

### Thermal Analysis

Based on differential scanning calorimetry (DSC) seen in FIG. 11, polymorphic Form 1 of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide camphorsulfonate has an apparent melt onset of ca. 140° C. and decomposes at ca. 180° C. under nitrogen.

### Example 10

### Pharmaceutical Formulations of [(2R)-2-(cyclopentylmethyl)-3-(2-{5-fluoro-6-[(9aS)-hexahydropyrazino[2,1-c][1,4]oxazin-8(1 h)-yl]-2-methyl-4-pyrimidinyl}hydrazino)-3-oxopropyl]hydroxyformamide methanesulphonate Form 1 and Form 2

### Example 10A

### Pharmaceutical Formulation of [(2R)-2-(cyclopentylmethyl)-3-(2-{5-fluoro-6-[(9aS)-hexahydropyrazino[2,1-c][1,4]oxazin-8(1 h)-yl]-2-methyl-4-pyrimidinyl}hydrazino)-3-oxopropyl]hydroxyformamide methanesulphonate Form 1

The pharmaceutical formulation was prepared by dissolving a target concentration of 120 mg/mL of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 1 and 50 mg/mL of anhydrous mannitol in a vessel containing purified water. The vessel contents were stirred to obtain a solution, brought to final volume with purified water, and stirred again to ensure complete mixing. The solution was passed through a 0.2 μm sterilizing filter. A known quantity of the solution was then filled into glass vials and the vials were partially stoppered with rubber lyophilization stoppers. The vials were then placed into a tray-type lyophilizer and freeze dried. The lyophilizer was backfilled with dry nitrogen to just below atmospheric pressure (approximately 650 Torr) and the vials were fully stoppered. The vials were unloaded from the lyophilizer and sealed.

### Example 10 B

### Pharmaceutical Formulation of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 2

The pharmaceutical formulation was prepared by dissolving a target concentration of 120 mg/mL of [(2R)-2-(cyclopentylmethyl)-3-(2-{5-fluoro-6-[(9aS)-hexahydropyrazino[2,1-c][1,4]oxazin-8(1 h)-yl]-2-methyl-4-pyrimidinyl}hydrazino)-3-oxopropyl]hydroxyformamide methanesulphonate Form 2 and 50 mg/mL of anhydrous mannitol in a vessel containing purified water. The vessel contents were stirred to obtain a solution, brought to final volume with purified water, and stirred again to ensure complete mixing. The solution was passed through a 0.2 μm sterilizing filter. A known quantity of the solution was then filled into glass vials and the vials were partially stoppered with rubber lyophilization stoppers. The vials were then placed into a tray-type lyophilizer and freeze dried. The lyophilizer was backfilled with dry nitrogen to just below atmospheric pressure (approximately 650 Torr) and the vials were fully stoppered. The vials were unloaded from the lyophilizer and sealed.

### Example 11

### [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide methanesulfonate polymorphic Form 2

Crystalline[(2R)-2-(Cyclopentylmethyl)-3-(2-{5-fluoro-6-[(9aS)-hexahydro pyrazino[2,1-c][1,4]oxazin-8(1H)-yl]-2-methyl-4-pyrimidinyl}hydrazino)-3-oxo propyl]hydroxyformamide (15 g) was added to 1-Propanol (nPrOH, 240 mL) and Isooctane (60 mL). To the slurry, methanesulfonic acid (2.992 g) diluted in nPrOH (15 ml) was added at 10° C. The solids dissolved.

The solution was warmed to 35° C. and seeded with [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide methanesulfonate polymorphic Form 1. The batch was cooled to 10° C., resulting in concomitant nucleation of Form 2. The mixture of forms was heated to 35° C., and cooled to 10° C., and the solids were isolated by filtration to yield [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyl formamide methanesulfonate (14.32 g).

The resulting solid was analytically characterized and found to be Form 2 of the methansulfonate salt of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide by IR, NMR and XRPD (See, FIGS. 14 to 18).

### Infrared Spectrum

The attenuated total reflectance (ATR) infrared spectrum of [(2R)-2-(cyclopentylmethyl)-3-(2-{5-fluoro-6-[(9aS)-hexahydropyrazino[2,1-c][1,4]oxazin-8(1h)-yl]-2-methyl-4-pyrimidinyl}hydrazino)-3-oxopropyl]hydroxyformamide methanesulphonate Form 2 is presented in FIG. 14 and the interpretation of the infrared spectrum is given in Table 7. The absorption characteristics are consistent and correspond with the proposed structure.

TABLE 7
Interpretation of the ATR Infrared Spectrum of [(2R)-
2-(cyclopentylmethyl)-3-(2-{5-fluoro-6-[(9aS)-
hexahydropyrazino[2,1-c][1,4]oxazin-8(1h)-yl]-2-
methyl-4-pyrimidinyl}hydrazino)-3-oxopropyl]hydroxyformamide
methanesulphonate Form 2

| Peak Wavenumber | |
|---|---|
| (cm⁻¹) | Assignment |
| 3565, 3468, 2943 | N—H Stretch (hydrazide) |
| 3201 | O—H Stretch (alcohol) |
| 2863 | C—H Stretch (aliphatic) |
| 2626 | N—H Stretch (quarternary amine) |
| 1699, 1653 | C═O Stretch |
| 1611 | C═C Stretch (pyrimidine) |
| 1577 | Amide II (hydrazide) and |
| | C═C Stretch (pyrimidine) |
| 1347, 1329 | N—O Stretch |
| 1154, 1034 | C—F Stretch |
| 766 | C—H Stretch (pyrimidine) |

### X-Ray Powder Diffraction of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2

The XRPD pattern for [(2R)-2-(Cyclopentylmethyl)-3-(2-{5-fluoro-6-[(9aS)-hexahydro pyrazino[2,1-c][1,4]oxazin-8(1H)-yl]-2-methyl-4-pyrimidinyl}hydrazino)-3-oxopropyl]hydroxyl formamide methanesulphonate polymorphic Form 2 is presented in FIG. 15.

The peak list (Table 8) for Form 2 was produced by assigning degrees two theta (2θ) positions to the 10 strongest peaks in the region from 2 to 20 degrees two theta (2θ) from a capillary powder X-ray diffractogram. The X-ray diffraction data was collected with copper K alpha radiation which is composed of K alpha 1 and K alpha 2. Each diffraction peak was profile fitted and the results for the K alpha 1 component are reported. The estimated uncertainity in peak position is +/−0.3 deg 2theta.

As is conventionally understood by one skilled in the art, the relative intensities of such peaks may vary from sample to sample and preparation to preparation due to varying preferred orientation of sample particles in the sample holder and instrumentation calibration.

TABLE 8
XRPD Peak Positions for Polymorph of [(2R)-2-(Cyclopentylmethyl)-
3-(2-{5-fluoro-6-[(9aS)-hexahydropyrazino[2,1-c][1,4]oxazin-8(1H)-
yl]-2-methyl-4-pyrimidinyl}hydrazino)-3-oxopropyl]hydroxy-formamide
methanesulphonate Form 2 based on copper K-alpha 1 radiation
2θ Peak Position ± 0.3 (2θ)

5.5
9.3
9.7
10.8
13.6
14.5
15.0
16.2
17.8
19.6

### ¹H Nuclear Magnetic Resonance (¹H NMR) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2

¹H NMR (500 MHz, DMSO-d₆, referenced to TMS=0.00 ppm, T=25° C., rotamers present due to hindered rotation, major rotamers listed with integration rounded to nearest ½ units) δ ppm 10.6-9.7 (3H, several broad s), 9.06 (½H, s), 9.03 (½H, s), 8.30 (½H, s), 7.87 (½H, s), 4.38 (1H, d, J=13 Hz), 4.30 (1H, d, J=13 Hz), 4.08-4.00 (2H, several m), 3.81-3.72 (3/2 H, several m), 3.56-3.24 (17/2 H, several m), 3.04-3.00 (1H, m), 2.82-2.70 (1H, several m), 2.41 (3H, s), 2.26-2.23 (3H, several s), 1.99-1.90 (2H, several m), 1.71 (1H, broad m), 1.66-1.47 (5H, several m), 1.27-1.20 (1H, m), 1.08-1.02 (2H, several m). (see FIG. 16)

### ¹³C and ¹⁹F Solid State Nuclear Magnetic Resonance (SSNMR) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2

¹³C and ¹⁹F solid state NMR data, respectively, as described below, respectively, were acquired using a Bruker Avance 400 triple-resonance spectrometer operating at a ¹H frequency of 399.87 MHz.

The ¹³C SSNMR spectra shown were obtained using a cross-polarization pulse sequence with a Bruker 4-mm triple resonance magic-angle spinning probe at a rotor frequency of 8 kHz. A linear power ramp from 75 to 90 kHz was used on the ¹H channel to enhance cross-polarization efficiency. Spinning sidebands were eliminated by a five-pulse total sideband suppression pulse sequence. ¹H decoupling was obtained using the Spinal-64 sequence.

The ¹⁹F SSNMR spectra shown were obtained using a cross-polarization pulse sequence with a Bruker 4-mm triple resonance magic-angle probe at a rotor frequency of 12.5 kHz. Characteristic ¹³C and ¹⁹F NMR peak positions are reported relative to tetramethylsilane at 0 ppm (parts per million) and are quoted to a precision of +/−0.2 ppm, because of instrumental variability and calibration.

### ¹³C Solid State Nuclear Magnetic Resonance (SSNMR) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2

[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic crystalline anhydrate Form 2 was identified or characterized by ¹³C solid state nuclear magnetic resonance (SSNMR) spectrum as depicted by the peak pattern substantially shown in FIG. 17, obtained on a spectrometer operating at a frequency of 100.56 MHz for ¹³C observation using a cross-polarization pulse sequence with a Bruker 4-mm triple resonance magic-angle spinning probe at a rotor frequency of 8 kHz.

FIG. 17 shows an ¹³C SSNMR, which shows characteristic chemical peak shifts at 174.30±0.2, 161.44±0.2, 161.16±0.2, 160.86±0.2, 160.39±0.2 154.28±0.2, 153.01±0.2, 149.82±0.2, 132.12±0.2* 131.77±0.2*, 129.70±0.2*, 129.31±0.2* 68.30±0.2, 64.57±0.2, 63.83±0.2, 61.34±0.2, 59.89±0.2, 54.19±0.2, 53.81±0.2, 52.92±0.2, 52.43±0.2, 51.31±0.2, 50.16±0.2, 45.90±0.2, 42.86±0.2, 41.87±0.2, 40.22±0.2, 38.73±0.2, 37.75±0.2, 36.48±0.2, 35.59±0.2, 33.97±0.2, 32.82±0.2, 26.89±0.2, 26.23±0.2, 25.86±0.2, and 25.23±0.2 ppm. (i.e, where *132.12, 131.77, 129.70 and 128.75 ppm arise from two carbon sites, each split by the ¹J_{C-F} coupling).

### ¹⁹F solid state nuclear magnetic resonance (SSNMR) spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic Form 2

¹⁹F solid state nuclear magnetic resonance (SSNMR) spectrum of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate polymorphic crystalline anhydrate Form 2 is characterized and identified by the peak pattern substantially shown in FIG. 18, where the SSNMR spectrum was obtained on a spectrometer operating at a frequency of 376.21 MHz for ¹⁹F observation using a cross-polarization pulse sequence with a Bruker 4-mm triple resonance magic-angle spinning probe at a rotor frequency of 12.5 kHz.

FIG. 18 shows an ¹⁹F SSNMR with a characteristic isotropic chemical peak shifts at −166.56±0.2 and −171.26±0.2 ppm.

### Example 12

### Polymorphic of [(2R)-2-(Cyclopentylmethyl)-3-(2-{5-fluoro-6-[(9aS)-hexahydro pyrazino[2,1-c][1,4]oxazin-8(1H)-yl]-2-methyl-4-pyrimidinyl}hydrazino)-3-oxopropyl]hydroxyl formamide methanesulphonate Form 2

[(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide (33.8 g) was added to a 1 L jacketed lab reactor (“JLR”). N-propanol (473 mL) was added to the 1 L JLR. The contents were heated to 70° C. to dissolve. Complete dissolution was confirmed. The reactor was rinsed with n-propanol (68 mL). The contents were cooled to 25° C. at 0.25° C./min. Methanesulfonic acid solution (11.4 mL of 20% wt/wt of methanesulfonic acid in n-propanol) was charged to the JLR. [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulfonate polymorphic Form 2 (0.34 g) was added to the slurry. The slurry was aged for more than 30 minutes.

Methanesulfonic acid solution (45.5 mL of 20% wt/wt of methanesulfonic acid in n-propanol) was charged to the JLR at a linear, controlled rate over 6 hours. The resulting slurry was aged for 2 hours. To the slurry, isooctane (144 mL) was added at a controlled rate over 6 hours. The slurry was then cooled to 15° C. at a 0.1° C./min and aged for 1 hour. The resulting solids were Isolated by filtration.

The solids were washed with a mixture of n-propanol (100 mL) and isooctane (25 mL). The solids were then washed with isooctane (100 mL). The cake was blown with nitrogen for 2 hours and dried under vacuum at 50° C. for 48 hours yielding 34.6 g (85.1% molar yield) of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulfonate polymorphic Form 2 by XRPD (see, FIG. 20).

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

1. A compound which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate.

2. A compound according to claim 1 which is polymorphic Form 1 of [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate.

3. The compound according to claim 2 having a ¹H NMR Spectrum as shown in FIG. 1, a ¹³C NMR Spectrum as shown in FIG. 2, a Differential Scanning calorimetry (DSC) Spectrum as shown in FIG. 5, having a Thermogravimetric Analysis (TGA) Spectrum as shown in FIG. 6.

4. (canceled)

5. The compound according to claim 2 having an X-ray diffraction pattern which comprises characteristic peaks in degrees two-theta as shown in FIG. 3.

6. The compound according to claim 5 having characteristic peaks from 0° degrees 2-theta (2θ) to 55° degrees 2-theta (2θ) at about 5.3±0.3 (2θ), 9.7±0.3 (2θ), 10.8±0.3 (2θ), 11.4±0.3 (2θ), 13.5±0.3 (2θ), 14.9±0.3 (2θ), 17.8±0.3 (2θ). 18.9±0.3 (2θ). 21.2±0.3 (2θ) and 22.1±0.3 (2θ).

7. The compound according to claim 2 having an Attenuated Total Reflectance Infrared Spectrum which comprises characteristic absorption bands expressed in wave numbers as shown in FIG. 4.

8. The compound according to claim 7 having characteristic Infrared Spectrum peaks from 500 cm⁻¹ to 4000 cm⁻¹ wavenumbers at about 1174 cm⁻¹, 1151 cm⁻¹, 1130 cm⁻¹, 1112 cm⁻¹, 1225 cm⁻¹, 1583 cm⁻¹, 1606 cm⁻¹, 1657 cm⁻¹, 1670 cm⁻¹, 2453 cm⁻¹, 2947 cm⁻¹, 2865 cm⁻¹ and 3204 cm⁻¹.

9. (canceled)

10. (canceled)

11. A compound which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate.

12. The compound according to claim 11 having an X-ray diffraction pattern which comprises characteristic peaks in degrees two-theta as shown in FIG. 7, a Differential Scanning calorimetry (DSC) Spectrum as shown in FIG. 8 and a Thermogravimetric Analysis (TGA) Spectrum as shown in FIG. 9.

13. (canceled)

14. (canceled)

15. A compound which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulfonate.

16. The compound according to claim 15 having an X-ray diffraction pattern which comprises characteristic peaks in degrees two-theta as shown in FIG. 10 and a Differential Scanning calorimetry (DSC) Spectrum as shown in FIG. 11.

17. (canceled)

18. A pharmaceutical composition comprising a compound according to claim 1 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate and at least one pharmaceutically acceptable excipient.

19. The pharmaceutical composition according to claim 18, wherein [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate is Form 1 or Form 2.

20. The pharmaceutical composition according to claim 18 formulated for intravenous (iv) administration.

21. A pharmaceutical composition comprising a compound according to claim 11 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate and at least one pharmaceutically acceptable excipient.

22. (canceled)

23. A pharmaceutical composition comprising a compound according to claim 15 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphorsulphonate and at least one pharmaceutically acceptable excipient.

24. (canceled)

25. A method for treatment of a bacterial infection comprising administration of a therapeutically effective amount of a compound according to claim 1 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate to a human in need thereof.

26. The method for treatment of a bacterial infection according to claim 25, wherein [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate is selected from [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 or [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2.

27. The method according to claim 25 wherein the bacterial infection is caused by Streptococcus, Staphylococcus, Moraxella, Haemophilus, Neisseria, Mycoplasma, Legionella, Chlamydia, Bacteroides, Clostridium, Fusobacterium, Propionibacterium, or Peptostreptococcus.

28. The method according to claim 25 wherein the bacterial infection is an ear infection, sinusitis, upper respiratory tract infection, lower respiratory tract infection, genital infection, skin and soft tissue infection, or bacterial endocarditis.

29. A method for treatment of a bacterial infection comprising administration of a therapeutically effective amount of a compound according to claim 11 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide dimethanesulphonate to a human in need thereof.

30. The method according to claim 29 wherein the bacterial infection is caused by Streptococcus, Staphylococcus, Moraxella, Haemophilus, Neisseria, Mycoplasma, Legionella, Chlamydia, Bacteroides, Clostridium, Fusobacterium, Propionibacterium, or Peptostreptococcus.

31. The method according to claim 29 wherein the bacterial infection is an ear infection, sinusitis, upper respiratory tract infection, lower respiratory tract infection, genital infection, skin and soft tissue infection, or bacterial endocarditis.

32. A method for treatment of a bacterial infection comprising administration of a therapeutically effective amount of a compound according to claim 15 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide camphor sulphonate to a human in need thereof.

33. The method according to claim 32 wherein the bacterial infection is caused by Streptococcus, Staphylococcus, Moraxella, Haemophilus, Neisseria, Mycoplasma, Legionella, Chlamydia, Bacteroides, Clostridium, Fusobacterium, Propionibacterium, or Peptostreptococcus.

34. The method according to claim 32 wherein the bacterial infection is an ear infection, sinusitis, upper respiratory tract infection, lower respiratory tract infection, genital infection, skin and soft tissue infection, or bacterial endocarditis.

35. The compound according to claim 2, wherein the compound (Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 is a crystalline anhydrate or crystalline anhydrous form, a hydrate, or a mixture thereof.

36. The compound according to claim 35, wherein the compound (Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 is a crystalline anhydrate or crystalline anhydrous form.

37. The compound according to claim 36, having a ¹³C Solid State NMR(SSNMR) Spectrum as shown in FIG. 12 and a ¹⁹F Solid State NMR (¹⁹F SSNMR) spectrum shown in FIG. 13.

38. (canceled)

39. A pharmaceutical composition which comprises a compound according to claim 2 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 1 which is a crystalline anhydrate or crystalline anhydrous form.

40. A compound which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2.

41. The compound according to claim 40, wherein the compound [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 is a crystalline anhydrate or crystalline anhydrous form, a hydrate, or a mixture thereof.

42. The compound according to claim 41, wherein the compound [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 is a crystalline anhydrate or crystalline anhydrous form.

43. The compound according to claim 40 having a ¹H NMR Spectrum as shown in FIG. 16, a ¹³C Solid State NMR Spectrum as shown in FIG. 17 and a ¹⁹F Solid State NMR Spectrum as shown FIG. 18.

44. (canceled)

45. The compound according to claim 40 having an X-ray diffraction pattern which comprises characteristic peaks in degrees two-theta as shown in FIG. 15.

46. The compound according to claim 45 having characteristic peaks from 0° degrees 2-theta (2θ) to 55° degrees 2-theta (2θ) at about 5.5±0.3 (2θ), 9.3±0.3 (2θ), 9.7±0.3 (2θ), 10.8±0.3 (2θ), 13.6±0.3 (2θ), 14.5±0.3 (2θ), 15.0±0.3 (2θ). 16.2±0.3 (2θ), 17.8±0.3 (2θ) and 19.6±0.3 (2θ).

47. The compound according to claim 40 having an Attenuated Total Reflectance Infrared Spectrum which comprises characteristic absortion bands expressed in wave numbers as shown in FIG. 14.

48. The compound according to claim 47 having characteristic Infrared Spectrum peaks from 500 cm⁻¹ to 4000 cm⁻¹ wavenumbers at about 766 cm⁻¹, 1034 cm⁻¹, 1154 cm⁻¹, 1329 cm⁻¹, 1347 cm⁻¹, 1577 cm⁻¹, 1611 cm⁻¹, 1653 cm⁻¹, 1699 cm⁻¹, 2626 cm⁻¹, 2863 cm⁻¹, 3201 cm⁻¹, 2943 cm⁻¹, 3468 cm⁻¹ and 3565 cm⁻¹.

49. (canceled)

50. (canceled)

51. A pharmaceutical composition which comprises a compound according to claim 40 which is [(2R)-2-(Cyclopentylmethyl)-3-(2-{5-fluoro-6-[(9aS)-hexahydropyrazino[2,1-c][1,4]oxazin-8(1H)-yl]-2-methyl-4-pyrimidinyl}hydrazino)-3-oxopropyl]hydroxyformamide methanesulphonate Form 2 which is a crystalline anhydrate or crystalline anhydrous form.
